(19) United States
(12) Patent Application Publication
(10) Pub. No.: US 2023/0062896 A1
(43) Pub. Date: Mar. 2, 2023

(54) INCREASE OF SATURATED FAT IN SOYBEAN

(71) Applicant: Calyxt, Inc., Roseville, MN (US)

(72) Inventors: Qiwei Shan, Roseville, MN (US); Zachary Demorest, Minneapolis, MN (US); James Presnail, Edina, MN (US)

(73) Assignee: Calyxt, Inc., Roseville, MN (US)

(21) Appl. No.: 17/759,498

(22) PCT Filed: Feb. 1, 2021
(86) PCT No.: PCT/US2021/016091

§ 371 Date: Jul. 26, 2022

Related U.S. Application Data
(60) Provisional application No. 62/968,630, filed on Jan. 31, 2020.

(51) Int. Cl. C12N 15/82; C12N 9/02; C12N 9/16

(52) U.S. Cl. CPC C12N 15/8247; C12N 9/0071; C12Y 114/19002; C12N 9/16; C12Y 301/02014

(57) ABSTRACT

Materials and methods are provided for making soybean varieties that have altered oil composition as a result of one or more mutation modulating the expression of a SACPD-C gene, a FATB-1A gene, or both the SACPD-C and FATB-1A genes. For example, a soybean plant, plant part, or plant cell producing an oil that has increased saturated fatty acid content as compared to oil produced from a corresponding soybean plant, plant part, or plant cell lacking the one or more mutations is provided.

### BACKGROUND

Soybean (Glycine max) is an important legume crop worldwide due to its ability to fix atmospheric nitrogen. Soybeans also serve as a major source of animal feed protein, and its oil has uses ranging from cooking/frying to industrial uses and biodiesel. Soybean oil contains 11% palmitic acid (C16:0), 4% stearic acid (C18:0), 23% oleic acid (C18:1), 54% linoleic acid (C18:2), and 7.6% linolenic acid (C18:3). The total saturated fatty acid (palmitic and stearic) account for about 15% of total fatty acid composition.

The fatty acid composition of soybean oil described above can be less than optimal for use in specific food and cosmetic production applications. While some limitations maybe overcome by chemical hydrogenation, the trans fatty acids produced as a result of partial hydrogenation are associated with unfavorable health effects. Palm oil or palm kernel oil, which both come from the oil palm tree (Elaeis guineensis), have served as a replacement of partially hydrogenated oils in food applications since it is solid at room temperature and its fractions deliver a wide range of functional melting profiles. The oil has a high melting point and is high in saturated fats, which is ideal for creating desirable skin-feel sensations for creams and cosmetics, and appealing mouth feel for confectionaries. Palm oil's unique chemistry can also survive the high temperatures involved in cooking, and the oil's resistance to spoilage confers a long shelf life upon products containing the oil.

The cultivation of palm oil is often connected with issues of environmental sustainability making palm oil an unpopular choice. There is a pressure to find alternatives to palm oil for food and cosmetic uses. One of the major drawbacks of non-palm fats based on exotic fats such as shea, cocoa butter, and others, is the high price of the raw materials. Moreover, these fats may have sustainability concerns as well. Liquid oils like rapeseed oil, sunflower oil or soybean oil cannot replace all palm fractions in every application. It would be highly desirable to be able to provide soybean varieties having sufficiently elevated palmitic acid and stearic acid contents to produce a solid fat having that can be used to duplicate the properties of palm oil fractions.

Improvements to the nutritional and commercial quality of soybean oil could add further value to oil-based products. Alteration of the soybean oil content and composition to increase saturated fatty acid content is needed to provide products of higher nutritional content and greater stability. Moreover, a soybean oil with increased saturated fatty acid content could reduce the need for industrial hydrogenation of polyunsaturated oil for food applications, thereby reducing negative health impacts associated with trans fats.

### SUMMARY

The present disclosure features soybean plants, plant parts, and plant cells producing an oil with elevated saturated fatty acid content. This document provides materials and methods for creating soybean varieties that produce soybeans with a saturated fatty acid content greater than about 15% by weight of the total fatty acid content. The disclosure herein is based at least in part on the discovery that mutations modulating the expression of a SACPD-C gene, a FATB-1A gene, or both the SACPD-C and FATB-1A genes in a soybean plant, plant part, or plant cell can enhance accumulation of saturated fatty acids such as stearic acid and palmitic acid, for the production of a solid fat or fraction thereof having the functional melting profiles of palm oil, cocoa butter or other exotic fats, without hydrogenation. Moreover, the disclosure is based on targeted mutations that modulating the expression of a SACPD-C gene, a FATB-1A gene, or both the SACPD-C and FATB-1A genes that avoid pleiotropic defects associated with random mutagenesis, such as deleterious effects on development seen with full knockout mutants or non-specific overexpression mutants.

Accordingly, one aspect of the present disclosure features a soybean plant, plant part or plant cell comprising one or more mutations modulating the expression of a SACPD-C gene, a FATB-1A gene, or both the SACPD-C and FATB-1A genes, wherein said plant, plant part, or plant cell produces oil that has increased saturated fatty acid content as compared to oil produced from a corresponding soybean plant, plant part, or plant cell lacking the one or more mutations, and wherein the one or more mutations modulating the expression of the SACPD-C gene comprise a targeted mutation induced by a rare-cutting endonuclease. The soybean plant, plant part, or plant cell can include a mutation resulting in reduced expression of the SACPD-C gene. The mutation resulting in reduced expression of the SACPD-C gene can be a mutation in one or more alleles of the SACPD-C gene or an operatively linked promoter thereof. The mutation resulting in reduced expression of the SACPD-C gene can be a knock-out mutation. The knock-out mutation can be a seed-specific knock-out mutation. The seed-specific knock-out mutation can include a replacement of a seed-specific promoter at a native genomic locus of the SACPD-C gene with a promotor with low activity or no detectable activity in developing soybean seed. The mutation resulting in reduced expression of the SACPD-C gene can be in a sequence set forth in SEQ ID NO: 17, 20, 23, 26, 29, 32, 35, 38, 41, 44, or 47. The mutation resulting in reduced expression of the SACPD-C gene can be a knock-in mutation of a functional SACPD-C gene operably-linked to a promotor with low activity or no detectable activity in developing soybean seed. The promoter with low activity or no detectable activity in developing soybean seed in one or more of the embodiments above can be a nodule specific gene promoter. In one or more of the embodiments above, the soybean plant, plant part, or plant cell comprises a mutation resulting in increased expression of the FATB-1A gene. The mutation increasing expression of the FATB-1A gene can be a targeted replacement of the endogenous promoter of the FATB-1A gene with an overexpression promoter. The overexpression promoter can be a strong seed-specific promoter, optionally a FAD2A promoter or a FAD2B promoter.

In another aspect, the present disclosure features a method for generating a soybean plant comprising a mutation modulating the expression of a SACPD-C gene, a FATB-1A gene, or both the SACPD-C and FATB-1A genes, comprising:

(a) contacting a population of soybean plant cells from a soybean plant producing an oil with a saturated fatty acid content of about 15% of total fatty acid composition with one or more nucleic acid sequences inducing:
(i) a mutation resulting in reduced expression of the SACPD-C gene, wherein the mutation is a targeted mutation induced by a rare cutting endonuclease;
(ii) a mutation resulting in increased expression of the FATB-1A gene; or
(iii) a combination thereof;

(b) selecting, from the population, a cell in which expression SACPC-C gene has been reduced, expression of the FATB-1A gene has been increased, or expression SACPC-C gene has been reduced and expression of the FATB-1A gene has been increased, and

(c) regenerating the selected plant cell into a soybean plant.

Reducing expression of the SACPD-C gene can include inducing a mutation in one or more alleles of the SACPD-C gene or an operatively linked promoter thereof. The induced mutation can be a knock-out mutation or a seed-specific knock-out mutation. Reducing expression of the SACPD-C gene can include replacing a seed-specific promoter at a native genomic locus of the SACPD-C gene with a promotor with low activity or no detectable activity in developing soybean seed. Reducing expression of the SACPD-C gene can further include delivering to the population of soybean plant cells an expression cassette comprising a functional SACPD-C gene operably-linked to a promotor with low activity or no detectable activity in developing soybean seed. The promoter with low activity or no detectable activity in developing soybean seed can be a nodule specific promoter. In one or more embodiments above, the method can include increasing expression of the FATB-1A gene by replacing an endogenous promoter of the FATB-1A gene with an overexpression promoter. Increasing expression of the FATB-1A gene can include delivering to the population of soybean plant cells an expression cassette including one or more copies of the FATB-1A gene. The one or more copies of the FATB-1A gene can be operably linked to a strong seed-specific promoter.

In another aspect, the present disclosure features a soybean oil composition, comprising a soybean oil produced by a soybean plant, plant part, or plant cell comprising one or more mutations modulating the expression of a SACPD-C gene, a FATB-1A gene, or both the SACPD-C and FATB-1A genes, wherein the soybean oil has increased saturated fatty acid content as compared to oil produced from a corresponding soybean plant, plant part, or plant cell lacking the one or more mutations and wherein the one or more mutations modulating the expression of the SACPD-C gene comprise a targeted mutation induced by a rare-cutting endonuclease. The soybean oil composition can have a stearic acid content of greater than 10%, a palmitic acid content of greater than 10%, or a saturated fatty acid content of greater than 20%, wherein all percentages are based on the weight of the total fatty acids of the oil.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 shows the expression profile of nodule specific gene Glyma05g01360, Glyma13g44970 and Glyma14g27990 (SACPD-C) in different tissues.

FIG. 2 shows the expression profile of seed specific gene FAD2A (Glyma10g42470), FAD2B (Glyma20g24530) and FATB-1A (Glyma05g08060) in different tissues.

FIGS. 3A-B show Table 1 describing candidate gene expression profile in different tissues after normalization: (A) includes young leaf, flower, one cm pod, pod shell (10 and 14 Days after full bloom (DAF)), root and nodule tissue; and (B) includes seed tissue (10, 14, 21, 25, 28, 35, and 42 DAF).

FIG. 4 shows Table 2 describing TALENs for modulating GmSACPD-C gene expression (SEQ ID NOs: 15-47), according to one or more embodiments of the present disclosure (e.g., Approaches 1A and 1B).

FIG. 5 shows Table 3 describing TALENs for modulating GmFATB-1A gene expression (SEQ ID NOs: 48-62), according to one or more embodiments of the present disclosure (e.g., Approach 1C).

FIGS. 6A-B show representative GmSACPD-C DNA sequences of several confirmed mutant profiles from regenerated T₀ plants. The underlined sequences indicate target sites for TAL effector endonucleases. (A) The wild-type GmSACPD-C sequence is shown in SEQ ID NO:63, and mutant sequences are shown in SEQ ID NOS:64-66. (B) Additional mutant sequences are shown in SEQ ID NOS: 67-70.

FIG. 7 shows an alignment of representative GmSACPD-C DNA sequences of several confirmed mutant profiles from regenerated T₀ plants. The underlined sequences indicate target sites for TAL effector endonucleases. The wild-type GmSACPD-C sequence is shown in SEQ ID NO:71, and mutant sequences are shown in SEQ ID NOS:72-76.

FIG. 8A-B describe Approach 1B for seed specific silencing of SACPD-C. (A) illustrates components of an exemplary geminivirus binary vector for targeting upstream of the SACPD-C coding sequence including a TALEN pair and a donor template. The 2 kb sequence of the nodule-specific promoter and 5′ UTR (Glyma13g44970) is flanked by a Left Homology Arm (LHA) and a Right Homology Arm (RHA). (B) illustrates the gene targeting event upon cleavage by the nuclease and homologous recombination with the donor template replicon. According to one embodiment of the present disclosure, the targeted promoter replacement donor template sequence is set forth in SEQ ID NO: 79 and the expected edited RHA sequence is set forth in SEQ ID NO: 80.

FIG. 9 shows a representative targeted promoter replacement donor template sequence for Approach 1B (SEQ ID NO: 79).

FIG. 10 shows the expected edited RHA sequence using the template of FIG. 9 (SEQ ID NO: 80) to replace the endogenous SACPD-C gene promoter.

FIGS. 11A-B describe Approach 1C for seed specific overexpression (OE) of FATB-1A. (A) illustrates components of an exemplary geminivirus binary vector for targeting upstream of the FATB-1A coding sequence, including a TALEN pair and a donor template, flanked by a LHA and a RHA. (B) illustrates the gene targeting event upon cleavage by the nuclease and homologous recombination with the donor template replicon.

FIG. 12 shows a representative targeted promoter replacement donor template sequence for Approach 1C (SEQ ID NO: 81).

FIG. 13 shows a representation of a DNA construct for Approach 2A to produce a plant, plant part or plant seed with tissue specific expression of SACPD-C and FATB-1A, The construct shown includes two expression cassettes: cassette 1 with the coding sequence of SAPCD-C and the promoter (nodule-specific) and terminator sequences of Glyma13g44970 and cassette 2 with the coding sequence of FATB-A1 and the promoter and terminator sequences of FAD2A. The sequence of a representative construct for Approach 2A is shown in SEQ ID NO: 82.

### DETAILED DESCRIPTION

The present disclosure features soybean plants, plant parts and plant cells that can be used to produce an oil with elevated saturated fatty acid content as a result of one or more mutations that modulate the expression of genes involved fatty acid synthesis within the plant cell, as well methods for generating such plants, and oil derived from such plants. The methods described herein can be used to generate soybean varieties having oil with a stearic acid content of at least 10% and/or a palmitic acid content of at least 10%. In some embodiments, the change in oil composition is achieved by altering the expression of the soybean SACPD-C gene, completely or in a seed-specific manner, or overexpressing the soybean FATB-1A gene. According to some of the methods provided herein, the modifications are achieved using non-transgenic techniques. The targeted mutations described herein minimize or avoid pleiotropic effects that can lead to detrimental phenotypes in soybean crops.

### Definitions

For the present disclosure, terms are defined as follows:

The term “cis-genic” refers to genetic modification of plants with a natural gene, encoding a trait from the plant itself or from a sexually compatible donor plant. Cis-genic modifications are distinguishable from transgenic modification, in which a plant is genetically modified with a gene from a non-crossable species or with a synthetic gene.

An “endogenous gene” refers to a nucleic acid molecule comprising the sequence of the wild-type sequence occurring in the wild-type plant, or a sequence having a percent identity that allows it to retain the function of the encoded product, such as a sequence with at least 90% identity, and may be obtained from the plant or plant part of cell, or may be synthetically produced. Further embodiments provide the sequence has at least 91%, 92%, 93%, 94%, 95%, 96%, 97%, 98%, 99% identity. In embodiments described herein, an endogenous gene nucleotide sequence can be inserted at a different locus than that of the wild-type gene and be operably linked to a different promoter than the wild-type gene.

“SACPD” refers a Δ9-stearoyl-acyl carrier protein desaturase. Accordingly, a “SACPD gene” refers to a gene encoding the Δ9-stearoyl-acyl carrier protein desaturase protein. Three isoforms of the SACPD gene have been identified in soybean. Two of the isoforms of this gene, SACPD-A and SACPD-B, are expressed in both vegetative and reproductive tissues while the third, SACPD-C (Glyma14g27990), is primarily expressed in developing seed and the nodules.

“FATB” refers to a gene encoding palmitoyl-acyl carrier protein thioesterase.

The term “soybean plant” or “plant part” is used broadly to include a soybean plant at any stage of development, or to part of soybean plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered a plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, leaves, stems, seed pods, seeds, roots, nodules, and the like. A part of a plant useful for propagation includes, for example, seeds, seed pods, cuttings, seedlings, rootstocks, and the like. “Seed” refers to any plant structure that is formed by continued differentiation of the ovule of the plant, following its normal maturation point at flower opening, irrespective of whether it is formed in the presence or absence of fertilization and irrespective of whether or not the seed structure is fertile or infertile.

“Expression cassette” means a DNA sequence capable of directing expression of a particular nucleotide sequence in an appropriate host cell, comprising a promoter operably linked to a nucleotide sequence of interest, which is optionally operably linked to termination signals and/or other regulatory elements. An expression cassette may also comprise sequences required for proper translation of the nucleotide sequence. The coding region usually codes for a protein of interest but may also code for a functional RNA of interest, for example antisense RNA or a non-translated RNA, in the sense or antisense direction. The expression cassette comprising the nucleotide sequence of interest may be chimeric, meaning that at least one of its components is heterologous with respect to at least one of its other components. The expression cassette may also be one, which is naturally occurring but has been obtained in a recombinant form useful for heterologous expression. An expression cassette may be assembled entirely extracellularly (e.g., by recombinant cloning techniques). However, an expression cassette may also be assembled using in part endogenous components. For example, an expression cassette may be obtained by placing (or inserting) a promoter sequence upstream of an endogenous sequence, which thereby becomes functionally linked and controlled by said promoter sequences.

### A. SACPD-C Gene Expression Mutants

Embodiments of the present disclosure feature soybean plants, plant parts, or plant cells comprising one or more mutations modulating expression of a SACPD-C gene. The one or more mutations can be present in a coding or non-coding sequence of the SACPD-C gene. The one or more mutations can be present within the SACPD-C gene, i.e., within the open reading frame of the gene, or at a region modulating expression of the SACPD-C gene, i.e., within a regulatory region of a SACPD-C gene, or a combination thereof. For example, a promoter-targeted mutation that disrupts a binding sequence of a SACPD-C promoter can reduce expression of the SACPD-C gene. In some cases, one or more mutations that alter expression of the SACPD-C gene are present in an intronic region, an exonic region, an enhancer region, a promoter region, an untranslated region (UTR 5′ or 3′), or a combination of two or more of these regions of a SACPD-C gene. Genomic sequences of Glycine max SACPD-C genes are publicly available. For example, the native genomic sequence of a SACPD-C gene, Glyma.14g27990, can be downloaded from Soybase Database (www.soybase.org). The mutation modulating expression of a SACPD-C gene can be in one or more alleles of the gene. A representative example of the coding sequence of a naturally occurring Glycine max SACPD-C nucleotide sequence is shown in (SEQ ID NO: 1). The coding CDS does not contain native introns, and encodes the same polypeptide as the native genomic sequence. In some embodiments, soybean plants, cells, plant parts, seeds, and progeny thereof provided herein can have a mutation in each endogenous SACPD-C allele or its promotor (e.g., a seed specific promoter), such that expression of the gene is reduced or completely inhibited in the plant or in a specific tissue. Thus, in some cases, the plants, cells, plant parts, seeds, and progeny do not exhibit detectable levels of Δ9-stearoyl-acyl carrier protein desaturase expressed from the endogenous SACPD-C gene.

In particular embodiments, the expression is reduced by mutations of the SACPD-C gene or its promoter. Reducing the expression of a gene in a plant, plant part or a plant cell includes inhibiting, interrupting, knocking-out, or knocking-down the gene, such that transcription of the gene and/or translation of the encoded polypeptide is reduced as compared to a corresponding control plant, plant cell, or population of plants or plant cells in which expression of the gene or polypeptide is not inhibited, interrupted, knocked-out, or knocked-down. For example, gene knockdown using RNAi technology can be employed. The reduction encompasses any decrease in expression level (e.g., a decrease of 10% or more, 20% or more, 30% or more, 40% or more, 50% or more, 60% or more, 70% or more, 80% or more, 90% or more, or even 100%) as compared to the corresponding control plant, plant cell, or population of plants or plant cells. In some embodiments, reducing expression by 50% or more may be particularly useful. Expression levels can be measured using methods such as, for example, reverse transcription-polymerase chain reaction (RT-PCR), Northern blotting, dot-blot hybridization, in situ hybridization, nuclear run-on and/or nuclear run-off, RNase protection, or immunological and enzymatic methods such as ELISA, radioimmunoassay, and western blotting.

In some cases, the plants, plant cells, plant parts, seeds, and progeny provided herein can be generated using a rare-cutting endonuclease (e.g., a transcription activator-like effector nuclease (TALE-nuclease)) system to make a targeted knockout in one or more alleles of the SACPD-C gene. The gene targeted for knock-out can have a coding sequence as set forth in SEQ ID NO: 1, or a SACPD-C gene with at least 75% sequence identity to SEQ ID NO: 1.

The percent sequence identity between a particular nucleic acid and a sequence referenced by a particular sequence identification number is determined as follows. First, a nucleic acid is compared to the sequence set forth in a particular sequence identification number using the BLAST 2 Sequences (B12seq) program from the stand-alone version of BLASTZ containing BLASTN version 2.0.14. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide residue is presented in both sequences. The percent sequence identity is determined by dividing the number of matches either by the length of the sequence set forth in the identified sequence (e.g., SEQ ID NO: 1), or by an articulated length (e.g., 100 consecutive nucleotides or amino acid residues from a sequence set forth in an identified sequence), followed by multiplying the resulting value by 100. The percent sequence identity value is rounded to the nearest tenth. For example, the knocked out gene can have a coding sequence with at least 80%, at least 85%, at least 90%, at least 91%, at least 92%, at least 93%, at least 94%, at least 95%, at least 96%, at least 97%, at least 98%, or at least 99% sequence identity to SEQ ID NO: 1.

This disclosure provides materials and methods for using rare-cutting endonucleases (e.g., TALE-nucleases) to generate soybean plants and related products (e.g., seeds and plant parts) that are particularly suitable for providing high saturated fatty acid oil, due to targeted knockouts of the SACPD-C gene. Other sequence-specific nucleases also may be used to generate the desired plant material, including engineered meganucleases/homing endonucleases (e.g., I-SceI or I-CreI), zinc finger nucleases (ZFNs), and the clustered regularly interspaced short palindromic repeat (CRISPR)/CRISPR-associated protein 9 (Cas9).

The rare-cutting endonuclease can be a natural or engineered protein having endonuclease activity directed to a nucleic acid sequence with a recognition sequence (target sequence) about 12-40 bp in length (e.g., 14-40, 15-36, or 16-32 by in length; see, e.g., Baker, Nature Methods 9:23-26, 2012). Typical rare-cutting endonucleases cause cleavage inside their recognition site, leaving 4 nucleotide (nt) staggered cuts with 3′OH or 5′OH overhangs. In some embodiments, a rare-cutting endonuclease can be a meganuclease, such as a wild-type or variant homing endonuclease (e.g., a homing endonuclease belonging to the dodecapeptide family (see, WO 2004/067736). Another type of rare-cutting endonuclease is referred to herein as “Cas9/CRISPR system”. This system is characterized by the combined use of an endonuclease from the bacterial Cas9 family and of a single stranded guide RNA that guides said endonuclease to a DNA target sequence generally of 20 base pairs. This DNA target is generally chosen to be located in the genome upstream so-called PAM (protospacer adjacent motif) sequence motives (NGG or NAG) recognized by Cas9. The guide RNA molecule (gRNA), which is generally a single stranded RNA is introduced into the living cell to confer cleavage and specificity to Cas9. It is a synthetic RNA designed to match the desired 20 bp sequence in the genome upstream the PAM. The use of Cas9/CRISPR in plants has been reviewed by Belhaj et al. (2013), which is incorporated by reference. In some embodiments, a rare-cutting endonuclease can be a fusion protein that contains a DNA binding domain and a catalytic domain with cleavage activity. TALE-nucleases and ZFNs are examples of fusions of DNA binding domains with the catalytic domain of the endonuclease FokI. Customized TALE-nucleases are commercially available under the trade name TALEN™ (Cellectis, Paris, France). The specificity of transcription activator-like (TAL) effectors depends on an effector-variable. repeat. Polymorphisms are present primarily at repeat positions 12 and 13, which are referred to herein as the repeat variable-diresidue (RVD).

The RVDs of TAL effectors correspond to the nucleotides in their target sites in a direct, linear fashion, one RVD to one nucleotide, with some degeneracy and no apparent context dependence. This mechanism for protein-DNA recognition enables target site prediction for new target specific TAL effectors, as well as target site selection and engineering of new TAL effectors with binding specificity for the selected sites.

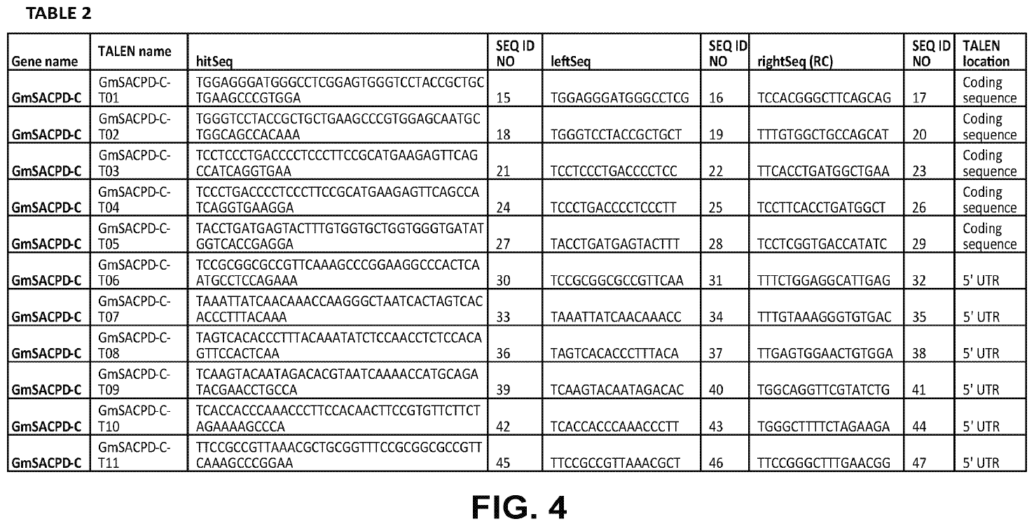

TAL effector DNA binding domains can be fused to other sequences, such as endonuclease sequences, resulting in chimeric endonucleases targeted to specific, selected DNA sequences, and leading to subsequent cutting of the DNA at or near the targeted sequences. Such cuts (double-stranded breaks) in DNA can induce mutations into the wild-type DNA sequence via NHEJ or homologous recombination, for example. In some cases, TALE-nucleases can be used to facilitate site directed mutagenesis in complex genomes, knocking out or otherwise altering gene function with great precision and high efficiency. As described herein, TALE-nucleases targeted to the G. max SACPD-C gene can be used to mutagenize the endogenous gene, resulting in plants or plant tissue without detectable expression of SACPD-C. The fact that some endonucleases (e.g., FokI) function as dimers can be used to enhance the target specificity of the TALE-nuclease. For example, in some cases a pair of TALE-nuclease monomers targeted to different DNA sequences (e.g., the target sequences shown in FIG. 4; SEQ ID NOs: 16 and 17, 19 and 20, 22 and 23, 25 and 26, 28 and 29, 31 and 32, 34 and 35, 37 and 38, 40 and 41, 43 and 44, and 46 and 47) can be used. The relevant sequences useful in the processes include “functional variants” of the sequences disclosed. Functional variants include, for example, sequences having one or more nucleotide substitutions, deletions or insertions and wherein the variant retains desired activity. Functional variants can be created by any of a number of methods available to one skilled in the art, such as by site-directed mutagenesis, induced mutation, identified as allelic variants, cleaving through use of restriction enzymes, or the like.

When the two TALE-nuclease recognition sites are in close proximity, the inactive monomers can come together to create a functional enzyme that cleaves the DNA. By requiring DNA binding to activate the nuclease, a highly site-specific restriction enzyme can be created. Methods for selecting endogenous target sequences and generating TALE-nucleases targeted to such sequences can be performed as described elsewhere. See, for example, U.S. Pat. App. Pub. No. US 2011/0145940 A1 (June 2011), which is incorporated by reference. In some embodiments, software that specifically identifies TALE-nuclease recognition sites.

Methods of the present disclosure includes using rare-cutting endonucleases (e.g., TALE-nucleases) to generate soybean plants, plant cells, or plant parts having mutations in one or more endogenous genes. For example, one or more nucleic acids encoding TALE-nucleases targeted to selected SACPD-C sequences (e.g., the SACPD-C sequences, “hitSeq” shown in TABLE 2 (FIG. 4, i.e., SEQ ID NOs: 15, 18, 21, 24, 27, 30, 33, 36, 39, 42, or 45, or a sequence having at least 95% identity to a sequence in Table 2) can be transformed into plant cells (e.g., protoplasts), where they can be expressed. In some cases, one or more TALE-nuclease proteins can be introduced into plant cells (e.g., protoplasts). The cells, or a plant cell line or plant part generated from the cells, can subsequently be analyzed to determine whether mutations have been introduced at the target site(s), through nucleic acid-based assays or protein-based assays to detect expression levels as described above, for example, or using nucleic acid-based assays (e.g., PCR and DNA sequencing, or PCR followed by a T7E1 assay) to detect mutations at the genomic loci. In a T7E1 assay, genomic DNA can be isolated from pooled calli, and sequences flanking TALE-nuclease recognition sites for SACPD-C can be PCR-amplified. Amplification products then can be denatured and re-annealed. If the re-annealed fragments form a heteroduplex, T7 endonuclease I cuts at the site of mismatch. The digested products can be visualized by gel electrophoresis to quantify mutagenesis activity of the TALE-nuclease. In some embodiments, a method as provided herein can include contacting a population of soybean plant cells (e.g., protoplasts) having a functional SACPD-C allele with a rare-cutting endonuclease that is targeted to an endogenous SACPD-C sequence, selecting from the population a cell in which at least one SACPD-C alleles have been inactivated, and growing the selected cell into a soybean plant. The plant may produce an oil having elevated saturated fatty acid levels, as compared to a control soybean plant that does not contain the inactivated SACPD-C alleles. The rare-cutting endonuclease can be introduced into the population of cells via a nucleic acid (e.g., a vector or a mRNA) that encodes the rare-cutting endonuclease, or as a protein. In some cases, a method as provided herein can include a step of culturing a plant cell containing the inactivated SACPD-C allele(s) to generate one or more plant lines. In addition, or alternatively, a method as provided herein can include a step of isolating genomic DNA containing at least a portion of the SACPD-C locus from the plant cells.

In some embodiments, methods for delivering sequence-specific nucleases to a soybean plant can include Agrobacterium-mediated transformation of plant parts or plant cells (e.g., leaves, stems, petiole, internode explants, callus, or protoplasts) with T-DNA encoding the sequence-specific nucleases, biolistic transformation of plant parts or plant cells with one or more nucleic acids encoding the sequence-specific nucleases, and/or cell-penetrating peptide-mediated transformation of plant parts or plant cells with purified sequence-specific nucleases or nucleic acids (RNA or DNA) encoding the sequence-specific nucleases. Biolistic transformation utilizes particle bombardment. This method has been employed to deliver genome-editing reagents in various crop plants, including soybean. Particle bombardment is based on the direct delivery of nucleic acid sequences into plant cells using metal particles (e.g., gold or tungsten particles). The system can be adapted to deliver protein. The nucleic acid sequences to be delivered can be DNA, including large DNA fragments and RNA, such as mRNA. Using DNA as an example, the method includes coating the particles with the DNA, and shooting the coated particles at plant tissue at high velocity, for the purpose of penetrating plant tissues and cell walls, whereby some particles become lodged inside plant cells. Once inside the cell, the DNA elutes off the particles and becomes transiently expressed or stably integrates into the host genome. The physical delivery of the nucleic acid sequences to cells circumvents host-range limitations sometimes encountered with Agrobacterium and without the necessity of a binary vector. Various tissues and cell types can be transformed by particle bombardment. Multiple plasmids can be delivered with high frequencies of co-transformation. Further methods of delivery include insect vectors, grafting, or DNA abrasion, according to methods that are standard in the art.

In some embodiments, soybean lines having mutations in one or more SACPD-C alleles can be generated by polyethylene glycol (PEG) mediated transformation. For example, protoplasts can be isolated from surface sterilized leaves, and transformed in the presence of PEG with plasmids encoding one or more sequence specific nucleases. Transformation efficiencies can be monitored by delivery of a detectable marker such as a YFP plasmid, which can be visualized using fluorescence microscopy or flow cytometry. After PEG-mediated transformation, protoplasts can be cultured using methods and media known to the person of ordinary skill in the art of protoplast culturing. After a suitable length of time in culture, protoplast-derived calli identified as mutants can be grown, transferred to shoot-inducing medium, and then (once roots form) transferred to soil and grown to maturity for seed production.

In some embodiments, delivery of one or more sequence-specific nucleases to a soybean plant can be achieved through transient delivery or stable integration into the host genome. To transiently deliver sequence-specific nucleases, transformed soybean plant parts or plant cells (using the above-described methods) can be placed on regeneration medium containing no selective agent, and soybean plants can be regenerated. Regenerated plants then can be screened to identify those containing nuclease-induced mutations. To stably integrate the genome engineering reagents into the host genome, nucleic acids encoding the sequence-specific nucleases can be co-delivered with nucleic acid encoding a plant selectable marker. The selectable marker can be harbored on the same vector as the sequence-specific nuclease(s), or can be delivered as a separate vector. After transformation, soybean plant parts or plant cells can be placed on regeneration medium containing the appropriate selectable agent, and transgenic soybean plants can be regenerated. In preferred embodiments, the soybean plants do not include a transgene.

In some embodiments, a nuclease can be co-delivered to a plant cell, using a delivery method described herein (e.g., particle bombardment), with a plasmid encoding one or more exonuclease proteins to increase sequence specific nuclease induced mutagenesis efficiency. Such exonucleases include, without limitation, members of the TREX (therapeutic red cell exchange exonucleases) family of exonucleases, such as TREX2. Other exonucleases also can be used in the methods provided herein.

Another genome engineering tool that can be used in the methods provided herein is based on the RNA-guided Cas9 nuclease from the type II prokaryotic CRISPR (Clustered Regularly Interspaced Short Palindromic Repeats) adaptive immune system. This system allows for cleavage of DNA sequences that are flanked by a short sequence motif, referred as proto-spacer adjacent motif (PAM). Cleavage is achieved by engineering a specific crRNA that is complementary to the target sequence. The crRNA associates into a living cell with a heterologously expressed Cas9 endonuclease. In the crRNA/Cas9 complex, a dual tracrRNA:crRNA structure acts as a guide RNA that directs the Cas9 endonuclease to the cognate target sequence. PAM motifs present in a soybean SACPD-C gene permit design of crRNA specific to SACPD-C gene to introduce mutations or to inactivate one or more SACPD-C alleles within soybean plant cells into which the Cas9 endonuclease and the crRNA are transfected and then expressed. In some embodiments, therefore, this approach can be used to obtain SACPD-C mutant plants as described herein.

The expression of plant genes can be altered by inserting a copy of the nucleic acid sequence which comprises the genomic or coding sequence of plant genes into different genomic loci from the loci of the gene in the plant. The copy of the genomic or coding sequence is operably linked to a promotor and wherein the different genomic loci have transcriptional activity. The sequence to be inserted can be cis-genic or endogenous, and can be obtained from a plant or synthetically created. In some cases, the methods provided herein can involve the targeted knockout of the original endogenous SACPD-C gene and the insertion of a SACPD-C expression cassette comprising the coding sequence of a cis-genic SACPD-C gene operably linked to a promoter providing the desired expression profile at another genomic locus. By “operably linked”, the respective coding sequence is fused in-frame to the promoter, so that the coding sequence is faithfully transcribed, spliced, and translated. The genomic locus where the cassette is inserted can be a location within the genome different than the original endogenous SACPD-C gene. The locus can be on a different chromosome than the original endogenous SACPD-C gene, or the same chromosome. Preferably, where the insertion is on the same chromosome as the endogenous gene, the insertion's genomic locus will not capture the transcriptional activity of the promoter from the original endogenous SACPD-C gene.

The promoter is an expression control sequence composed of a region of a DNA molecule, typically upstream of the point at which transcription starts (generally near the initiation site for RNA polymerase II). Promoters are involved in recognition and binding of RNA polymerase and other proteins to initiate and modulate transcription. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element. The choice of promoters useful in the methods depends upon the type of desired expression to be achieved. Factors, including, but not limited to, efficiency, selectability, inducibility, desired expression level, and cell or tissue specificity. For example, tissue-, organ- and cell-preferred promoters that confer transcription only or predominantly in a particular tissue, organ, and cell type, respectively, can be used. In some embodiments, promoters specific to vegetative tissues such as the stem, parenchyma, ground meristem, vascular bundle, cambium, phloem, cortex, shoot apical meristem, lateral shoot meristem, root apical meristem, lateral root meristem, leaf primordium, leaf mesophyll, or leaf epidermis can be suitable regulatory regions. In some embodiments, promoters that are not active in seeds can be useful. Other classes of promoters include, but are not limited to, inducible promoters, such as promoters that confer transcription in response to inducers, such as external stimuli such as chemical agents, developmental stimuli, or environmental stimuli. The promoter may be one which preferential expresses to particular tissue, organ or other part of a plant, or may express during a certain stage of development or under certain conditions. When referring to preferential expression, what is meant is expression at a higher level in the particular plant tissue than in other plant tissue.

A promoter of interest may have strong or weak transcriptional activity. A skilled person appreciates a promoter sequence can be modified to provide for a range of expression levels of and operably linked heterologous nucleic acid molecule. Generally, by “weak promoter” is intended a promoter that drives expression of a coding sequence at a low level. By “low level” is intended levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts. It is recognized that to increase transcription levels, enhancers can be utilized in combination with the promoter regions.

Altering expression with a knock-out mutation of the original endogenous SACPD-C gene can result in all SACPD-C gene expression being controlled from the operably linked promoter. For the knocked-in SACPD-C, a nodule specific promoter may be desired. In some cases, the method comprises identifying an endogenous gene that matches the desired expression profile, and cloning the regulatory elements of the endogenous gene. For example, suitable promoters for inclusion in an expression cassette for nodule-specific SACPD-C expression can include, for example, the promoter of nodule specific genes Glyma05g01360 and Glyma13g44970. SEQ IDs 2-5 shows the promoter and terminator sequences for Glyma05g01360 and Glyma13g44970. In other situations, tissue specific, stage specific or inducible expression may be desired.

Any method which provides for efficient transformation may be employed. For example, methods for plant cell transformation include the use of Ti- or Ri-plasmids, microinjection, electroporation, DNA particle bombardment, liposome fusion, or the like. In many instances, it will be desirable to have the construct bordered on one or both sides by T-DNA, particularly having the left and right borders, more particularly the right border. This is particularly useful when the construct uses A. tumefaciens or A. rhizogenes as a mode for transformation, although the T-DNA borders may find use with other modes of transformation.

In one embodiment, the methods provided herein can involve the targeted knockout of the original endogenous SACPD-C gene and the targeted insertion of SACPD-C (genomic or CDS) into a locus with a gene sequence near a promoter of interest. Knocking out the original endogenous SACPD-C gene can result in all SACPD-C gene expression being controlled from the promoter of interest. Preferably, the promoter is not active in developing seeds, for example, a nodule- or root-specific promoter. In other situations, stage specific or inducible expression may be desired. In some cases, the method comprises identifying an endogenous gene that matches the desired expression profile. Several methods and software programs are available for identifying genes with desired expression characteristics. These include, but are not limited to RNA-sequencing (whole transcriptome shotgun sequencing). Once genes with desired expression profiles are identified, it is to be understood that the promoter sequence (usually upstream or nearby the gene of interest) is a key component used in the method, as opposed to the actual gene being expressed by the promoter. The last step is to determine the specific type of genome edit that is required to capture the transcriptional activity of the identified promoter.

In some cases, the methods provided herein can involve the seed-specific knockout of the SACPD-C gene. For example, geminivirus sequences can be used as gene targeting vectors to target and replace endogenous promotors of SACPD-C gene with a promoter that is not active in developing seeds. Geminiviruses are a large family of plant viruses that contain circular, single-stranded DNA genomes, the sequences of which can be used as gene targeting vectors. For example, the geminivirus genome can be engineered to contain a desired modification flanked by sequences of homology to a target locus. In some cases, this can be accomplished by replacing non-essential geminivirus nucleotide sequence (e.g., CP sequence) with a desired repair template. Examples of geminiviruses include the cabbage leaf curl virus, tomato golden mosaic virus, bean yellow dwarf virus, African cassava mosaic virus, wheat dwarf virus, miscanthus streak mastrevirus, tobacco yellow dwarf virus, tomato yellow leaf curl virus, bean golden mosaic virus, beet curly top virus, maize streak virus, and tomato pseudo-curly top virus.

Accordingly, the repair template contains homology to the promotor sequence of the endogenous SACPD-C gene. Typically, a repair template includes a nucleic acid that will replace an endogenous target sequence within the plant, flanked by sequences homologous to endogenous sequences on either side of the target. The flanking homologous sequences can be referred to as “homologous arms”. In this case, the endogenous sequence is replaced with one of the promoter sequences described above. Within the repair template, the flanking homologous sequences can have any suitable length. A suitable length for the flanking homologous sequences will be related to the length of the desired replacement. Therefore, the length can be at least about 25 nt and include sequences that are 750 nt, or longer. In some cases, the flanking homologous sequences can be longer than 800 nt, 900 nt, or longer than 1,000 nt. Repair templates and DNA virus plasmids can be prepared using techniques that are standard in the art. The construct(s) containing the repair template can be delivered to a plant cell using, for example, biolistic bombardment. Alternatively, the repair template can be delivered using Agrobacterium-mediated transformation, insect vectors, grafting, or DNA abrasion, according to methods that are standard in the art.

In addition to the repair template, this method involves an endonuclease that can be customized to target a particular nucleotide sequence and generate a double strand break at or near that sequence. Examples of such customizable endonucleases include ZFNs, Meganucleases, and TALE nucleases, as well as CRISPR/Cas systems described above. Like TALE nucleases, for example, the components of a CRISPR/Cas system (the Cas9 endonuclease and the crRNA and tracrRNA, or the cr/tracrRNA hybrid) can be delivered to a cell in a geminivirus construct.

After a plant is infected or transfected with a repair template and the associated endonuclease, any suitable method can be used to determine whether the seed-specific knockout of the endogenous SACPD-C gene occurred. For example, PCR-based methods also can be used to ascertain whether a genomic target site contains the repair template sequence, and/or whether precise recombination has occurred at the 5′ and 3′ ends of the repair template.

The disclosed strategies can combine conventional breeding with the targeted approaches described above. The SACPD-C gene expression targeted gene edits can be implemented on any strain, species or cultivar of soybean that is of interest, without limitation. In some embodiments, the targeted gene edits or other genetic modifications can be implemented in germplasm or other plant tissue that already possesses characteristics (e.g., genetic predisposition) for producing an oil with an elevated saturated oil content.

### B. FATB-1A Gene Expression Mutants

Embodiments of the present disclosure feature soybean plants, plant parts, or plant cells comprising one or more mutations modulating expression of a FATB-1A gene. The one or more mutations can be in a regulatory region of a FATB-1A gene, such as an enhancer region, a promoter region, a UTR region (5′ or 3′), a silencer region, or a combination of regions of a FATB-1A gene. Genomic sequences associated with the Glycine max FATB-1A locus are publicly available. For example, the sequence of the native soybean FATB-1A gene, Glyma05g08060, can be downloaded from Soybase Database (www.soybase.org). The mutation can be at a different genomic locus than the endogenous FATB-1A gene. For example, the coding sequence of a naturally occurring G. max FATB-1A nucleotide sequence (e.g., a representative sequence is shown in (SEQ ID NO: 6)) can be inserted into any locus of the genome, or into a plurality of loci, thereby providing at least two functional FATB-1A genes. The coding CDS does not contain native introns, and encodes the same polypeptide as the native genomic sequence such that expression of the gene is elevated or increased in the plant or in a specific tissue (e.g., in developing seeds). Thus, in some cases, the plants, cells, plant parts, seeds, and progeny exhibit elevated levels of acyl-ACP thioesterase expressed from one or more soybean FATB-1A genes.

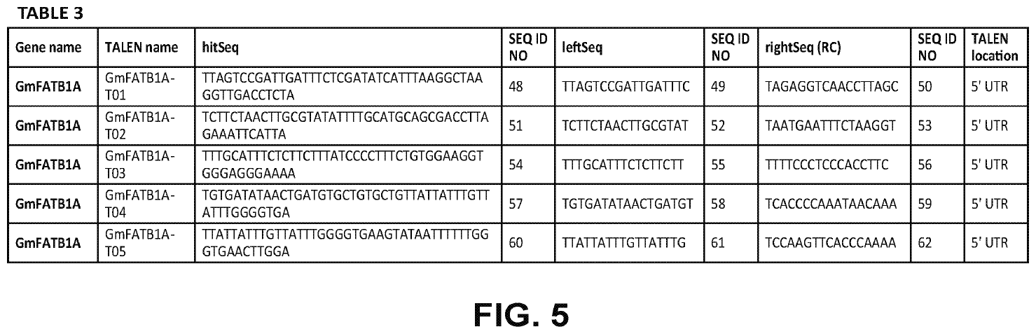

The gene editing techniques described above for modulating expression of the SACPD-C gene can be modified to enhance expression of FATB-1A. In one or more embodiments, the methods provided herein can involve the targeted replacement of the FATB-1A promoter with an overexpression promoter. The promoter can be a native soybean promoter, which can be seed-specific promoter such as a promoter of genes encoding (3-conglycinin and lectin. Suitable promoters can be selected based on expression profile of seed specific genes. In some cases, the method comprises identifying an endogenous gene that matches the desired expression profile. Several methods and software programs are available for identifying genes with desired expression characteristics. These include, but are not limited to RNA-sequencing (whole transcriptome shotgun sequencing). Once genes with desired expression profiles are identified, it is to be understood that the promoter sequence (usually upstream or nearby the gene of interest) is a key component used in the method, as opposed to the actual gene being expressed by the promoter. The last step is to determine the specific type of genome edit that is required to capture the transcriptional activity of the identified promoter. A suitable seed-specific promoter can be one that drives expression at a specific stage of development. Preferably, the promoter will provide high expression in developing seeds. More preferably, the high expression in seeds, e.g., developing seeds, is combined with no or very low level expression in other tissue. In some cases, the promoter is from a gene encoding a fatty acid desaturase enzyme. For example, as described in the Examples, the expression profiles of GmFAD2A (Glyma10g42470) and GmFAD2B (Glyma20g24530) have been identified by the inventors as suitable candidates for driving the overexpression of FATB-1A. Therefore, a geminivirus can be designed to target and replace the endogenous FATB-1A promoter with the promoter of the endogenous FAD2A or FAD2B gene. TALENs targeting the FATB-1A 5′-UTR region can be designed based on the 5′-UTR sequence. Exemplary TALENs are presented in FIG. 5 (Table 3) showing SEQ ID NOs: 49 and 50, 52 and 53, 55 and 56, 58 and 59, and 61 and 62.

In some cases, the mutation enhancing expression of soybean FATB-1A can be untargeted. For example, an expression cassette comprising a coding sequence of a soybean FATB-1A gene operably linked to a strong promoter or a seed promoter can be inserted into any genomic locus (e.g., by biolistic methods). A suitable cis-genic promotor can be selected based on the desired expression profile. For example, promoters can be selected based on high expression in developing seeds, and no or low levels of expression in other tissues. In some cases, the operably linked promoter can be a sequence as set forth in SEQ ID NOs: 7 or 8. The cassette can include the termination sequences of GmFAD2A (Glyma10g42470) or GmFAD2B (Glyma20g24530) (SEQ ID NOs: 9 and 10, respectively).

Increased expression encompasses any degree of increase in the total expression level (e.g., an increase of 10% or more, 20% or more, 30% or more, 40% or more, 50% or more, 60% or more, 70% or more, 80% or more, 90% or more, or even 100% or more) as compared to the corresponding control plant, plant cell, or population of plants or plant cells. For example, expression can be increased by about 2-fold, about 5-fold, or about 10-fold as compared to the control plant, plant cell, or population thereof. Expression levels can be measured using methods such as, for example, reverse transcription-polymerase chain reaction (RT-PCR), Northern blotting, dot-blot hybridization, in situ hybridization, nuclear run-on and/or nuclear run-off, RNase protection, or immunological and enzymatic methods such as ELISA, radioimmunoassay, and western blotting.

The disclosed strategies can combine conventional breeding with the targeted approaches described above. The FATB-1A gene expression targeted gene edits can be implemented on any strain, species or cultivar of soybean that is of interest, without limitation. In some embodiments, the targeted gene edits or other genetic modifications can be implemented in germplasm or other plant tissue that already possesses characteristics (e.g., genetic predisposition) for producing an oil with an elevated saturated oil content.

### C. Stacked Traits

Embodiments featuring soybean plants, plant parts or plant cells having mutations that modulate the expression of both the SACPD-C and FATB-1A genes, wherein said plant, plant part, or plant cell produces oil that has increased saturated fatty acid content as compared to oil produced from a corresponding soybean plant, plant part, or plant cell lacking the one or more mutations are within the scope of this disclosure. In particular, the present disclosure features plant lines with more than one mutation directed to increasing the saturated fatty acid content of oil produced. In some cases, a plant line can provide transcription or transcription and translation of one or more other sequences of interest in concert with the tissue specific expression of the soybean stearoyl-ACP desaturase and/or overexpression of the soybean stearoyl-ACP thioesterase.

Providing a plant transformed for a combined effect can involve the use of multiple separate nucleic acid constructs or transformation events. For example, multiple constructs as described above may be introduced into a plant cells by the same or different methods, including the introduction of such a trait by the inclusion of two transcription cassettes in a single transformation vector, the simultaneous transformation of two expression constructs, retransformation using plant tissue expressing one construct with an expression construct for the second gene, or by crossing transgenic plants via traditional plant breeding methods, so long as the resulting product is a plant having both characteristics integrated into its genome. In some cases, a soybean plant is transformed using a construct described above, regenerated. Regenerated plants that possess the desired sequences are selfed to remove the gene editing plasmid and retain the targeted mutation. Lines of resulting null segregant plants with specific mutations can then be crossed to provide a plant seed or plant exhibiting a combined effect. For example, null segregants of a seed specific SACPD-C knock-out line can be crossed with null segregants of a seed specific FATB-1A overexpression line, and null segregants of a ubiquitous knock-out SACPD-C line can be crossed with null segregants that overexpress both SACPD-C and FATB-1A in a seed-specific manner.

Any combination of approaches can be utilized to achieve combined GmSACPD-C and GmFATB-1A gene modulation. For example, combined modulation can include TALEN-mediated knock-out of one or more SACPD-C alleles, insertion of a first linear cis-genic cassette comprising a Nodule promoter operably linked to the coding sequence of GmSACPD-C, and insertion of a second linear cis-genic cassette comprising a Seed promoter operably linked to the coding sequence of GmFATB-1A. In some cases, the first linear cis-genic cassette has the sequence set forth in SEQ ID NO: 13, which includes Nodule Glyma13g44970 promoter-GmSACPD-C-Glyma13g44970 terminator, and the second cis-genic cassette has the sequence set forth in SEQ ID NO: 14, which includes Seed FAD2A promoter-GmFATB1A-FAD2A terminator.

One or more soybean plants can be obtained from individual, mutagenized plant cells (and plants grown therefrom), and at least one of the plants can be identified as containing a mutation modulating expression of a SACPD-C gene or FATB-1A gene. A population of soybean plants sharing a common gene pool and be provided. For example, “M₀” can be used to refer to plant cells (and plants grown therefrom) exposed to a TAL effector nuclease, while “M₁” refers to seeds produced by self-pollinated M₀ plants, and plants grown from such seeds. “M₂ is the progeny (seeds and plants) of self-pollinated M₁ plants, “M₃” is the progeny of self-pollinated M₂ plants, and “M₄”, “M₅”, “M₆” etc. are each the progeny of self-pollinated plants of the previous generation. The term “selfed” as used herein means self-pollinated.

In some cases, at least one of the plants can be identified as containing a mutation in the SACPD-C gene and at least one of the plants can be identified as containing a knocked-in SACPD-C gene. A soybean plant carrying mutant alleles can be used in a plant breeding program to create novel and useful lines and varieties. Thus, in some embodiments, soybean plant containing a mutation in the endogenous SACPD-C gene is crossed with a second soybean plant containing at least one insertion of SACPD-C gene operably linked to a promoter that does not drive expression in developing seeds, and progeny of the cross are identified in which the gene mutations are present. In other embodiments, soybean plant containing at least one mutation modulating expression of SACPD-C gene and at least one mutation modulating expression of a FATB-1A gene is crossed with a second soybean plant, and progeny of the cross are identified in which the gene mutations are present. It will be appreciated that the second soybean plant can contain the same mutations as the plant to which it is crossed, different mutations, or be wild-type with respect to SACPD-C or FATB-1A gene expression.

Breeding can be carried out via known procedures. DNA fingerprinting, SNP or similar technologies may be used in a marker-assisted selection (MAS) breeding program to transfer or breed mutations modulating expression of SACPD-C or FATB-1A alleles into other soybean plants. For example, a breeder can create segregating populations from hybridizations of a genotype containing a mutant allele with an agronomically desirable genotype. Plants in the F₂ or backcross generations can be screened using markers developed from mutant sequences or fragments thereof. Plants identified as possessing the mutation can be backcrossed or self-pollinated to create a second population to be screened. Depending on the expected inheritance pattern or the MAS technology used, it may be necessary to self-pollinate the selected plants before each cycle of backcrossing to aid identification of the desired individual plants. Backcrossing or other breeding procedure can be repeated until the desired phenotype of the recurrent parent is recovered.

Successful crosses yield F₁ plants that are fertile and that can be backcrossed with one of the parents if desired. In some embodiments, a plant population in the F₂ generation is screened for SACPD-C and FATB-1A gene expression, e.g., a plant is identified that fails to express SACPD-C in the developing seed and overexpresses FATB-1A due to the mutations according to standard methods. Selected plants are then crossed with one of the parents and the first backcross (BC₁) generation plants are self-pollinated to produce a BC₁F₂ population that is again screened for variant gene expression. The process of backcrossing, self-pollination, and screening is repeated, for example, at least four times until the final screening produces a plant that is fertile and reasonably similar to the recurrent parent. This plant, if desired, can be self-pollinated, and the progeny subsequently can be screened again to confirm that the plant lacks SACPD-C expression in the developing seed and overexpresses FATB-1A. Cytogenetic analyses of the selected plants optionally can be performed to confirm the chromosome complement and chromosome pairing relationships. Breeder's seed of the selected plant can be produced using standard methods including, for example, analyses of oil to determine the level of saturated fatty acids, including stearic acid and palmitic acid.

In situations where the original F₁ hybrid resulting from the cross between a first, mutant soybean parent and a second, wild-type soybean parent, is hybridized or backcrossed to the mutant soybean parent, the progeny of the backcross can be self-pollinated to create a BC₁F₂ generation that is screened for the mutations.

The result of a plant breeding program using the mutant soybean plants described herein can be novel and useful lines and varieties. As used herein, the term “variety” refers to a population of plants that share constant characteristics which separate them from other plants of the same species. A variety is often, although not always, sold commercially. While possessing one or more distinctive traits, a variety can be further characterized by a very small overall variation between individuals within that variety. A “pure line” variety may be created by several generations of self-pollination and selection, or vegetative propagation from a single parent using tissue or cell culture techniques. A variety can be essentially derived from another line or variety. A variety is “essentially derived” from an initial variety if: a) it is predominantly derived from the initial variety, or from a variety that is predominantly derived from the initial variety, while retaining the expression of the essential characteristics that result from the genotype or combination of genotypes of the initial variety; b) it is clearly distinguishable from the initial variety; and c) except for the differences which result from the act of derivation, it conforms to the initial variety in the expression of the essential characteristics that result from the genotype or combination of genotypes of the initial variety. Essentially derived varieties can be obtained, for example, by the selection of a natural or induced mutant, a somaclonal variant, a variant individual from plants of the initial variety, backcrossing, or transformation. A “line” as distinguished from a variety most often denotes a group of plants used non-commercially, for example in plant research. A line typically displays little overall variation between individuals for one or more traits of interest, although there may be some variation between individuals for other traits.

The methods provided herein can be used to produce plant parts (e.g., seeds) or plant products (e.g., oil) having increased saturated fatty acid content, as compared corresponding plant parts or products from wild-type plants. The fatty acid content of a plant part or a plant product can be evaluated using standard methods.

### D. High Saturated Fatty Acid Soybeans and Uses Thereof

The mutations described herein provide a soybean plant, plant part, or plant cell that can produce an oil with increased saturated fatty acid content as compared to oil produced from a corresponding soybean plant, plant part, or plant cell lacking the one or more mutations.

In one or more embodiments of the present disclosure, the mutations result in a soybean plant, plant part, or plant cell that can produce an oil comprising a total saturated fatty acid content of at least about 20%, at least about 25%, at least about 30%, at least about 35%, at least about 40%, at least about 45% and up to about 80%. The total saturated fatty acid content is preferably within the range of about 20% to about 50%. The percentages of the fatty acids set forth herein, unless otherwise designated, thus are on a weight basis. Oil extracted from the soybean seeds produced by the soybean plant will possess increased stability and superior cooking characteristics compared with an oil extracted from standard soybean seeds, having lower saturated fatty acid content. In addition, the oil has higher levels of solids than commodity soybean oil, making it a more preferred material for the preparation of food products such as margarine, soy flour, soymilk, and shortening. Interesterification of the oil can further enhance the solids content, and the oil's utility in the preparation of food products. The higher saturated fatty acid content can provide a replacement for palm oil fractions or cocoa butter, for example.

The higher saturated fatty acid content can be the result of one or more of increased levels of stearic acid and increased levels of palmitic acid. For example, embodiments of the present disclosure include soybean oil having a stearic acid content of at least about 10%, such as about 10-24% of the total fatty acid composition, soybean oil having a palmitic acid content of at least about 10%, such as about 10-24% of the total fatty acid composition, and combinations thereof. For example, the palmitic acid concentration obtained can be at least about 14% of the total fatty acid composition whereas the stearic acid concentration obtained is at least about 10% of the total fatty acid composition, or the palmitic acid content of the soybean lines of the present invention can be at least about 10%, whereas the stearic acid content is at least about 20% or more. The particular application will generally dictate the desired total saturated fatty acid content. For example, the relative levels of the palmitic and stearic acid contents can be varied to tailor the specific acid contents to the particular needs of the application.

Embodiments of the present disclosure include soybeans having the desired palmitic and stearic acid content, and with various contents of oleic, linoleic, and linolenic acids. The levels of these fatty acids and others can be adjusted for particular applications. For example, it is within the scope of the present invention to alter the soybean plants, plant parts, and plant cells described herein by inclusion (by genetic alteration or by other means) of other fatty acids as may be desired.

The soybeans and their extracted oils may be used in a variety of applications. For example, the soybean oil described herein can be used to replace palm oil, cocoa butter, or other exotic oil, in part or entirely. The soybean oil can serve as a feedstock for cost-effective blending with other saturates and/or interesterification of triacylglycerol. Food applications include, for example, margarine and shortenings, and products containing these ingredients (e.g., baked goods and confectionary). The high saturated fatty acid content is also advantageous for skin care compositions. For example, palmitic acid promotes natural oil regeneration, aiding the skin in retaining its protective barrier. Stearic acid acts primarily as a lubricant. It allows the skin to retain the proper moisture balance. Accordingly, the extracted soybean oil can be included in topical compositions including creams, lotions, and spray oils, that can be applied easily on the hair, skin and nails.

The high saturated fatty acid soybeans can be used in the production of soybean-based food products, such as tofu and soymilk. In addition, the soybeans can be ground into full fat soy flour, which can be used in candies, gravies, sauces, frozen desserts, pastas, meat products, and baked goods. The soy flour can be used to increase the protein content of baked products without affecting the texture.

### Examples

The following Examples are intended to illustrate the above invention and should not be construed as to narrow its scope. One skilled in the art will readily recognize that the Examiners suggest many other ways in which the invention could be practiced. Numerous variations and modifications may be made while remaining within the scope of the invention.

I. Expression Profile of SACPD Genes

Most sources of identified variation for stearic acid content in soybean oil involve mutations in the Δ9-stearoyl-acyl carrier protein desaturase (SACPD) genes. These enzymes desaturate stearoyl-ACP to oleoyl-ACP in the plastid. Three isoforms of SACPD have been identified in soybean. Two of the isoforms, SACPD-A and SACPD-B, are active in both vegetative and reproductive tissues while the third, SACPD-C (Glyma14g27990), is primarily expressed in developing seed and the nodules. In the soybean chemically mutagenized SACPD-C mutant lines are reported as producing oil with a stearate content ranging from 6-14%, which is 1.5 to 3 times the levels contained in wild-type seed of the Williams 82 cultivar. Candidate gene sequencing revealed that all of these lines carried amino acid substitutions in the gene encoding SACPD-C required for the conversion of stearic acid to oleic acid. Further studies also revealed that SACPD-C mutants has some defects in nitrogen fixing nodules and leaf structure.

II. Expression Profile of FatA and FatB Genes

As the terminal step in the fatty acid biosynthesis pathway, acyl-acyl carrier protein (ACP) thioesterases determine the chain length of acyl groups leaving the plastid for further metabolism in the cytosol. Higher plant acyl-ACP thioesterases can be divided into two distinct classes based on amino acid sequence. Referred to as FatA and FatB, FatA thioesterases primarily hydrolyze unsaturated 16:1 and 18:1-ACP, FatB for C8-C16 saturated acyl-ACPs, respectively.

III. Modulating Expression of GmSACPD

To completely inactivate or knock-out the GmSACPD-C gene (A.K.A “ubiquitous KO”), sequence-specific nucleases were designed. TAL effector endonuclease pairs were designed to target GmSACPD-C on the first exon (FIG. 4 showing Table 2).

TAL effector endonucleases are chosen for expression in soybean cells. The activities of these TAL effector endonucleases were assessed at their endogenous target sites in soybean. Each TAL effector endonuclease is cloned into a T-DNA vector downstream of an inducible promoter and then transformed into a strain of Agrobacterium rhizogenes, which are then used to infect half-cotyledons of soybean and produce transgenic hairy roots. Three weeks after infection, hairy roots are collected and frozen in liquid nitrogen, and genomic DNA was prepared using standard methods.

To determine if NHEJ-mediated mutations are created by the TAL effector endonucleases at the target sites in the soybean genome, DNA from nine hairy roots are subjected to a PCR enrichment assay. Samples with TAL effector endonuclease-induced NHEJ mutations may lack the restriction enzyme site within the spacer sequence, resulting in an undigested PCR product which appears as a full-length band on the gel. Thus, undigested PCR products are observed for the GmSACPD-C gene.

Undigested PCR products are cloned and sequenced to verify that they contain TAL effector endonuclease-induced mutations. The PCR products are cloned using a commercially available cloning kit according to manufacturer's instructions. Individual clones derived from a given undigested fragment are sequenced, and the DNA sequences aligned with the wild-type GmSACPD-C gene sequences.

Plants comprising an inactivated or knocked-out GmSACPD-C gene are grown to assess nodulation phenotype.

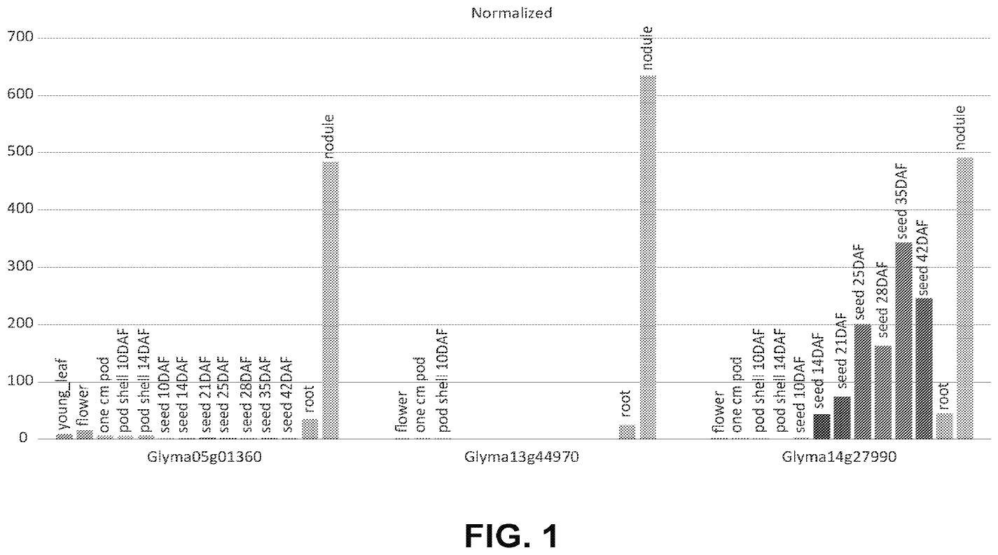

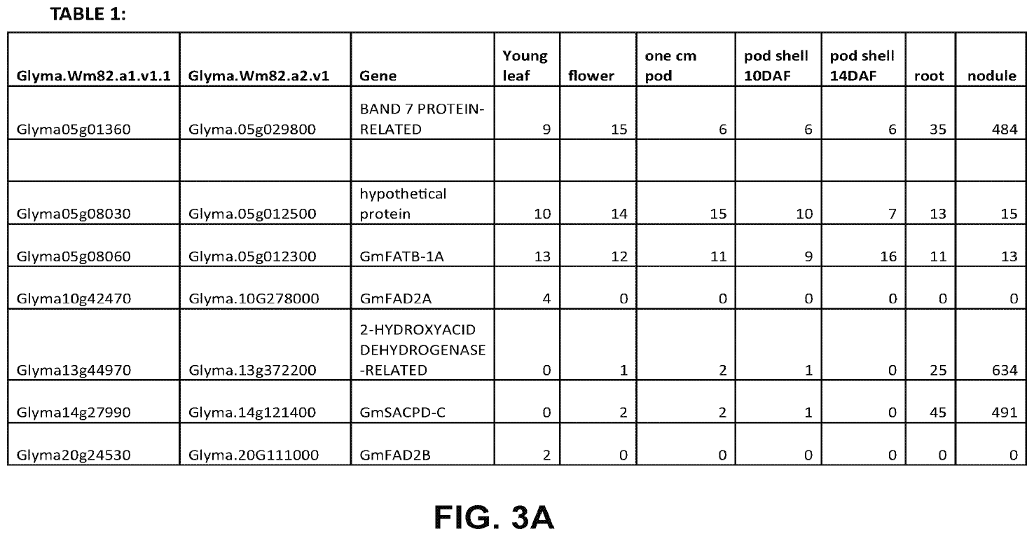

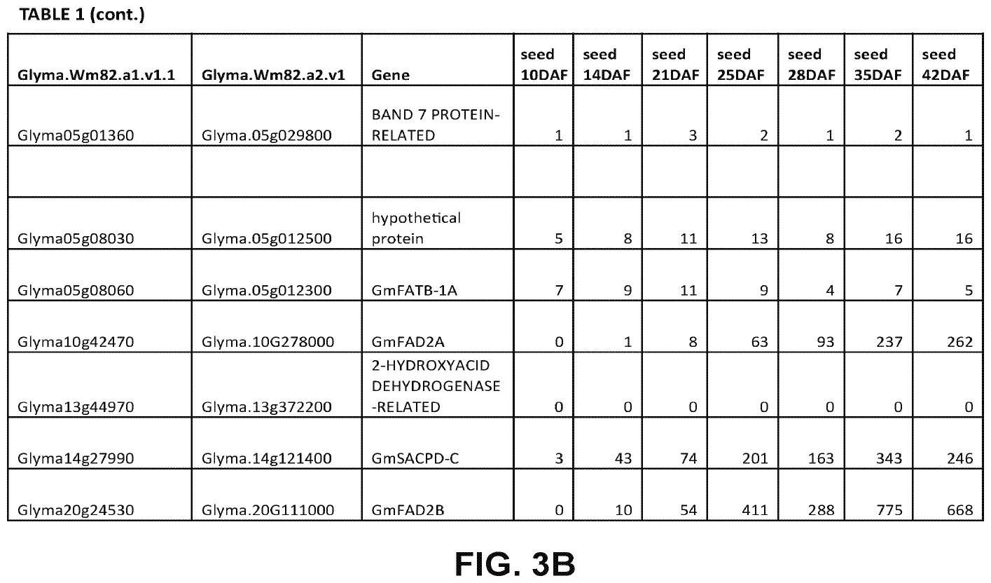

To provide a cis-genic expression cassette that can be used to rescue GmSACPD-C gene expression in root or nodule tissue comprising an inactivated or knocked-out GmSACPD-C gene, the soybean expression database SoyBase was searched to identify two nodule specific genes. Glyma05g01360 and Glyma13g44970 were selected as candidates based on their expression profiles. Both genes are highly expressed in nodules and roots like SACPD-C, while exhibiting no or very low level expression in developing seeds (FIGS. 1 and 3A-B). SEQ ID NOs: 2-5 show the respective promoter and terminator sequences for Glyma05g01360 and Glyma13g44970. Linear cis-genic cassettes comprising a nodule promoter operably linked to GmSACPD-C are cloned and introduced to soybean plants using biolistic methods.

To provide seed-specific knock-out of GmSACPD-C, Geminiviruses are designed to replace the endogenous GmSACPD-C promoter with a nodule specific promoter. The constructs are delivered to soybean plants via Agrobacterium mediated transformation.

IV. Modulating Expression of GmFATB-1A

Overexpression of the GmFATB-1A gene in developing seeds is achieved using one or more of the following methods:

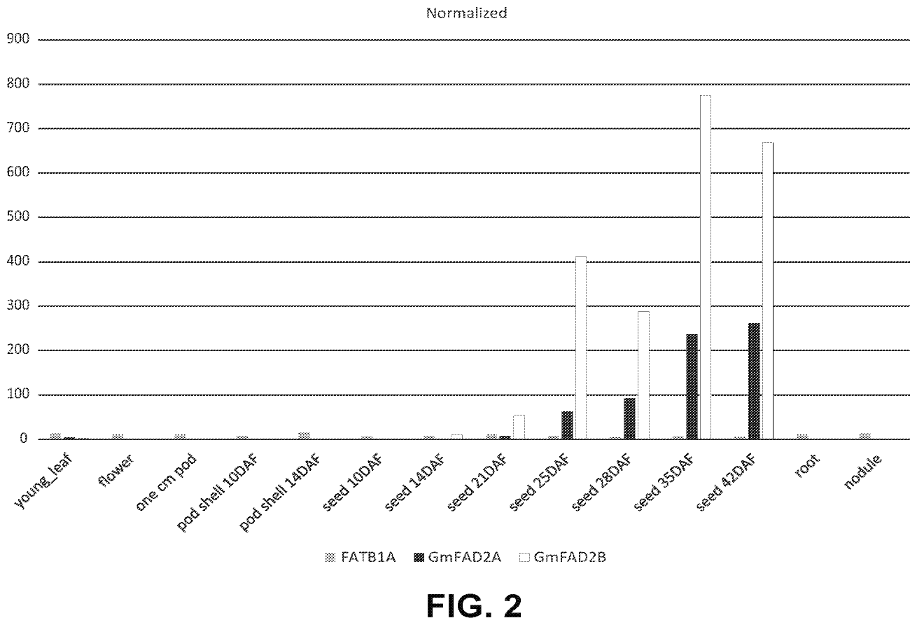

Two seed specific genes GmFAD2A (Glyma10g42470) and GmFAD2B (Glyma20g24530) were selected as candidates for the seed promoter based on expression profiles (i.e., highly expressed in developing seeds with no or very low level expression in other tissue (FIGS. 2 and 3A-B). SEQ ID No. 6 shows the coding sequence of GmFATB-1A. SEQ IDs 7-10 show the promoter and terminator sequences for GmFAD2A (Glyma10g42470) and GmFAD2B (Glyma20g24530).

To provide enhanced expression of GmFATB-1A, a linear cis-genic cassette is synthesized comprising a seed promoter operably linked to GmFATB-1A. The cis-genic cassette is introduced to a soybean plant together with a linear or circular selection marker gene via biolistic co-delivery.

In parallel, to provide a seed specific promoter knock-in to drive overexpression of the FATB-1A gene, a geminivirus is designed to replace the endogenous GmFATB-1A promoter with the promoter sequence that drives expression of endogenous GmFAD2A or GmFAD2B. The coding sequence of GmFAD2A and GmFAD2B is provided in SEQ ID NO: 11 and 12, respectively. TALENs targeting GmFAD2A (or GmFAD2B) upstream of the endogenous FATB-1A gene locus are designed on the first exon. The constructs are delivered to soybean plants via Agrobacterium mediated transformation.

V. Modulating Expression of both GmSACPD-C and GmFATB-1A (Approach 2A)

The techniques for TALEN-mediated knock-out of gmSACPD-C are combined with the introduction of a linear cis-genic cassette having a Glyma13g44970 promoter operably linked to a GmSACPD-C coding sequence with the Glyma13g44970 terminator and a linear cis-genic cassette having a FAD2A promoter operably linked to GmFATB-1A with the FAD2A terminator.

VI. Fatty Acid Composition Analysis

Fatty acid content is analyzed from seed of soybean lines transformed with one or more of the constructs above. One to five seeds of each of the knock-out, cis-genic and control soybean lines are ground for oil extraction. Oil from ground soybean seed is extracted and derivatized to methyl esters. The resulting fatty acid methyl esters are extracted in hexane and resolved by gas chromatography (GC).

The results of the fatty acid compositional analysis from seed oil show stearate (C18:0) levels and/or palmitate levels (C16:0) are significantly increased over the levels obtained from the seed oil of non-transformed control plants. The total saturated fatty acid levels are increased to about 20-40%.

VII. Reduced Expression of SACPD-C by Targeted Mutation (Approach 1A)

Following verification that TAL effector endonucleases created targeted modifications at endogenous target sites, experiments were conducted to create soybean plants with mutations in GmSACPD-C. To accomplish this, TAL effector endonuclease pairs were cloned into a bacterial vector, and delivered to plant cells by Agrobacterium-mediated transformation or by using biolistics.

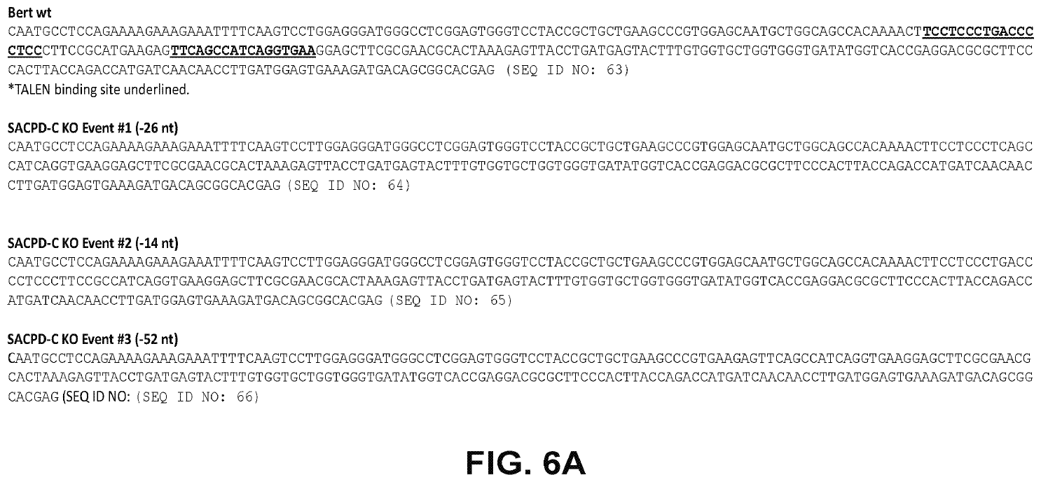

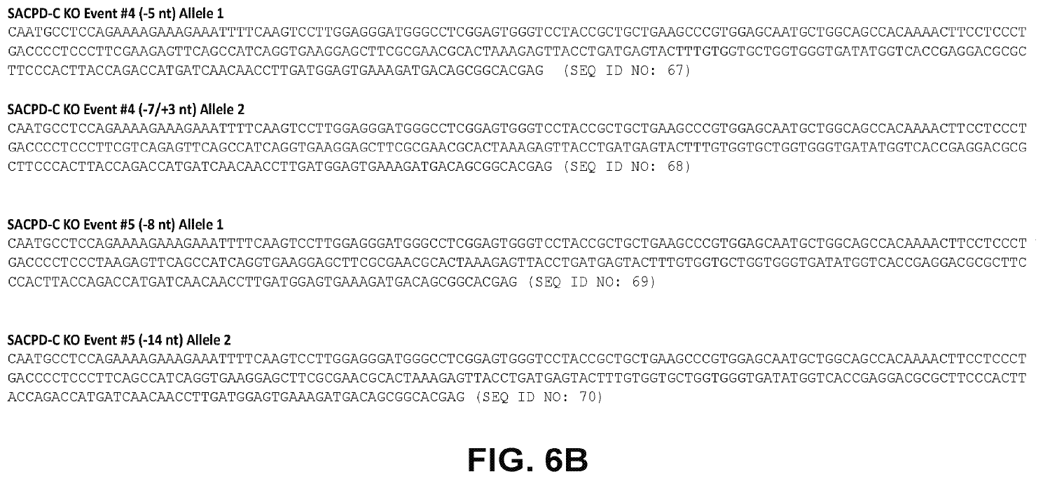

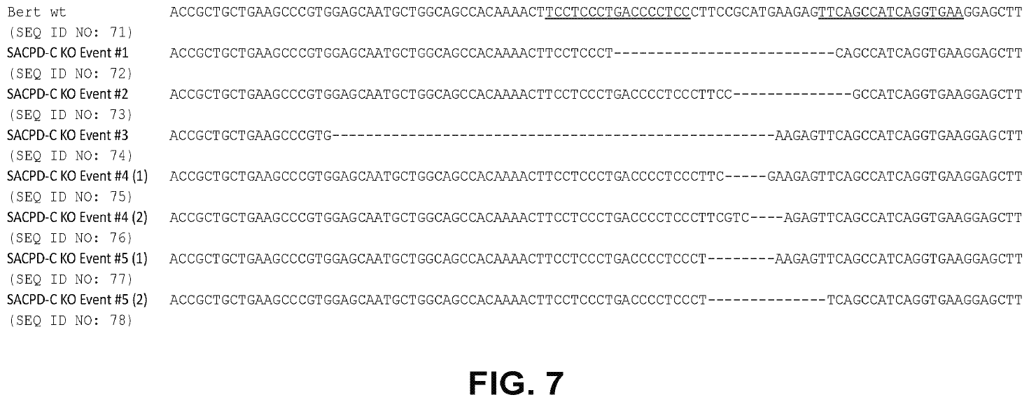

Transgenic soybean plants expressing the TAL effector endonucleases were generated using standard transformation protocols. Following transformation of soybean (cv Bert) with sequences encoding the GmSACPD-C-T03 TAL effector endonuclease, putatively transgenic plants were regenerated. The plants were transferred to soil, and after approximately 4 weeks of growth, a small leaf was collected from each plant for DNA extraction and genotyping. From independent transformations, events #1-#5 with biallelic or homozygous mutations at the target site were generated. DNA samples were analyzed by next generation sequencing of the DNA sequence of GmSACPD-C flanking the GmSACPD-C-T03 TAL effector endonuclease binding site. The resulting reads were then aligned to the wild-type sequence to determine allele types. The results are summarized in TABLE 4 and representative sequences SEQ ID NOS: 63-71 are shown in FIGS. 6A-B and 7. Together, these results confirmed the successful mutagenesis of GmSACPD-C within T₀ soybean plants, with TAL effector endonuclease GmSACPD-C-T03.

TABLE 4
SACPD-C Mutagenesis Summary

SACPD mutations

| Plant ID | allele 1 | allele 2 |
|---|---|---|
| Bert wt | wt | wt |
| Event #1 | −26 | −26 |
| Event #2 | −14 | −14 |
| Event #3 | −52 | −52 |
| Event #4 | −5 | −7, +3 |
| Event #5 | −8 | −14 |

VII. Seed Specific Silencing of SACPD-C by Targeted Promoter Replacement (Approach 1B)

Genome engineering reagents for replacing the endogenous SACPD-C promoter with a nodule specific promoter were delivered to soybean protoplasts. Protoplasts were prepared using conventional methods. Briefly, soybean seeds were grown in vitro and on germination medium under sterile conditions five days prior to transformation. The first true leaves and hypocotyl of the soybean seedling were then digested overnight. Isolation and transformation occurred the day after overnight digestion. During isolation, protoplasts were first screened to ensure a proper yield of one million cells. These cells underwent several washes in a washing buffer solution and were divided into 200,000 cells for each construct used for either validation through a yellow fluorescent protein (YFP) cassette or extraction of its genomic DNA.

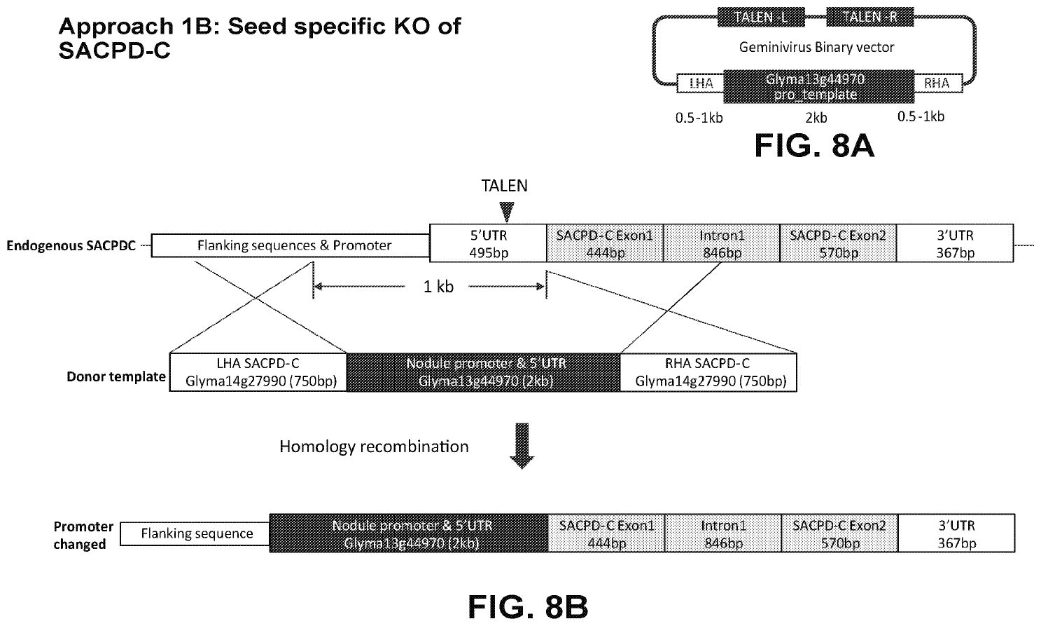

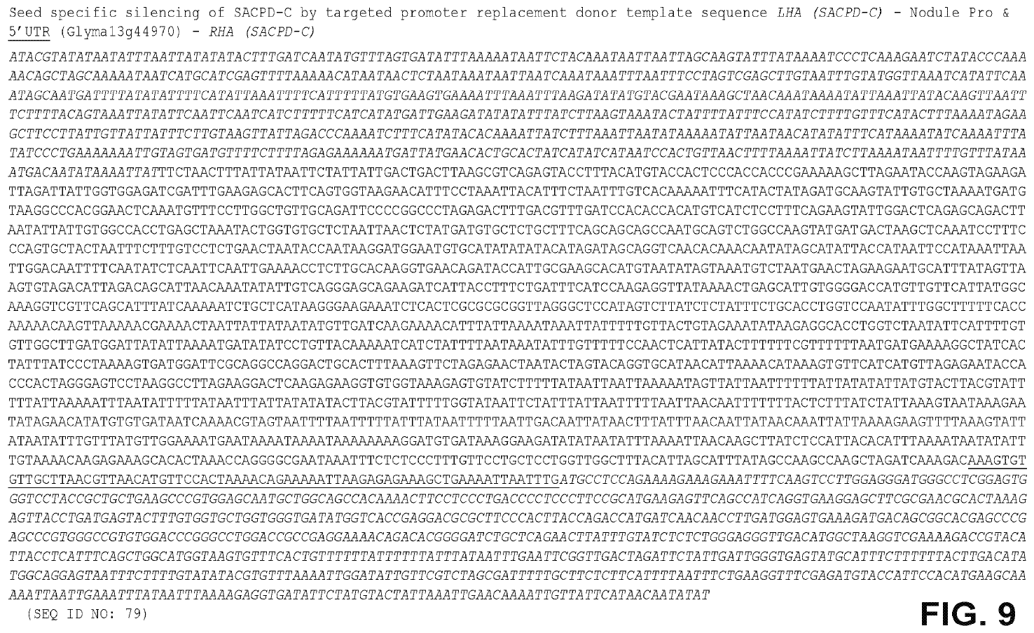

A geminivirus binary vector for targeted promoter replacement was constructed (shown schematically in FIG. 8A). Protoplast cells were transformed via polyethylene glycol in a suspended culture of MMg buffer solution with the plasmid encoding each TALEN pair (“GmSACPD-C-T10” SEQ ID NOS: 42 and 43), along with the geminivirus donor molecule (SEQ ID NO: 79). FIG. 8B illustrates the targeted replacement event.

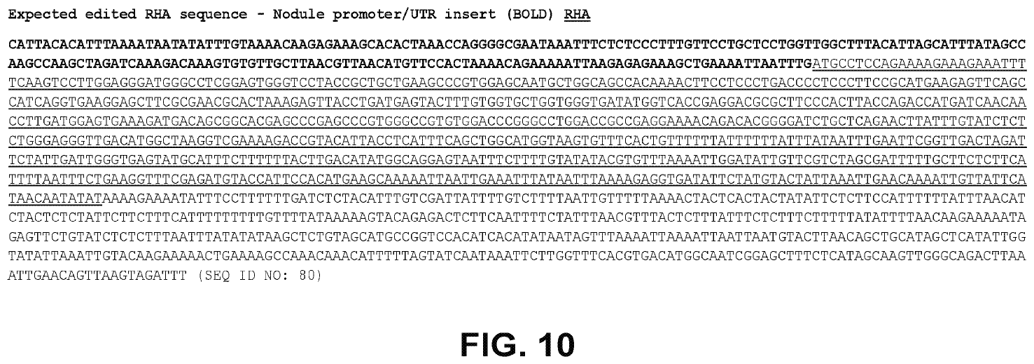

Gene targeting and successful insertion of the donor molecule were detected molecularly by extracting genomic DNA from protoplasts and performing PCR to amplify each homology arm of the insert DNA containing the nodule promoter. Two primer pairs were designed to amplify each homology arm. For each pair, one primer binds to the genomic DNA outside of the homology arm and the other binds to the newly inserted DNA, in this case the nodule promoter. The expected DNA band lengths amplified from these PCR reactions were 1144 base pairs for the left homology arm (LHA) and 1171 base pairs for the right homology arm (RHA). Gel electrophoresis confirmed targeted editing at the SACPD-C site. The expected sequence of the RHA sequence is shown in FIG. 10 (SEQ ID NO: 80).

VIII. Seed Specific Upregulation of FATB-1A by Targeted Promoter Replacement (Approach 1C)

Genome engineering reagents for FATB-1A were delivered to soybean protoplasts. Here, soybean seeds were grown in vitro and on germination medium under sterile conditions five days prior to transformation. The first true leaves and hypocotyl of the soybean seedling were then digested overnight. Isolation and transformation occur the day after overnight digestion. During isolation, protoplasts are first screened to ensure a proper yield of one million cells. These cells undergo several washes in a washing buffer solution and divided into 200,000 cells for each construct used to either be validated through a yellow fluorescent protein (YFP) cassette or to be extracted for its genomic DNA.

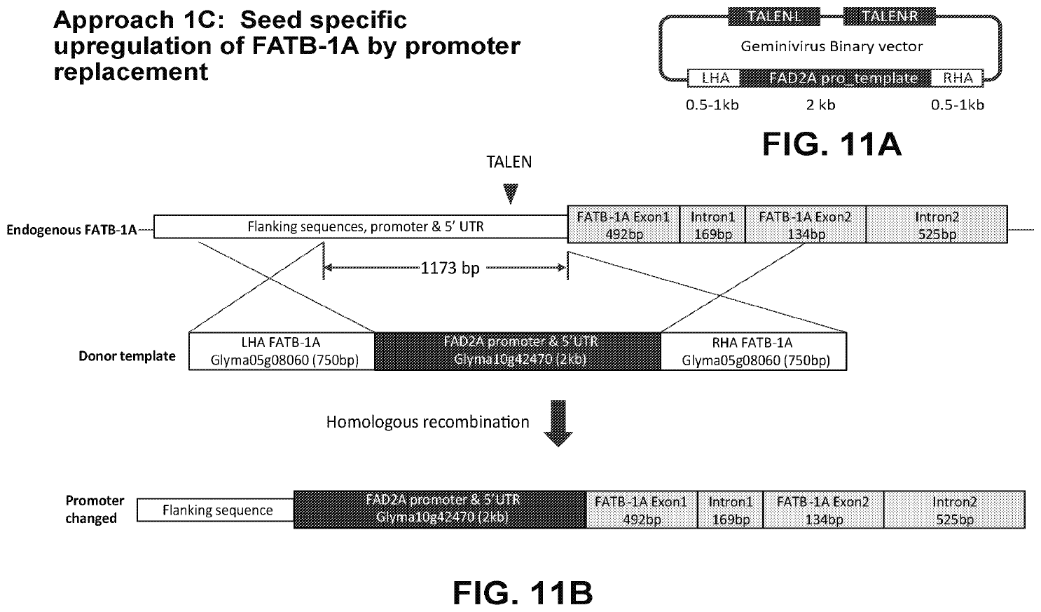

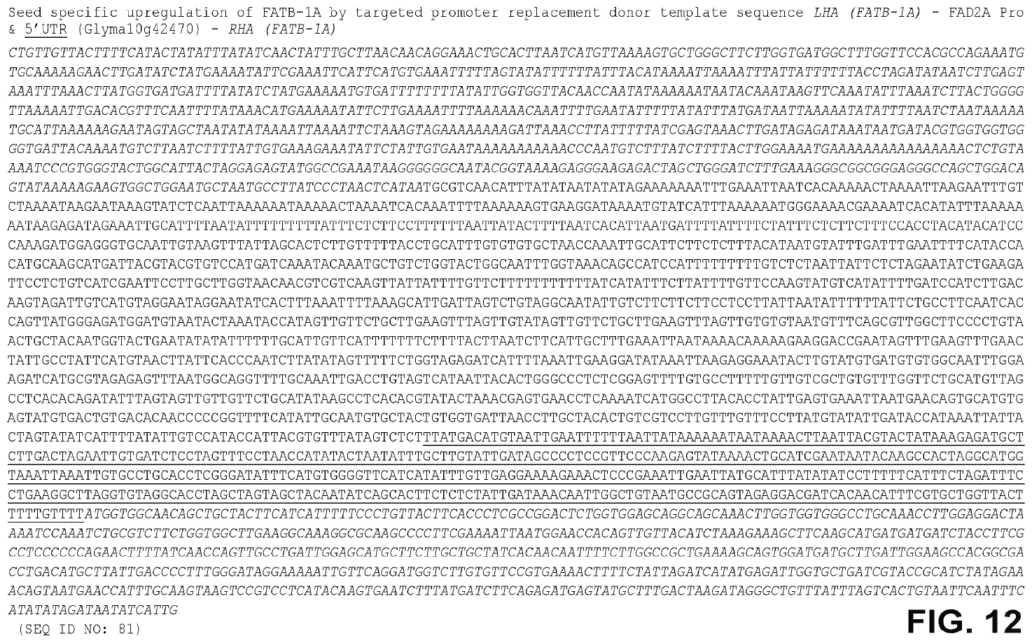

Geminivirus binary vectors for targeted promoter replacement were constructed (shown schematically in FIG. 11A) with TALEN pairs “GmFATB1A-T2” SEQ ID NOS: 52 and 53, “GmFATB1A-T3” SEQ ID NOS: 55 and 56, “GmFATB1A-T4” SEQ ID NOS: 58 and 59, respectively. Protoplast cells were transformed via polyethylene glycol in a suspended culture of MMg buffer solution with the plasmid encoding each TALEN pair, along with geminivirus donor molecule (SEQ ID NO: 81). FIG. 11B illustrates the targeted replacement event. Gene targeting and successful insertion of the donor molecules were detected molecularly by extracting genomic DNA from protoplasts and performing PCR to amplify each homology arm of the insert DNA containing the FAD2A promoter. Two primer pairs were designed to amplify each homology arm. For each pair, one primer binds to the genomic DNA outside of the homology arm and the other binds to the newly inserted DNA, in this case the FAD2A promoter. The expected DNA band length amplified from these PCR reactions were 1365 base pairs for the LHA and 1430 base pairs for the RHA. Gel electrophoresis confirmed targeted editing at the FATB-1A site. Based on the activities of the TALEN pairs, SEQ ID NOS: 55 and 56 were selected for advancement.

IX. Seed Specific Upregulation of FATB-1A and Nodule/Leaf Specific Expression of SACPD-C by Cis-Genic Cassette (Approach 2A)

A linear cis-genetic construct for tissue specific expression of SACPD-C and FATB-1A genes is constructed having the sequence set forth in SEQ ID NO: 82.

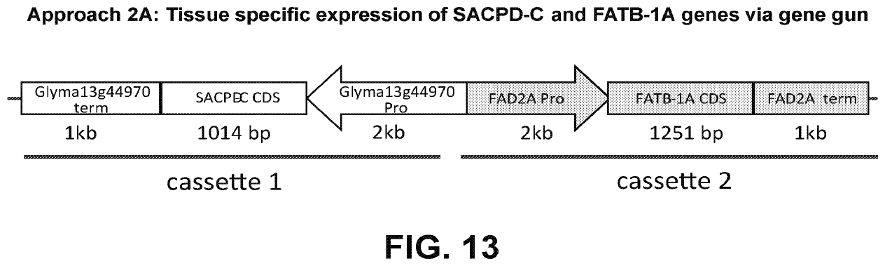

Immature cotyledons are excised from immature soybean pods and grown in liquid cultures on a shaker until soy somatic embryogenic calli form (4-8 weeks). Soy somatic embryogenic calli are co-bombarded with gold particles coated with the DNA construct with cassettes 1 and 2 (FIG. 13) and a selectable marker. After a week of resting in regeneration media, the selection agent is added. The selection media is replaced weekly for approximately 4 weeks. Then the transformed embryogenic calli is broken up into 1-2 mm pieces and placed on a charcoal-rich maturation media for 4-8 weeks. The transformed mature embryos are desiccated before moving to rooting media.

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. Various features and aspects of the disclosed embodiments can be combined with or substituted for one another in order to form various embodiments. Thus, it is intended that the scope of at least some of the present disclosure should not be limited by the embodiments described herein.

The scope of this disclosure should be determined by the appended claims and their legal equivalents. The scope of the present disclosure encompasses other embodiments which may become obvious to those skilled in the art. Thus, the scope of the present disclosure is to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address every problem sought to be solved by the present disclosure for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto.

Various examples have been described. These and other examples are within the scope of the following claims.

SEQUENCE LISTING

1) GmSACPD-C coding sequence
(SEQ ID NO: 1)
ATGCCTCCAGAAAAGAAAGAAATTTTCAAGTCCTTGGAGGGATGGGCCT
CGGAGTGGGTCCTACCGCTGCTGAAGCCCGTGGAGCAATGCTGGCAGCC
ACAAAACTTCCTCCCTGACCCCTCCCTTCCGCATGAAGAGTTCAGCCAT
CAGGTGAAGGAGCTTCGCGAACGCACTAAAGAGTTACCTGATGAGTACT
TTGTGGTGCTGGTGGGTGATATGGTCACCGAGGACGCGCTTCCCACTTA
CCAGACCATGATCAACAACCTTGATGGAGTGAAAGATGACAGCGGCACG
AGCCCGAGCCCGTGGGCCGTGTGGACCCGGGCCTGGACCGCCGAGGAAA
ACAGACACGGGGATCTGCTCAGAACTTATTTGTATCTCTCTGGGAGGGT
TGACATGGCTAAGGTCGAAAAGACCGTACATTACCTCATTTCAGCTGGC
ATGGACCCTGGGACAGACAACAACCCATATTTGGGGTTTGTGTACACGT
CATTCCAAGAGCGAGCAACATTTGTGGCGCACGGGAACACGGCTCGGCT
CGCGAAGGAGGGCGGGGATCCAGTGCTGGCGCGCCTATGCGGGACCATC
GCAGCGGACGAGAAGCGGCACGAGAACGCGTACTCAAGAATCGTGGAGA
AGCTTCTGGAAGTGGACCCCACCGGGGCAATGGTGGCCATAGGGAACAT
GATGGAGAAGAAGATCACGATGCCGGCGCACCTTATGTACGATGGGGAT
GACCCCAGGCTATTCGAGCACTACTCCGCTGTGGCGCAGCGCATAGGCG
TGTACACCGCCAACGACTACGCAGACATCTTGGAGTTTCTCGTTGAACG
GTGGAGATTGGAGAAGCTTGAAGGATTGATGGCTGAGGGGAAGCGGGCG
CAGGATTTCGTGTGTGGGTTGGCGCCGAGGATTAGGAGGTTGCAAGAGC
GCGCTGATGAGCGAGCGCGTAAGATGAAGAAGCATCATGGCGTTAAGTT
CAGTTGGATTTTCAATAAAGAATTGCTTTTGTGA
2) Nodule specific Glyma05g01360 promoter
sequence 2KB
(SEQ ID NO: 2)
CTAAAATGAAATTAAAATATAAAATATATAAATTAAAAATATCATTTTT
AAATTATAAAGAAAAAAAATCACTTTTAAAATTATAAAGACTAAAAAAA
ATTAAAATATAAAGGGAAAAAATTCAATTCCAAACGATAAAAAAACAAA
AAAAAAATATCTTCAAACTATAGAAATAATTTAAGTTCCCGAATTTGAA
TCTAGTTAAGTTGAAATAAAAAATAAAAATTTATATCAAATTTGATTCA
GCTCCCAGAAGAGGCAGTATATAATTTCATATACACCAAGCGCCAAGAC
GCAAGCCGTTAGTTACGAGGTCCTTTTCCGTTTGTCAGTATCAAACCCC
TTTTTTAATTTCATTTTCTTATTTTTTGGCATAAAAAATTTACAGCAGC
GAGAGATAAATCCCATGCCACTCTCCAGCGAGAGATATACTTAATTTGA
ACATTTGTGTGTACTTTAAATGCAAGAAGTGATAATGTCTTGATCAAAA
ATAAAAATGTAAGAAGTGATAACTCCCCAGCCCATAACCAGAGTTTGAT
TTCTAATTATACCAATAAAACAATACTTATAGGTAAAGGAATTGTTGTT
AACTAATACTTTACTTTTTAAAATTATAAAAATATTTCTGAATTTAATC
TTAGATCTTAAAAAATGAAAAAAAAACTATAAAATAATGTTTTATTGTT
AGTATTTTAAAATTATAATATTATATTTTATAATAATAATAATAATTAG
AAAAAAACCGCTCTTAAGTCATTTAACAATAATAATAATGTTTTATTAT
TATTATTATTTCTGATTTGACTATGGTTGAATAAATTACTATATTGATA
AATCTCTTATTGAGAAAAAAAAACCATTCTTAAGTCATTTAACAATAAG
AATATTTCAAATATTAATAAAAAAAAACCAAACATCAATTAAGAAACTG
AATGTTTATTTTTAATAACTAAAATTTAAATAATAAATACTTTTGATTA
TATAAGCTATATTATAATCTAAATTTATAATACAAATAATTTTAAATAT
ATAATTTATATTTAATTTATGTAATATATATAAATATATTCTAAAATTT
ATAATAAAAAAGTTATAATATAATATAAAATTATTTTAAATTACTAATA
ACTTCTTTTAAATATGTTAAATTTTAATTTAAAAATAGAGATGAGAATG
ATAAAATATAAGAGATATAAACAGTTAAAAAATTTCTTAAAATTATTCT
AGTCGAATATTATTATTCTAATTTATATTTATAGAAAGATTATCATACC
AAAAAAATTGCTCAAAAAAATTATTATACCAAAAAAATATATATAGAAA
AATTATTATCACATCCATGTCAAGTTCACGGTTTTGTCGGGGATGGTGG
ATTGGTTTAGACTTATAAAGTCGTCTTTTGCTGGCCAGCACTTCGTAAA
TCCACTTATCTGAAATTGTGAACAGTATTGTTCTTGCGTTGTTTCGGAG
GGTCAATTGTTTGGTTGAGCAAAAATCTGAAATTCAATTTCAGGTCTTT
AACTTTTAACTTTCTTGGTTGATATCTGGAGGGATTGCTTCCAAAACTC
TGCATTGTCTGTGTGTAATGGTTGTTAGAGTTTACCCAATTGGCTAATT
ATTTATATATTTTTATGTTTGGACTTTGTCTTTGTGTTCCCTTGTTCCA
CACATAAATTAAACAAAGGTACTATATACTACTTTTCTAGGTTGTATCT
GCATGACAACAATGATATCTCCTTGAAGAGGTTGTGATTGGCTTGTCAT
GCCAGTTAGCTTGCAATTCTGGGTTTCTGACAGACAGTATTTAAATTGT
ATTTATGTATATGACTATGACTTTGGAATGTATACGGCTAAAACATATA
CGTCATGATTAATGGTGGTGATGAATATATCAAGAATAGCAAGTATTTG
TGTACTCTCAGTATTCGAGAGCTACTTGCTTATAATTTATGCAAGAACT
CTCCTAAGTCAGCTTTGCTTTCGAAGCCTTTATCTTTGAG
3) Nodule specific Glymal3g44970 promoter
sequence 2KB
(SEQ ID NO: 3)
TTCTAACTTTATTATAATTCTATTATTGACTGACTTAAGCGTCAGAGTA
CCTTTACATGTACCACTCCCACCACCCGAAAAAGCTTAGAATACCAAGT
AGAAGATTAGATTATTGGTGGAGATCGATTTGAAGAGCACTTCAGTGGT
AAGAACATTTCCTAAATTACATTTCTAATTTGTCACAAAAATTTCATAC
TATAGATGCAAGTATTGTGCTAAAATGATGTAAGGCCCACGGAACTCAA
ATGTTTCCTTGGCTGTTGCAGATTCCCCGGCCCTAGAGACTTTGACGTT
TGATCCACACCACATGTCATCTCCTTTCAGAAGTATTGGACTCAGAGCA
GACTTAATATTATTGTGGCCACCTGAGCTAAATACTGGTGTGCTCTAAT
TAACTCTATGATGTGCTCTGCTTTCAGCAGCAGCCAATGCAGTCTGGCC
AAGTATGATGACTAAGCTCAAATCCTTTCCCAGTGCTACTAATTTCTTT
GTCCTCTGAACTAATACCAATAAGGATGGAATGTGCATATATATACATA
GATAGCAGGTCAACACAAACAATATAGCATATTACCATAATTCCATAAA
TTAATTGGACAATTTTCAATATCTCAATTCAATTGAAAACCTCTTGCAC
AAGGTGAACAGATACCATTGCGAAGCACATGTAATATAGTAAATGTCTA
ATGAACTAGAAGAATGCATTTATAGTTAAGTGTAGACATTAGACAGCAT
TAACAAATATATTGTCAGGGAGCAGAAGATCATTACCTTTCTGATTTCA
TCCAAGAGGTTATAAAACTGAGCATTGTGGGGACCATGTTGTTCATTAT
GGCAAAGGTCGTTCAGCATTTATCAAAAATCTGCTCATAAGGGAAGAAA
TCTCACTCGCGCGCGGTTAGGGCTCCATAGTCTTATCTCTATTTCTGCA
CCTGGTCCAATATTTGGCTTTTTCACCAAAAACAAGTTAAAAACGAAAA
CTAATTATTATAATATGTTGATCAAGAAAACATTTATTAAAATAAATTA
TTTTTGTTACTGTAGAAATATAAGAGGCACCTGGTCTAATATTCATTTT
GTGTTGGCTTGATGGATTATATTAAAATGATATATCCTGTTACAAAAAT
CATCTATTTTAATAAATATTTGTTTTTCCAACTCATTATACTTTTTTCG
TTTTTTAATGATGAAAAGGCTATCACTATTTATCCCTAAAAGTGATGGA
TTCGCAGGCCAGGACTGCACTTTAAAGTTCTAGAGAACTAATACTAGTA
CAGGTGCATAACATTAAAACATAAAGTGTTCATCATGTTAGAGAATACC
ACCCACTAGGGAGTCCTAAGGCCTTAGAAGGACTCAAGAGAAGGTGTGG
TAAAGAGTGTATCTTTTTATAATTAATTAAAAATAGTTATTAATTTTTT
ATTATATATTATGTACTTACGTATTTTTATTAAAAATTTAATATTTTTA
TAATTTATTATATATACTTACGTATTTTTGGTATAATTCTATTTATTAA
TTTTTAATTAACAATTTTTTTACTCTTTATCTATTAAAGTAATAAAGAA
TATAGAACATATGTGTGATAATCAAAACGTAGTAATTTTAATTTTTATT
TATAATTTTTAATTGACAATTATAACTTTATTTAACAATTATAACAAAT
TATTAAAAGAAGTTTTAAAGTATTATAATATTTGTTTATGTTGGAAAAT
GAATAAAATAAAATAAAAAAAAGGATGTGATAAAGGAAGATATATAATA
TTTAAAATTAACAAGCTTATCTCCATTACACATTTAAAATAATATATTT
GTAAAACAAGAGAAAGCACACTAAACCAGGGGCGAATAAATTTCTCTCC
CTTTGTTCCTGCTCCTGGTTGGCTTTACATTAGCATTTATAGCCAAGCC
AAGCTAGATCAAAGACAAAGTGTGTTGCTTAACGTTAACATGTTCCACT
AAAACAGAAAAATTAAGAGAGAAAGCTGAAAATTAATTTG
4) Nodule specific Glyma05g01360 terminator
sequence 903bp
(SEQ ID NO: 4)
CCTCTACTCCACCTAGATCTTGTATTTGGTTTGTATGGGAGTATGTTTG
AAGCTATAGCGCCTGTGGTTGTATACCTGTATTTCTGTGCAGTGTGTTT
TGTGATTTTGTTTTAGAATAAACTGCAAAATTGATCTTCCAAAGATTAT
GGCATCACCTCATTAATGTTTTTAAGATTTTTGTTATCAAATTAGTCCC
ACAAATATCTAAAATGTTACCACATTTGTTCACATAGAGGACTAAAGAA
GTGGTAACACAAAATAATTTAGTATTTGATTTGTATCTCTGTGGGATTG
ATTTGTTGACCAAAATCTTTGGAGGACCAGTTCAAGGACTTACTCTATC
GATTTATACTTTCAGTTTCAGGCAGTCAAGTAATACTGTATATTCTGTT
GTCTATATTGTGGATCATGCATAACTAAACTATCAAGTATCCCTGTATA
TACAAGTTGTCTAAATTGTGAAAGTTACATATACAAGAAATTTCCTATA
TACCGTTAGAGAGTAGATAAACTATATACCGTTAGAGAGTTTTCATGCT
AATTGTTAAATTAAATACTACGCTTTCTAACCTTCCATCTTTTGAACCA
ATAAAAAATGTTTCTACTCATCGTATTCTCCATTAAGTTGAAATATTAC
CAATTAAGTTATGTTACAAAATTTTCCCTCGGAATCTGAAGGTTTGCTT
GTATAGCAGTGCTCATTTTTTTCTAAAAGAAAATATACAATTGCTTCCC
CTTTACGAATAGTAAAGTCATTCACAACGAAGTAAAGAACAGAAGTAAC
ACAGCAGCTACCATCTCATCCAAGAAAAAAAAAAAGTAATTCTGGAGCT
ATTTGTTTCAGATATTCAACTTCTAAAAGAATATTAAACTTAAAACAGG
TAAAAATGATAAACAAATACC
5) Nodule specific Glymal3g44970 terminator
sequence 1KB
(SEQ ID NO: 5)
CTAGCCTTTCAACCATCATTATATGCCGTACGTACGACTCAAATTAAAT
AAATAAACGGCTAGCTCAAGGTTTCTATTTGCTTAATAAATTCTTGTCT
TTTGTTATTATTCTCGTTGCACTCGCACAGTTACTTCCCTGCTTATTGT
TTTAGGAATACAATATAATTCTATATATTTGAGTTTGAGTTGGTGCATA
CGACAAATCGACAATGCCAATTTCTTTTTATACAATAAATTCTTGTATT
TTTCTTTTATAATTACGATTAGGATGTTTTCTCACTAGAGAGCTTTGTA
TAATGTATCAGATTTGCGGATCTACTATTAGGATCGATGTTTACCCAAT
AAAGCATTATATCATGTATGCATAAATAGTTTTGTAAAAAAAAATGTAT
ATTGATCGTTGGTAATGTCACTAACTTTCGCTTGTAACTGTTGTTGGTA
AATTGGTAATGTCGTCACTTACGAATTGGATCTATTAGGAGGTTAAATA
CGGGTCACCTTGTGACAGAGTTGATTGTTCTCAGATCCAGAACTACTAA
TTTGTTGGCTAACCTTAATATCTTATTTATTCATTCCAATCTTTTCCAT
TATCTATATATTCTCCAAGATCTTTTTAGTTAAAGTTGTAAAATCAATT
TTCCTGAAAAACAAAAAAAAAGGCTAATAATACACTTTTTAATATATTA
TTTCTAACACATTTTATTATTAGTTAAAATTTATTAATAACTATAAAAT
TAAGGGAAAAAACCATTAAATAATATATTAAAAAGTATGTTTGAAATCC
CAAACGTGTGAAAGTAGACTTGCTAATGAGGTAAACTTGCTATTTACAT
TTTTCTTACGAGAGGAGATTCAAATAATAATTGTTTAGGGGTGTTGCAT
GTAATTGTAAAGGGGTTGTGTCTCGGAAGGGTTTTTATTCTTTGTTGTC
ATGCAGGTTTGGACACTACTTGTCTTAGAGCTGTATACCTTTCAAAAAA
TAAATAAATTCATTTTTTCT
6) GmFATB-1A coding sequence
(SEQ ID NO: 6)
ATGGTGGCAACAGCTGCTACTTCATCATTTTTCCCTGTTACTTCACCCT
CGCCGGACTCTGGTGGAGCAGGCAGCAAACTTGGTGGTGGGCCTGCAAA
CCTTGGAGGACTAAAATCCAAATCTGCGTCTTCTGGTGGCTTGAAGGCA
AAGGCGCAAGCCCCTTCGAAAATTAATGGAACCACAGTTGTTACATCTA
AAGAAAGCTTCAAGCATGATGATGATCTACCTTCGCCTCCCCCCAGAAC
TTTTATCAACCAGTTGCCTGATTGGAGCATGCTTCTTGCTGCTATCACA
ACAATTTTCTTGGCCGCTGAAAAGCAGTGGATGATGCTTGATTGGAAGC
CACGGCGACCTGACATGCTTATTGACCCCTTTGGGATAGGAAAAATTGT
TCAGGATGGTCTTGTGTTCCGTGAAAACTTTTCTATTAGATCATATGAG
ATTGGTGCTGATCGTACCGCATCTATAGAAACAGTAATGAACCATTTGC
AAGAAACTGCACTTAATCATGTTAAAAGTGCTGGGCTTCTTGGTGATGG
CTTTGGTTCCACGCCAGAAATGTGCAAAAAGAACTTGATATGGGTGGTT
ACTCGGATGCAGGTTGTGGTGGAACGCTATCCTACATGGGGTGACATAG
TTCAAGTGGACACTTGGGTTTCTGGATCAGGGAAGAATGGTATGCGCCG
TGATTGGCTTTTACGTGACTGCAAAACTGGTGAAATCTTGACAAGAGCT
TCCAGTGTTTGGGTCATGATGAATAAGCTAACACGGAGGCTGTCTAAAA
TTCCAGAAGAAGTCAGACAGGAGATAGGATCTTATTTTGTGGATTCTGA
TCCAATTCTGGAAGAGGATAACAGAAAACTGACTAAACTTGACGACAAC
ACAGCGGATTATATTCGTACCGGTTTAAGTCCTAGGTGGAGTGATCTAG
ATATCAATCAGCATGTCAACAATGTGAAGTACATTGGCTGGATTCTGGA
GAGTGCTCCACAGCCAATCTTGGAGAGTCATGAGCTTTCTTCCATGACT
TTAGAGTATAGGAGAGAGTGTGGTAGGGACAGTGTGCTGGATTCCCTGA
CTGCTGTATCTGGGGCCGACATGGGCAATCTAGCTCACAGCGGGCATGT
TGAGTGCAAGCATTTGCTTCGACTGGAAAATGGTGCTGAGATTGTGAGG
GGCAGGACTGAGTGGAGGCCCAAACCTGTGAACAACTTTGGTGTTGTGA
ACCAGGTTCCAGCAGAAAGCACCTAA
7) Seed specific GmFAD2A promoter sequence 2KB
(SEQ ID NO: 7)
TGCGTCAACATTTATATAATATATAGAAAAAAATTTGAAATTAATCACA
AAAACTAAAATTAAGAATTTGTCTAAAATAAGAATAAAGTATCTCAATT
AAAAAATAAAAACTAAAATCACAAATTTTAAAAAAGTGAAGGATAAAAT
GTATCATTTAAAAAATGGGAAAACGAAAATCACATATTTAAAAAAATAA
GAGATAGAAATTGCATTTTAATATTTTTTTTTATTTCTCTTCCTTTTTT
AATTATACTTTTAATCACATTAATGATTTTATTTTCTATTTCTCTTCTT
TCCACCTACATACATCCCAAAGATGGAGGGTGCAATTGTAAGTTTATTA
GCACTCTTGTTTTTACCTGCATTTGTGTGTGCTAACCAAATTGCATTCT
TCTCTTTACATAATGTATTTGATTTGAATTTTCATACCACATGCAAGCA
TGATTACGTACGTGTCCATGATCAAATACAAATGCTGTCTGGTACTGGC
AATTTGGTAAACAGCCATCCATTTTTTTTTGTCTCTAATTATTCTCTAG
AATATCTGAAGATTCCTCTGTCATCGAATTCCTTGCTTGGTAACAACGT
CGTCAAGTTATTATTTTGTTCTTTTTTTTTTTATCATATTTCTTATTTT
GTTCCAAGTATGTCATATTTTGATCCATCTTGACAAGTAGATTGTCATG
TAGGAATAGGAATATCACTTTAAATTTTAAAGCATTGATTAGTCTGTAG
GCAATATTGTCTTCTTCTTCCTCCTTATTAATATTTTTTATTCTGCCTT
CAATCACCAGTTATGGGAGATGGATGTAATACTAAATACCATAGTTGTT
CTGCTTGAAGTTTAGTTGTATAGTTGTTCTGCTTGAAGTTTAGTTGTGT
GTAATGTTTCAGCGTTGGCTTCCCCTGTAACTGCTACAATGGTACTGAA
TATATATTTTTTGCATTGTTCATTTTTTTCTTTTACTTAATCTTCATTG
CTTTGAAATTAATAAAACAAAAAGAAGGACCGAATAGTTTGAAGTTTGA
ACTATTGCCTATTCATGTAACTTATTCACCCAATCTTATATAGTTTTTC
TGGTAGAGATCATTTTAAATTGAAGGATATAAATTAAGAGGAAATACTT
GTATGTGATGTGTGGCAATTTGGAAGATCATGCGTAGAGAGTTTAATGG
CAGGTTTTGCAAATTGACCTGTAGTCATAATTACACTGGGCCCTCTCGG
AGTTTTGTGCCTTTTTGTTGTCGCTGTGTTTGGTTCTGCATGTTAGCCT
CACACAGATATTTAGTAGTTGTTGTTCTGCATATAAGCCTCACACGTAT
ACTAAACGAGTGAACCTCAAAATCATGGCCTTACACCTATTGAGTGAAA
TTAATGAACAGTGCATGTGAGTATGTGACTGTGACACAACCCCCGGTTT
TCATATTGCAATGTGCTACTGTGGTGATTAACCTTGCTACACTGTCGTC
CTTGTTTGTTTCCTTATGTATATTGATACCATAAATTATTACTAGTATA
TCATTTTATATTGTCCATACCATTACGTGTTTATAGTCTCTTTATGACA
TGTAATTGAATTTTTTAATTATAAAAAATAATAAAACTTAATTACGTAC
TATAAAGAGATGCTCTTGACTAGAATTGTGATCTCCTAGTTTCCTAACC
ATATACTAATATTTGCTTGTATTGATAGCCCCTCCGTTCCCAAGAGTAT
AAAACTGCATCGAATAATACAAGCCACTAGGCATGGTAAATTAAATTGT
GCCTGCACCTCGGGATATTTCATGTGGGGTTCATCATATTTGTTGAGGA
AAAGAAACTCCCGAAATTGAATTATGCATTTATATATCCTTTTTCATTT
CTAGATTTCCTGAAGGCTTAGGTGTAGGCACCTAGCTAGTAGCTACAAT
ATCAGCACTTCTCTCTATTGATAAACAATTGGCTGTAATGCCGCAGTAG
AGGACGATCACAACATTTCGTGCTGGTTACTTTTTGTTTT
8) Seed specific GmFAD2B promoter sequence 2KB
(SEQ ID NO: 8)
AACATATTGGGGGTACCAAACAATTTGCACCCCATAATAAGGAACTGTG
GACAAAATTGCATTTGCCACACCTCCCAATTTATTTAGTAGACGACTCT
TCCAAGTTCCAAGAAGCTAACCTTGAGTTTATTTGCTCCTCAATGTGAT
TGAAGTCAGCTCTCTGAATTGCTTCATGCACAATCTTCAACTTCGTGTT
TTTTTAGTTACCATATGAATCTCCCAATTTATTTAGATTGTACAATAAA
TTGAGAAAACTCAAATAATTAAATCCAATAATTTTTTCTTAAAAATCAT
TTCAATCTAATTCACTGTGAACACCTTTATCTAAAATTTTACATGAAAT
TTCAAATTTAATTCAATTCTTACTAACATAATAATGCTGGATATTTTCT
TAATTCCAAATAATACTATTAAATATTAGATTCAATGTTTATAATATAT
TCCACAATTGTATTTTTTTTATTTGTAAGAATTAAAAATAAATATCAAA
AAGTTTACAACACTCACATATCCTAACCAATCAATTGAGTTAAATTCCT
TATATAGGGATTGTATGAATTTTCATTTTAATACATATATTTAAAATCT
TATTATTGCAAAAAATAATAATCTAATACTTTTTTTTAAAATACATGAA
TGTATGACCATGAAAAATGGCACCATGTTAAAAAAAACTGTTTTAAATA
TGAATATTTTCTCTCATAATTAATATTTTTTCTATGCAGTATTGTTTAA
AAAAAAAAACTGTTTCTTCAATTTCTGAAACACCGAAAAAGAGAGAAAG
AAAAAATTATTGTTTTTTAATTTAATATGGTGTGCTTACTCACAAAGCA
GTCTTACACTAATCTCGAAATAAACTTATCAGATGGTCGAAAATCCTTT
GGCACGTTAAAACACGTCGTACAAAAGATGCAGCTGATCTGATTCTCCA
CTTGTCTCAAGCAGACATCACACTGATCACAGGTGGAAACCAAATTTGC
CTAAGTTCCAAGGCCTCGGTGTGACTCAGCCCCAAGTGACGCCAACCAA
ACGCGTCCTAACTAAGGTGTAGAAGAAACAGATAGTATATAAGTATACC
ATATAAGAGGAGAGTGAGTGGAGAAGCACTTCTCCTTTTTTTTTTCTCT
GTTGAAATTGAAAGTGTTTTCCGGGAAATAAATAAAATAAATTAAAATC
TTACACACTCTAGGTAGGTACTTCTAATTTAATCCACACTTTGACTCTA
TATATGTTTTAAAAATAATTATAATGCGTACTTACTTTCTCATTATACT
AAATTTAACATCGATGATTTTATTTTCTGTTTCTCTTCTTTCCACCTAC
ATACATCCCAAAATTTAGGGTGCAATTTTAAGTTTATTAACACATGTTT
TTAGCTGCATGCTGCCTTTGTGTGTGCTCACCAAATTGCATTCTTCTCT
TTATATGTTGTATTTGAATTTTCACACCATATGTAAACAAGATTACGTA
CGTGTCCATGATCAAATACAAATGCTGTCTTATACTGGCAATTTGATAA
ACAGCCGTCCATTTTTTCTTTTTCTCTTTAACTATATATGCTCTAGAAT
CTCTGAAGATTCCTCTGCCATCGAATTTCTTTCTTGGTAACAACGTCGT
CGTTATGTTATTATTTTATTCTATTTTTATTTTATCATATATATTTCTT
ATTTTGTTCGAAGTATGTCATATTTTGATCGTGACAATTAGATTGTCAT
GTAGGAGTAGGAATATCACTTTAAAACATTGATTAGTCTGTAGGCAATA
TTGTCTTCTTTTTCCTCCTTTATTAATATATTTTGTCGAAGTTTTACCA
CAAGGTTGATTCGCTTTTTTTGTCCCTTTCTCTTGTTCTTTTTACCTCA
GGTATTTTAGTCTTTCATGGATTATAAGATCACTGAGAAGTGTATGCAT
GTAATACTAAGCACCATAGCTGTTCTGCTTGAATTTATTTGTGTGTAAA
TTGTAATGTTTCAGCGTTGGCTTTCCCTGTAGCTGCTACA
9) Seed specific GmFAD2A terminator sequence 1KB
(SEQ ID NO: 9)
TGGAGCAACCAATGGGCCATAGTGGGAGTTATGGAAGTTTTGTCATGTA
TTAGTACATAATTAGTAGAATGTTATAAATAAGTGGATTTGCCGCGTAA
TGACTTTGTGTGTATTGTGAAACAGCTTGTTGCGATCATGGTTATAATG
TAAAAATAATTCTGGTATTAATTACATGTGGAAAGTGTTCTGCTTATAG
CTTTCTGCCTAAAATGCACGCTGCACGGGACAATATCATTGGTAATTTT
TTTAAAATCTGAATTGAGGCTACTCATAATACTATCCATAGGACATCAA
AGACATGTTGCATTGACTTTAAGCAGAGGTTCATCTAGAGGATTACTGC
ATAGGCTTGAACTACAAGTAATTTAAGGGACGAGAGCAACTTTAGCTCT
ACCACGTCGTTTTACAAGGTTATTAAAATCAAATTGATCTTATTAAAAC
TGAAAATTTGTAATAAAATGCTATTGAAAAATTAAAATATAGCAAACAC
CTAAATTGGACTGATTTTTAGATTCAAATTTAATAATTAATCTAAATTA
AACTTAAATTTTATAATATATGTCTTGTAATATATCAAGTTTTTTTTTT
TATTATTGAGTTTGGAAACATATAATAAGGAACATTAGTTAATATTGAT
AATCCACTAAGATCGACTTAGTATTACAGTATTTGGATGATTTGTATGA
GATATTCAAACTTCACTCTTATCATAATAGAGACAAAAGTTAATACTGA
TGGTGGAGAAAAAAAAATGTTATTGGGAGCATATGGTAAGATAAGACGG
ATAAAAATATGCTGCAGCCTGGAGAGCTAATGTATTTTTTGGTGAAGTT
TTCAAGTGACAACTATTCATGATGAGAACACAATAATATTTTCTACTTA
CCTATCCCACATAAAATACTGATTTTAATAATGATGATAAATAATGATT
AAAATATTTGATTCTTTGTTAAGAGAAATAAGGAAAACATAAATATTCT
CATGGAAAAATCAGCTTGTA
10) Seed specific GmFAD2B terminator sequence 1KB
(SEQ ID NO: 10)
TGAACCAAGCAATGGGCCATAGTGGGAGTTATGGAAGTTTTGTCACTTA
TCACTTAATTAGTAGAATGTTATAAATAAGTGGATTTGCCGCGTAATGA
CTTGTGTGCATTGTGAAACAGCTTGTAGCGATCCATGGCTATAATGTAA
AAATATGTGGAAAGTGTTCTGCTTATAACTTTCTTCCTTAAATGCACAC
TCCATAGAACAATATTATTGGTACTAATTTTAATCTGAATTGAGGATGT
TAACTGGTAGGTGAGGCCAACTTTAACCCTACTACTGTCGTTTTACAAG
GTTATTAAAATCGATTTGATGTTATTAAATTTGAAATAGTCGCCATTTA
AAAATAAAAAAATATAGAAGACAATTAAATTGGATTGGTTTTTAGATTC
AAATTTAAAAATTAATCTAAACTAAACTAAAATTTTATAATAACATGTC
TTATAATGTATTAAGTTTTTATTATTGTTATTGAGTTTGGAAGCATATA
ATAAGGAACATAAGTTAATATTTATGATCCAATAAGATCGACTTAGTAT
TAGAATATTTAGATGATTTGTATGAGATATTCAAACTTCATTCTTATCA
TAGAGAGACAAAAGTTAATATTGATTGTGGAGCAAAAAATGTTATTGGG
AGCATATGCAAAGATAAGATGGATAAAATATGCTGCAGCCTGCAGAGCA
AGTGTTTTGTCAAGTGACAACTATTCATGATGTGAACACAAGAATCCTT
TCTATTTATCTATCCCACATAAAATACTTATTTTAATAATGGTGGTAAA
TAATGATTAAAATATTTGATTATTGGTTAAAATAAATAAGGAAAATATA
AACATTCTCATGGAAAGTTTTTGTTAAAGAATTTGCCAAATATTGATTA
AAATATTTGATTCTTGGTTAGGAAAAATCAGCAATGCCTAAGAATATTT
TTTTCCAAACCTTGTAGAGTACCAAACACAATCTAAGAATTTTTCAAAT
CTATACGTGTTCAGAAACTT
11) GmFAD2A coding sequence
(SEQ ID NO: 11)
ATGGGTCTAGCAAAGGAAACAACAATGGGAGGTAGAGGTCGTGTGGCCA
AAGTGGAAGTTCAAGGGAAGAAGCCTCTCTCAAGGGTTCCAAACACAAA
GCCACCATTCACTGTTGGCCAACTCAAGAAAGCAATTCCACCACACTGC
TTTCAGCGCTCCCTCCTCACTTCATTCTCCTATGTTGTTTATGACCTTT
CATTTGCCTTCATTTTCTACATTGCCACCACCTACTTCCACCTCCTTCC
TCAACCCTTTTCCCTCATTGCATGGCCAATCTATTGGGTTCTCCAAGGT
TGCCTTCTCACTGGTGTGTGGGTGATTGCTCACGAGTGTGGTCACCATG
CCTTCAGCAAGTACCAATGGGTTGATGATGTTGTGGGTTTGACCCTTCA
CTCAACACTTTTAGTCCCTTATTTCTCATGGAAAATAAGCCATCGCCGC
CATCACTCCAACACAGGTTCCCTTGACCGTGATGAAGTGTTTGTCCCAA
AACCAAAATCCAAAGTTGCATGGTTTTCCAAGTACTTAAACAACCCTCT
AGGAAGGGCTGTTTCTCTTCTCGTCACACTCACAATAGGGTGGCCTATG
TATTTAGCCTTCAATGTCTCTGGTAGACCCTATGATAGTTTTGCAAGCC
ACTACCACCCTTATGCTCCCATATATTCTAACCGTGAGAGGCTTCTGAT
CTATGTCTCTGATGTTGCTTTGTTTTCTGTGACTTACTCTCTCTACCGT
GTTGCAACCCTGAAAGGGTTGGTTTGGCTGCTATGTGTTTATGGGGTGC
CTTTGCTCATTGTGAACGGTTTTCTTGTGACTATCACATATTTGCAGCA
CACACACTTTGCCTTGCCTCATTACGATTCATCAGAATGGGACTGGCTG
AAGGGAGCTTTGGCAACTATGGACAGAGATTATGGGATTCTGAACAAGG
TGTTTCATCACATAACTGATACTCATGTGGCTCACCATCTCTTCTCTAC
AATGCCACATTACCATGCAATGGAGGCAACCAATGCAATCAAGCCAATA
TTGGGTGAGTACTACCAATTTGATGACACACCATTTTACAAGGCACTGT
GGAGAGAAGCGAGAGAGTGCCTCTATGTGGAGCCAGATGAAGGAACATC
CGAGAAGGGCGTGTATTGGTACAGGAACAAGTATTGA
12) GmFAD2B coding sequence
(SEQ ID NO: 12)
ATGGGTCTAGCAAAGGAAACAATAATGGGAGGTGGAGGCCGTGTGGCCA
AAGTTGAAATTCAGCAGAAGAAGCCTCTCTCAAGGGTTCCAAACACAAA
GCCACCATTCACTGTTGGCCAACTCAAGAAAGCCATTCCACCGCACTGC
TTTCAGCGTTCCCTCCTCACTTCATTGTCCTATGTTGTTTATGACCTTT
CATTGGCTTTCATTTTCTACATTGCCACCACCTACTTCCACCTCCTCCC
TCACCCCTTTTCCCTCATTGCATGGCCAATCTATTGGGTTCTCCAAGGT
TGCATTCTTACTGGCGTGTGGGTGATTGCTCACGAGTGTGGTCACCATG
CCTTCAGCAAGTACCCATGGGTTGATGATGTTATGGGTTTGACCGTTCA
CTCAGCACTTTTAGTCCCTTATTTCTCATGGAAAATAAGCCATCGCCGC
CACCACTCCAACACGGGTTCCCTTGACCGTGATGAAGTGTTTGTCCCAA
AACCAAAATCCAAAGTTGCATGGTACACCAAGTACCTGAACAACCCTCT
AGGAAGGGCTGCTTCTCTTCTCATCACACTCACAATAGGGTGGCCTTTG
TATTTAGCCTTCAATGTCTCTGGCAGACCCTATGATGGTTTTGCTAGCC
ACTACCACCCTTATGCTCCCATATATTCAAATCGTGAGAGGCTTTTGAT
CTATGTCTCTGATGTTGCTTTGTTTTCTGTGACTTACTTGCTCTACCGT
GTTGCAACTATGAAAGGGTTGGTTTGGCTGCTATGTGTTTATGGGGTGC
CATTGCTCATTGTGAACGGTTTTCTTGTGACCATCACATATCTGCAGCA
CACACACTATGCCTTGCCTCACTATGATTCATCAGAATGGGATTGGCTG
AGGGGTGCTTTGGCAACTATGGACAGAGATTATGGAATTCTGAACAAGG
TGTTTCACCACATAACTGATACTCATGTGGCTCACCATCTTTTCTCTAC
AATGCCACATTACCATGCAACGGAGGCAACCAATGCAATGAAGCCAATA
TTGGGTGAGTACTACCGATTTGATGACACACCATTTTACAAGGCACTGT
GGAGAGAAGCAAGAGAGTGCCTCTATGTGGAGCCAGATGAAGGAACATC
CGAGAAGGGCGTGTATTGGTACAGGAACAAGTATTGA
13) Linear cis-genic cassette 1 [Nodule
Glymal3g44970 promoter - GmSACPD-C -
Glymal3g44970 terminator]
(SEQ ID NO: 13)
TTCTAACTTTATTATAATTCTATTATTGACTGACTTAAGCGTCAGAGTA
CCTTTACATGTACCACTCCCACCACCCGAAAAAGCTTAGAATACCAAGT
AGAAGATTAGATTATTGGTGGAGATCGATTTGAAGAGCACTTCAGTGGT
AAGAACATTTCCTAAATTACATTTCTAATTTGTCACAAAAATTTCATAC
TATAGATGCAAGTATTGTGCTAAAATGATGTAAGGCCCACGGAACTCAA
ATGTTTCCTTGGCTGTTGCAGATTCCCCGGCCCTAGAGACTTTGACGTT
TGATCCACACCACATGTCATCTCCTTTCAGAAGTATTGGACTCAGAGCA
GACTTAATATTATTGTGGCCACCTGAGCTAAATACTGGTGTGCTCTAAT
TAACTCTATGATGTGCTCTGCTTTCAGCAGCAGCCAATGCAGTCTGGCC
AAGTATGATGACTAAGCTCAAATCCTTTCCCAGTGCTACTAATTTCTTT
GTCCTCTGAACTAATACCAATAAGGATGGAATGTGCATATATATACATA
GATAGCAGGTCAACACAAACAATATAGCATATTACCATAATTCCATAAA
TTAATTGGACAATTTTCAATATCTCAATTCAATTGAAAACCTCTTGCAC
AAGGTGAACAGATACCATTGCGAAGCACATGTAATATAGTAAATGTCTA
ATGAACTAGAAGAATGCATTTATAGTTAAGTGTAGACATTAGACAGCAT
TAACAAATATATTGTCAGGGAGCAGAAGATCATTACCTTTCTGATTTCA
TCCAAGAGGTTATAAAACTGAGCATTGTGGGGACCATGTTGTTCATTAT
GGCAAAGGTCGTTCAGCATTTATCAAAAATCTGCTCATAAGGGAAGAAA
TCTCACTCGCGCGCGGTTAGGGCTCCATAGTCTTATCTCTATTTCTGCA
CCTGGTCCAATATTTGGCTTTTTCACCAAAAACAAGTTAAAAACGAAAA
CTAATTATTATAATATGTTGATCAAGAAAACATTTATTAAAATAAATTA
TTTTTGTTACTGTAGAAATATAAGAGGCACCTGGTCTAATATTCATTTT
GTGTTGGCTTGATGGATTATATTAAAATGATATATCCTGTTACAAAAAT
CATCTATTTTAATAAATATTTGTTTTTCCAACTCATTATACTTTTTTCG
TTTTTTAATGATGAAAAGGCTATCACTATTTATCCCTAAAAGTGATGGA
TTCGCAGGCCAGGACTGCACTTTAAAGTTCTAGAGAACTAATACTAGTA
CAGGTGCATAACATTAAAACATAAAGTGTTCATCATGTTAGAGAATACC
ACCCACTAGGGAGTCCTAAGGCCTTAGAAGGACTCAAGAGAAGGTGTGG
TAAAGAGTGTATCTTTTTATAATTAATTAAAAATAGTTATTAATTTTTT
ATTATATATTATGTACTTACGTATTTTTATTAAAAATTTAATATTTTTA
TAATTTATTATATATACTTACGTATTTTTGGTATAATTCTATTTATTAA
TTTTTAATTAACAATTTTTTTACTCTTTATCTATTAAAGTAATAAAGAA
TATAGAACATATGTGTGATAATCAAAACGTAGTAATTTTAATTTTTATT
TATAATTTTTAATTGACAATTATAACTTTATTTAACAATTATAACAAAT
TATTAAAAGAAGTTTTAAAGTATTATAATATTTGTTTATGTTGGAAAAT
GAATAAAATAAAATAAAAAAAAGGATGTGATAAAGGAAGATATATAATA
TTTAAAATTAACAAGCTTATCTCCATTACACATTTAAAATAATATATTT
GTAAAACAAGAGAAAGCACACTAAACCAGGGGCGAATAAATTTCTCTCC
CTTTGTTCCTGCTCCTGGTTGGCTTTACATTAGCATTTATAGCCAAGCC
AAGCTAGATCAAAGACAAAGTGTGTTGCTTAACGTTAACATGTTCCACT
AAAACAGAAAAATTAAGAGAGAAAGCTGAAAATTAATTTGATGCCTCCA
GAAAAGAAAGAAATTTTCAAGTCCTTGGAGGGATGGGCCTCGGAGTGGG
TCCTACCGCTGCTGAAGCCCGTGGAGCAATGCTGGCAGCCACAAAACTT
CCTCCCTGACCCCTCCCTTCCGCATGAAGAGTTCAGCCATCAGGTGAAG
GAGCTTCGCGAACGCACTAAAGAGTTACCTGATGAGTACTTTGTGGTGC
TGGTGGGTGATATGGTCACCGAGGACGCGCTTCCCACTTACCAGACCAT
GATCAACAACCTTGATGGAGTGAAAGATGACAGCGGCACGAGCCCGAGC
CCGTGGGCCGTGTGGACCCGGGCCTGGACCGCCGAGGAAAACAGACACG
GGGATCTGCTCAGAACTTATTTGTATCTCTCTGGGAGGGTTGACATGGC
TAAGGTCGAAAAGACCGTACATTACCTCATTTCAGCTGGCATGGACCCT
GGGACAGACAACAACCCATATTTGGGGTTTGTGTACACGTCATTCCAAG
AGCGAGCAACATTTGTGGCGCACGGGAACACGGCTCGGCTCGCGAAGGA
GGGCGGGGATCCAGTGCTGGCGCGCCTATGCGGGACCATCGCAGCGGAC
GAGAAGCGGCACGAGAACGCGTACTCAAGAATCGTGGAGAAGCTTCTGG
AAGTGGACCCCACCGGGGCAATGGTGGCCATAGGGAACATGATGGAGAA
GAAGATCACGATGCCGGCGCACCTTATGTACGATGGGGATGACCCCAGG
CTATTCGAGCACTACTCCGCTGTGGCGCAGCGCATAGGCGTGTACACCG
CCAACGACTACGCAGACATCTTGGAGTTTCTCGTTGAACGGTGGAGATT
GGAGAAGCTTGAAGGATTGATGGCTGAGGGGAAGCGGGCGCAGGATTTC
GTGTGTGGGTTGGCGCCGAGGATTAGGAGGTTGCAAGAGCGCGCTGATG
AGCGAGCGCGTAAGATGAAGAAGCATCATGGCGTTAAGTTCAGTTGGAT
TTTCAATAAAGAATTGCTTTTGTGACTAGCCTTTCAACCATCATTATAT
GCCGTACGTACGACTCAAATTAAATAAATAAACGGCTAGCTCAAGGTTT
CTATTTGCTTAATAAATTCTTGTCTTTTGTTATTATTCTCGTTGCACTC
GCACAGTTACTTCCCTGCTTATTGTTTTAGGAATACAATATAATTCTAT
ATATTTGAGTTTGAGTTGGTGCATACGACAAATCGACAATGCCAATTTC
TTTTTATACAATAAATTCTTGTATTTTTCTTTTATAATTACGATTAGGA
TGTTTTCTCACTAGAGAGCTTTGTATAATGTATCAGATTTGCGGATCTA
CTATTAGGATCGATGTTTACCCAATAAAGCATTATATCATGTATGCATA
AATAGTTTTGTAAAAAAAAATGTATATTGATCGTTGGTAATGTCACTAA
CTTTCGCTTGTAACTGTTGTTGGTAAATTGGTAATGTCGTCACTTACGA
ATTGGATCTATTAGGAGGTTAAATACGGGTCACCTTGTGACAGAGTTGA
TTGTTCTCAGATCCAGAACTACTAATTTGTTGGCTAACCTTAATATCTT
ATTTATTCATTCCAATCTTTTCCATTATCTATATATTCTCCAAGATCTT
TTTAGTTAAAGTTGTAAAATCAATTTTCCTGAAAAACAAAAAAAAAGGC
TAATAATACACTTTTTAATATATTATTTCTAACACATTTTATTATTAGT
TAAAATTTATTAATAACTATAAAATTAAGGGAAAAAACCATTAAATAAT
ATATTAAAAAGTATGTTTGAAATCCCAAACGTGTGAAAGTAGACTTGCT
AATGAGGTAAACTTGCTATTTACATTTTTCTTACGAGAGGAGATTCAAA
TAATAATTGTTTAGGGGTGTTGCATGTAATTGTAAAGGGGTTGTGTCTC
GGAAGGGTTTTTATTCTTTGTTGTCATGCAGGTTTGGACACTACTTGTC
TTAGAGCTGTATACCTTTCAAAAAATAAATAAATTCATTTTTTCT
14) Linear cis-genic cassette 2 [Seed FAD2A
promoter - GmFATB1A - FAD2A terminator]
(SEQ ID NO: 14)
TGCGTCAACATTTATATAATATATAGAAAAAAATTTGAAATTAATCACA
AAAACTAAAATTAAGAATTTGTCTAAAATAAGAATAAAGTATCTCAATT
AAAAAATAAAAACTAAAATCACAAATTTTAAAAAAGTGAAGGATAAAAT
GTATCATTTAAAAAATGGGAAAACGAAAATCACATATTTAAAAAAATAA
GAGATAGAAATTGCATTTTAATATTTTTTTTTATTTCTCTTCCTTTTTT
AATTATACTTTTAATCACATTAATGATTTTATTTTCTATTTCTCTTCTT
TCCACCTACATACATCCCAAAGATGGAGGGTGCAATTGTAAGTTTATTA
GCACTCTTGTTTTTACCTGCATTTGTGTGTGCTAACCAAATTGCATTCT
TCTCTTTACATAATGTATTTGATTTGAATTTTCATACCACATGCAAGCA
TGATTACGTACGTGTCCATGATCAAATACAAATGCTGTCTGGTACTGGC
AATTTGGTAAACAGCCATCCATTTTTTTTTGTCTCTAATTATTCTCTAG
AATATCTGAAGATTCCTCTGTCATCGAATTCCTTGCTTGGTAACAACGT
CGTCAAGTTATTATTTTGTTCTTTTTTTTTTTATCATATTTCTTATTTT
GTTCCAAGTATGTCATATTTTGATCCATCTTGACAAGTAGATTGTCATG
TAGGAATAGGAATATCACTTTAAATTTTAAAGCATTGATTAGTCTGTAG
GCAATATTGTCTTCTTCTTCCTCCTTATTAATATTTTTTATTCTGCCTT
CAATCACCAGTTATGGGAGATGGATGTAATACTAAATACCATAGTTGTT
CTGCTTGAAGTTTAGTTGTATAGTTGTTCTGCTTGAAGTTTAGTTGTGT
GTAATGTTTCAGCGTTGGCTTCCCCTGTAACTGCTACAATGGTACTGAA
TATATATTTTTTGCATTGTTCATTTTTTTCTTTTACTTAATCTTCATTG
CTTTGAAATTAATAAAACAAAAAGAAGGACCGAATAGTTTGAAGTTTGA
ACTATTGCCTATTCATGTAACTTATTCACCCAATCTTATATAGTTTTTC
TGGTAGAGATCATTTTAAATTGAAGGATATAAATTAAGAGGAAATACTT
GTATGTGATGTGTGGCAATTTGGAAGATCATGCGTAGAGAGTTTAATGG
CAGGTTTTGCAAATTGACCTGTAGTCATAATTACACTGGGCCCTCTCGG
AGTTTTGTGCCTTTTTGTTGTCGCTGTGTTTGGTTCTGCATGTTAGCCT
CACACAGATATTTAGTAGTTGTTGTTCTGCATATAAGCCTCACACGTAT
ACTAAACGAGTGAACCTCAAAATCATGGCCTTACACCTATTGAGTGAAA
TTAATGAACAGTGCATGTGAGTATGTGACTGTGACACAACCCCCGGTTT
TCATATTGCAATGTGCTACTGTGGTGATTAACCTTGCTACACTGTCGTC
CTTGTTTGTTTCCTTATGTATATTGATACCATAAATTATTACTAGTATA
TCATTTTATATTGTCCATACCATTACGTGTTTATAGTCTCTTTATGACA
TGTAATTGAATTTTTTAATTATAAAAAATAATAAAACTTAATTACGTAC
TATAAAGAGATGCTCTTGACTAGAATTGTGATCTCCTAGTTTCCTAACC
ATATACTAATATTTGCTTGTATTGATAGCCCCTCCGTTCCCAAGAGTAT
AAAACTGCATCGAATAATACAAGCCACTAGGCATGGTAAATTAAATTGT
GCCTGCACCTCGGGATATTTCATGTGGGGTTCATCATATTTGTTGAGGA
AAAGAAACTCCCGAAATTGAATTATGCATTTATATATCCTTTTTCATTT
CTAGATTTCCTGAAGGCTTAGGTGTAGGCACCTAGCTAGTAGCTACAAT
ATCAGCACTTCTCTCTATTGATAAACAATTGGCTGTAATGCCGCAGTAG
AGGACGATCACAACATTTCGTGCTGGTTACTTTTTGTTTTATGGTGGCA
ACAGCTGCTACTTCATCATTTTTCCCTGTTACTTCACCCTCGCCGGACT
CTGGTGGAGCAGGCAGCAAACTTGGTGGTGGGCCTGCAAACCTTGGAGG
ACTAAAATCCAAATCTGCGTCTTCTGGTGGCTTGAAGGCAAAGGCGCAA
GCCCCTTCGAAAATTAATGGAACCACAGTTGTTACATCTAAAGAAAGCT
TCAAGCATGATGATGATCTACCTTCGCCTCCCCCCAGAACTTTTATCAA
CCAGTTGCCTGATTGGAGCATGCTTCTTGCTGCTATCACAACAATTTTC
TTGGCCGCTGAAAAGCAGTGGATGATGCTTGATTGGAAGCCACGGCGAC
CTGACATGCTTATTGACCCCTTTGGGATAGGAAAAATTGTTCAGGATGG
TCTTGTGTTCCGTGAAAACTTTTCTATTAGATCATATGAGATTGGTGCT
GATCGTACCGCATCTATAGAAACAGTAATGAACCATTTGCAAGAAACTG
CACTTAATCATGTTAAAAGTGCTGGGCTTCTTGGTGATGGCTTTGGTTC
CACGCCAGAAATGTGCAAAAAGAACTTGATATGGGTGGTTACTCGGATG
CAGGTTGTGGTGGAACGCTATCCTACATGGGGTGACATAGTTCAAGTGG
ACACTTGGGTTTCTGGATCAGGGAAGAATGGTATGCGCCGTGATTGGCT
TTTACGTGACTGCAAAACTGGTGAAATCTTGACAAGAGCTTCCAGTGTT
TGGGTCATGATGAATAAGCTAACACGGAGGCTGTCTAAAATTCCAGAAG
AAGTCAGACAGGAGATAGGATCTTATTTTGTGGATTCTGATCCAATTCT
GGAAGAGGATAACAGAAAACTGACTAAACTTGACGACAACACAGCGGAT
TATATTCGTACCGGTTTAAGTCCTAGGTGGAGTGATCTAGATATCAATC
AGCATGTCAACAATGTGAAGTACATTGGCTGGATTCTGGAGAGTGCTCC
ACAGCCAATCTTGGAGAGTCATGAGCTTTCTTCCATGACTTTAGAGTAT
AGGAGAGAGTGTGGTAGGGACAGTGTGCTGGATTCCCTGACTGCTGTAT
CTGGGGCCGACATGGGCAATCTAGCTCACAGCGGGCATGTTGAGTGCAA
GCATTTGCTTCGACTGGAAAATGGTGCTGAGATTGTGAGGGGCAGGACT
GAGTGGAGGCCCAAACCTGTGAACAACTTTGGTGTTGTGAACCAGGTTC
CAGCAGAAAGCACCTAATGGAGCAACCAATGGGCCATAGTGGGAGTTAT
GGAAGTTTTGTCATGTATTAGTACATAATTAGTAGAATGTTATAAATAA
GTGGATTTGCCGCGTAATGACTTTGTGTGTATTGTGAAACAGCTTGTTG
CGATCATGGTTATAATGTAAAAATAATTCTGGTATTAATTACATGTGGA
AAGTGTTCTGCTTATAGCTTTCTGCCTAAAATGCACGCTGCACGGGACA
ATATCATTGGTAATTTTTTTAAAATCTGAATTGAGGCTACTCATAATAC
TATCCATAGGACATCAAAGACATGTTGCATTGACTTTAAGCAGAGGTTC
ATCTAGAGGATTACTGCATAGGCTTGAACTACAAGTAATTTAAGGGACG
AGAGCAACTTTAGCTCTACCACGTCGTTTTACAAGGTTATTAAAATCAA
ATTGATCTTATTAAAACTGAAAATTTGTAATAAAATGCTATTGAAAAAT
TAAAATATAGCAAACACCTAAATTGGACTGATTTTTAGATTCAAATTTA
ATAATTAATCTAAATTAAACTTAAATTTTATAATATATGTCTTGTAATA
TATCAAGTTTTTTTTTTTATTATTGAGTTTGGAAACATATAATAAGGAA
CATTAGTTAATATTGATAATCCACTAAGATCGACTTAGTATTACAGTAT
TTGGATGATTTGTATGAGATATTCAAACTTCACTCTTATCATAATAGAG
ACAAAAGTTAATACTGATGGTGGAGAAAAAAAAATGTTATTGGGAGCAT
ATGGTAAGATAAGACGGATAAAAATATGCTGCAGCCTGGAGAGCTAATG
TATTTTTTGGTGAAGTTTTCAAGTGACAACTATTCATGATGAGAACACA
ATAATATTTTCTACTTACCTATCCCACATAAAATACTGATTTTAATAAT
GATGATAAATAATGATTAAAATATTTGATTCTTTGTTAAGAGAAATAAG
GAAAACATAAATATTCTCATGGAAAAATCAGCTTGTA
15) TALEN GmSACPD-C-T01 targeting GmSACPD-C gene
coding sequence (hitSeq)
(SEQ ID NO: 15)
TGGAGGGATGGGCCTCGGAGTGGGTCCTACCGCTGCTGAAGCCCGTGGA
16) TALEN GmSACPD-C-T01 targeting GmSACPD-C gene
coding sequence (leftSeq)
(SEQ ID NO: 16)
TGGAGGGATGGGCCTCG
17) TALEN GmSACPD-C-T01 targeting GmSACPD-C gene
coding sequence (rtSeq)
(SEQ ID NO: 17)
TCCACGGGCTTCAGCAG
18) TALEN GmSACPD-C-T02 targeting GmSACPD-C gene
coding sequence (hitSeq)
(SEQ ID NO: 18)
TGGGTCCTACCGCTGCTGAAGCCCGTGGAGCAATGCTGGCAGCCACAAA
19) TALEN GmSACPD-C-T02 targeting GmSACPD-C gene
coding sequence (leftSeq)
(SEQ ID NO: 19)
TGGGTCCTACCGCTGCT
20) TALEN GmSACPD-C-T02 targeting GmSACPD-C gene
coding sequence (rtSeq)
(SEQ ID NO: 20)
TTTGTGGCTGCCAGCAT
21) TALEN GmSACPD-C-T03 targeting GmSACPD-C gene
coding sequence (hitSeq)
(SEQ ID NO: 21)
TCCTCCCTGACCCCTCCCTTCCGCATGAAGAGTTCAGCCATCAGGTGAA
22) TALEN GmSACPD-G-T03 targeting GmSACPD-C gene
coding sequence (leftSeq)
(SEQ ID NO: 22)
TCCTCCCTGACCCCTCC
23) TALEN GmSACPD-C-T03 targeting GmSACPD-C gene
coding sequence (rtSeq)
(SEQ ID NO: 23)
TTCACCTGATGGCTGAA
24) TALEN GmSACPD-C-T04 targeting GmSACPD-C gene
coding sequence (hitSeq)
(SEQ ID NO: 24)
TCCCTGACCCCTCCCTTCCGCATGAAGAGTTCAGCCATCAGGTGAAGGA
25) TALEN GmSACPD-C-T04 targeting GmSACPD-C gene
coding sequence (leftSeq)
(SEQ ID NO: 25)
TCCCTGACCCCTCCCTT
26) TALEN GmSACPD-C-T04 targeting GmSACPD-C gene
coding sequence (rtSeq)
(SEQ ID NO: 26)
TCCTTCACCTGATGGCT
27) TALEN GmSACPD-C-T05 targeting GmSACPD-C gene
coding sequence (hitSeq)
(SEQ ID NO: 27)
TACCTGATGAGTACTTTGTGGTGCTGGTGGGTGATATGGTCACCGAGGA
28) TALEN GmSACPD-C-T05 targeting GmSACPD-C gene
coding sequence (leftSeq)
(SEQ ID NO: 28)
TACCTGATGAGTACTTT
29) TALEN GmSACPD-C-T05 targeting GmSACPD-C gene
coding sequence (rtSeq)
(SEQ ID NO: 29)
TCCTCGGTGACCATATC
30) TALEN GmSACPD-C-T06 targeting GmSACPD-C gene
5′UTR (hitSeq)
(SEQ ID NO: 30)
TCCGCGGCGCCGTTCAAAGCCCGGAAGGCCCACTCAATGCCTCCAGAAA
31) TALEN GmSACPD-C-T06 targeting GmSACPD-C gene
5′UTR (leftSeq)
(SEQ ID NO: 31)
TCCGCGGCGCCGTTCAA
32) TALEN GmSACPD-C-T06 targeting GmSACPD-C gene
5′UTR (rtSeq)
(SEQ ID NO: 32)
TTTCTGGAGGCATTGAG
33) TALEN GmSACPD-C-T07 targeting GmSACPD-C gene
5′UTR (hitSeq)
(SEQ ID NO: 33)
TAAATTATCAACAAACCAAGGGCTAATCACTAGTCACACCCTTTACAAA
34) TALEN GmSACPD-C-T07 targeting GmSACPD-C gene
5′UTR (leftSeq)
(SEQ ID NO: 34)
TAAATTATCAACAAACC
35) TALEN GmSACPD-C-T07 targeting GmSACPD-C gene
5′UTR (rtSeq)
(SEQ ID NO: 35)
TTTGTAAAGGGTGTGAC
36) TALEN GmSACPD-C-TO8 targeting GmSACPD-C gene
5′UTR (hitSeq)
(SEQ ID NO: 36)
TAGTCACACCCTTTACAAATATCTCCAACCTCTCCACAGTTCCACTCAA
37) TALEN GmSACPD-C-T08 targeting GmSACPD-C gene
5′UTR (leftSeg)
(SEQ ID NO: 37)
TAGTCACACCCTTTACA
38) TALEN GmSACPD-C-T08 targeting GmSACPD-C gene
5′UTR (rtSeq)
(SEQ ID NO: 38)
TTGAGTGGAACTGTGGA
39) TALEN GmSACPD-C-T09 targeting GmSACPD-C gene
5′UTR (hitSeq)
(SEQ ID NO: 39)
TCAAGTACAATAGACACGTAATCAAAACCATGCAGATACGAACCTGCCA
40) TALEN GmSACPD-C-T09 targeting GmSACPD-C gene
5′UTR (leftSeg)
(SEQ ID NO: 40)
TCAAGTACAATAGACAC
41) TALEN GmSACPD-C-T09 targeting GmSACPD-C gene
5′UTR (rtSeq)
(SEQ ID NO: 41)
TGGCAGGTTCGTATCTG
42) TALEN GmSACPD-C-T10 targeting GmSACPD-C gene
5′UTR (hitSeq)
(SEQ ID NO: 42)
TCACCACCCAAACCCTTCCACAACTTCCGTGTTCTTCTAGAAAAGCCCA
43) TALEN GmSACPD-C-T10 targeting GmSACPD-C gene
5′UTR (leftSeg)
(SEQ ID NO: 43)
TCACCACCCAAACCCTT
44) TALEN GmSACPD-C-T10 targeting GmSACPD-C gene
5′UTR (rtSeq)
(SEQ ID NO: 44)
TGGGCTTTTCTAGAAGA
45) TALEN GmSACPD-C-T11 targeting GmSACPD-C gene
5′UTR (hitSeq)
(SEQ ID NO: 45)
TTCCGCCGTTAAACGCTGCGGTTTCCGCGGCGCCGTTCAAAGCCCGGAA
46) TALEN GmSACPD-C-T11 targeting GmSACPD-C gene
5′UTR (leftSeq)
(SEQ ID NO: 46)
TTCCGCCGTTAAACGCT
47) TALEN GmSACPD-C-T11 targeting GmSACPD-C gene
5′UTR (rtSeq)
(SEQ ID NO: 47)
TTCCGGGCTTTGAACGG
48) TALEN GmFATB1A-T01 targeting GmFATB-1A gene
5′UTR (hitSeq)
(SEQ ID NO: 48)
TTAGTCCGATTGATTTCTCGATATCATTTAAGGCTAAGGTTGACCTCTA
49) TALEN GmFATB1A-T01 targeting GmFATB-1A gene
5′UTR (leftSeq)
(SEQ ID NO: 49)
TTAGTCCGATTGATTTC
50) TALEN GmFATB1A-T01 targeting GmFATB-1A gene
5′UTR (rtSeq)
(SEQ ID NO: 50)
TAGAGGTCAACCTTAGC
51) TALEN GmFATB1A-T02 targeting GmFATB-1A gene
5′UTR (hitSeq)
(SEQ ID NO: 51)
TCTTCTAACTTGCGTATATTTTGCATGCAGCGACCTTAGAAATTCATTA
52) TALEN GmFATB1A-T02 targeting GmFATB-1A gene
5′UTR (leftSeq)
(SEQ ID NO: 52)
TCTTCTAACTTGCGTAT
53) TALEN GmFATB1A-T02 targeting GmFATB-1A gene
5′UTR (rtSeq)
(SEQ ID NO: 53)
TAATGAATTTCTAAGGT
54) TALEN GmFATB1A-T03 targeting GmFATB-1A gene
5′UTR (hitSeq)
(SEQ ID NO: 54)
TTTGCATTTCTCTTCTTTATCCCCTTTCTGTGGAAGGTGGGAGGGAAAA
55) TALEN GmFATB1A-T03 targeting GmFATB-1A gene
5′UTR (leftSeq)
(SEQ ID NO: 55)
TTTGCATTTCTCTTCTT
56) TALEN GmFATB1A-T03 targeting GmFATB-1A gene
5′UTR (rtSeq)
(SEQ ID NO: 56)
TTTTCCCTCCCACCTTC
57) TALEN GmFATB1A-T04 targeting GmFATB-1A gene
5′UTR (hitSeq)
(SEQ ID NO: 57)
TGTGATATAACTGATGTGCTGTGCTGTTATTATTTGTTATTTGGGGTGA
58) TALEN GmFATB1A-T04 targeting GmFATB-1A gene
5′UTR (leftSeq)
(SEQ ID NO: 58)
TGTGATATAACTGATGT
59) TALEN GmFATB1A-T04 targeting GmFATB-1A gene
5′UTR (rtSeq)
(SEQ ID NO: 59)
TCACCCCAAATAACAAA
60) TALEN GmFATB1A-T05 targeting GmFATB-1A gene
5′UTR (hitSeq)
(SEQ ID NO: 60)
TTATTATTTGTTATTTGGGGTGAAGTATAATTTTTTGGGTGAACTTGGA
61) TALEN GmFATB1A-T05 targeting GmFATB-1A gene
5′UTR (leftSeg)
(SEQ ID NO: 61)
TTATTATTTGTTATTTG
62) TALEN GmFATB1A-T05 targeting GmFATB-1A gene
5′UTR (rtSeq)
(SEQ ID NO: 62)
TCCAAGTTCACCCAAAA
63) GmSACPD-C fragment Bert wt
(SEQ ID NO: 63)
CAATGCCTCCAGAAAAGAAAGAAATTTTCAAGTCCTGGAGGGATGGGCC
TCGGAGTGGGTCCTACCGCTGCTGAAGCCCGTGGAGCAATGCTGGCAGC
CACAAAACTTCCTCCCTGACCCCTCCCTTCCGCATGAAGAGTTCAGCCA
TCAGGTGAAGGAGCTTCGCGAACGCACTAAAGAGTTACCTGATGAGTAC
TTTGTGGTGCTGGTGGGTGATATGGTCACCGAGGACGCGCTTCCCACTT
ACCAGACCATGATCAACAACCTTGATGGAGTGAAAGATGACAGCGGCAC
GAG
64) GmSACPD-C fragment T₀ event #l(-26 nt)
(SEQ ID NO: 64)
CAATGCCTCCAGAAAAGAAAGAAATTTTCAAGTCCTTGGAGGGATGGGC
CTCGGAGTGGGTCCTACCGCTGCTGAAGCCCGTGGAGCAATGCTGGCAG
CCACAAAACTTCCTCCCTCAGCCATCAGGTGAAGGAGCTTCGCGAACGC
ACTAAAGAGTTACCTGATGAGTACTTTGTGGTGCTGGTGGGTGATATGG
TCACCGAGGACGCGCTTCCCACTTACCAGACCATGATCAACAACCTTGA
TGGAGTGAAAGATGACAGCGGCACGAG
65) GmSACPD-C fragment T₀ event #2 (-14 nt)
(SEQ ID NO: 65)
CAATGCCTCCAGAAAAGAAAGAAATTTTCAAGTCCTTGGAGGGATGGGC
CTCGGAGTGGGTCCTACCGCTGCTGAAGCCCGTGGAGCAATGCTGGCAG
CCACAAAACTTCCTCCCTGACCCCTCCCTTCCGCCATCAGGTGAAGGAG
CTTCGCGAACGCACTAAAGAGTTACCTGATGAGTACTTTGTGGTGCTGG
TGGGTGATATGGTCACCGAGGACGCGCTTCCCACTTACCAGACCATGAT
CAACAACCTTGATGGAGTGAAAGATGACAGCGGCACGAG
66) GmSACPD-C fragment T₀ event #3 (-52 nt)
(SEQ ID NO: 66)
CAATGCCTCCAGAAAAGAAAGAAATTTTCAAGTCCTTGGAGGGATGGGC
CTCGGAGTGGGTCCTACCGCTGCTGAAGCCCGTGAAGAGTTCAGCCATC
AGGTGAAGGAGCTTCGCGAACGCACTAAAGAGTTACCTGATGAGTACTT
TGTGGTGCTGGTGGGTGATATGGTCACCGAGGACGCGCTTCCCACTTAC
CAGACCATGATCAACAACCTTGATGGAGTGAAAGATGACAGCGGCACGA
G
67) GmSACPD-C fragment T₀ event #4 Allele 1
(-5 nt)
(SEQ ID NO: 67)
CAATGCCTCCAGAAAAGAAAGAAATTTTCAAGTCCTTGGAGGGATGGGC
CTCGGAGTGGGTCCTACCGCTGCTGAAGCCCGTGGAGCAATGCTGGCAG
CCACAAAACTTCCTCCCTGACCCCTCCCTTCGAAGAGTTCAGCCATCAG
GTGAAGGAGCTTCGCGAACGCACTAAAGAGTTACCTGATGAGTACTTTG
TGGTGCTGGTGGGTGATATGGTCACCGAGGACGCGCTTCCCACTTACCA
GACCATGATCAACAACCTTGATGGAGTGAAAGATGACAGCGGCACGAG
68) GmSACPD-C fragment T₀ event #4 Allele 2
(-7/+3 nt)
(SEQ ID NO: 68)
CAATGCCTCCAGAAAAGAAAGAAATTTTCAAGTCCTTGGAGGGATGGGC
CTCGGAGTGGGTCCTACCGCTGCTGAAGCCCGTGGAGCAATGCTGGCAG
CCACAAAACTTCCTCCCTGACCCCTCCCTTCGTCAGAGTTCAGCCATCA
GGTGAAGGAGCTTCGCGAACGCACTAAAGAGTTACCTGATGAGTACTTT
GTGGTGCTGGTGGGTGATATGGTCACCGAGGACGCGCTTCCCACTTACC
AGACCATGATCAACAACCTTGATGGAGTGAAAGATGACAGCGGCACGAG
69) GmSACPD-C fragment T₀ event #5 Allele 1
(-8 nt)
(SEQ ID NO: 69)
CAATGCCTCCAGAAAAGAAAGAAATTTTCAAGTCCTTGGAGGGATGGGC
CTCGGAGTGGGTCCTACCGCTGCTGAAGCCCGTGGAGCAATGCTGGCAG
CCACAAAACTTCCTCCCTGACCCCTCCCTAAGAGTTCAGCCATCAGGTG
AAGGAGCTTCGCGAACGCACTAAAGAGTTACCTGATGAGTACTTTGTGG
TGCTGGTGGGTGATATGGTCACCGAGGACGCGCTTCCCACTTACCAGAC
CATGATCAACAACCTTGATGGAGTGAAAGATGACAGCGGCACGAG
70) GmSACPD-C fragment T₀ event #5 Allele 2
(-14 nt)
(SEQ ID NO: 70)
CAATGCCTCCAGAAAAGAAAGAAATTTTCAAGTCCTTGGAGGGATGGGC
CTCGGAGTGGGTCCTACCGCTGCTGAAGCCCGTGGAGCAATGCTGGCAG
CCACAAAACTTCCTCCCTGACCCCTCCCTTCAGCCATCAGGTGAAGGAG
CTTCGCGAACGCACTAAAGAGTTACCTGATGAGTACTTTGTGGTGCTGG
TGGGTGATATGGTCACCGAGGACGCGCTTCCCACTTACCAGACCATGAT
CAACAACCTTGATGGAGTGAAAGATGACAGCGGCACGAG
71) GmSACPD-C fragment Bert wt
(SEQ ID NO: 71)
ACCGCTGCTGAAGCCCGTGGAGCAATGCTGGCAGCCACAAAACTTCCTC
CCTGACCCCTCCCTTCCGCATGAAGAGTTCAGCCATCAGGTGAAGGAGC
TT
72) GmSACPD-C fragment T₀ event #1
(SEQ ID NO: 72)
ACCGCTGCTGAAGCCCGTGGAGCAATGCTGGCAGCCACAAAACTTCCTC
CCTCAGCCATCAGGTGAAGGAGCTT
73) GmSACPD-C fragment T₀ event #2
(SEQ ID NO: 73)
ACCGCTGCTGAAGCCCGTGGAGCAATGCTGGCAGCCACAAAACTTCCTC
CCTGACCCCTCCCTTCCGCCATCAGGTGAAGGAGCTT
74) GmSACPD-C fragment T₀ event #3
(SEQ ID NO: 74)
ACCGCTGCTGAAGCCCGTGAAGAGTTCAGCCATCAGGTGAAGGAGCTT
75) GmSACPD-C fragment T₀ event #4 Allele 1
(SEQ ID NO: 75)
ACCGCTGCTGAAGCCCGTGGAGCAATGCTGGCAGCCACAAAACTTCCTC
CCTGACCCCTCCCTTCGAAGAGTTCAGCCATCAGGTGAAGGAGCTT
76) GmSACPD-C fragment T₀ event #4 Allele 2
(SEQ ID NO: 76)
ACCGCTGCTGAAGCCCGTGGAGCAATGCTGGCAGCCACAAAACTTCCTC
CCTGACCCCTCCCTTCGTCAGAGTTCAGCCATCAGGTGAAGGAGCTT
77) GmSACPD-C fragment T₀ event #5 Allele 1
(SEQ ID NO: 77)
ACCGCTGCTGAAGCCCGTGGAGCAATGCTGGCAGCCACAAAACTTCCTC
CCTGACCCCTCCCTAAGAGTTCAGCCATCAGGTGAAGGAGCTT
78) GmSACPD-C fragment T₀ event #5 Allele 2
(SEQ ID NO: 78)
ACCGCTGCTGAAGCCCGTGGAGCAATGCTGGCAGCCACAAAACTTCCTC
CCTGACCCCTCCCTTCAGCCATCAGGTGAAGGAGCTT
79) Artificial sequence for seed-specific
silencing of SACPD-C (targeted promoter
replacement donor template sequence LHA
(SACPD-C) - Nodule Pro & 5′UTR (Glymal3g44970) -
RHA (SACPD-C))
(SEQ ID NO: 79)
ATACGTATATAATATTTAATTATATATACTTTGATCAATATGTTTAGTG
ATATTTAAAAATAATTCTACAAATAATTAATTAGCAAGTATTTATAAAA
TCCCTCAAAGAATCTATACCCAAAAACAGCTAGCAAAAATAATCATGCA
TCGAGTTTTAAAAACATAATAACTCTAATAAATAATTAATCAAATAAAT
TTAATTTCCTAGTCGAGCTTGTAATTTGTATGGTTAAATCATATTCAAA
TAGCAATGATTTTATATATTTTCATATTAAATTTTCATTTTTATGTGAA
GTGAAAATTTAAATTTAAGATATATGTACGAATAAAGCTAACAAATAAA
ATATTAAATTATACAAGTTAATTTCTTTTACAGTAAATTATATTCAATT
CAATCATCTTTTTCATCATATGATTGAAGATATATATTTATCTTAAGTA
AATACTATTTTATTTCCATATCTTTTGTTTCATACTTTAAAATAGAAGC
TTCCTTATTGTTATTATTTCTTGTAAGTTATTAGACCCAAAATCTTTCA
TATACACAAAATTATCTTTAAATTAATATAAAAATATTAATAACATATA
TTTCATAAAATATCAAAATTTATATCCCTGAAAAAAATTGTAGTGATGT
TTTCTTTTAGAGAAAAAATGATTATGAACACTGCACTATCATATCATAA
TCCACTGTTAACTTTTAAAATTATCTTAAAATAATTTTGTTTATAAATG
ACAATATAAAATTATTTCTAACTTTATTATAATTCTATTATTGACTGAC
TTAAGCGTCAGAGTACCTTTACATGTACCACTCCCACCACCCGAAAAAG
CTTAGAATACCAAGTAGAAGATTAGATTATTGGTGGAGATCGATTTGAA
GAGCACTTCAGTGGTAAGAACATTTCCTAAATTACATTTCTAATTTGTC
ACAAAAATTTCATACTATAGATGCAAGTATTGTGCTAAAATGATGTAAG
GCCCACGGAACTCAAATGTTTCCTTGGCTGTTGCAGATTCCCCGGCCCT
AGAGACTTTGACGTTTGATCCACACCACATGTCATCTCCTTTCAGAAGT
ATTGGACTCAGAGCAGACTTAATATTATTGTGGCCACCTGAGCTAAATA
CTGGTGTGCTCTAATTAACTCTATGATGTGCTCTGCTTTCAGCAGCAGC
CAATGCAGTCTGGCCAAGTATGATGACTAAGCTCAAATCCTTTCCCAGT
GCTACTAATTTCTTTGTCCTCTGAACTAATACCAATAAGGATGGAATGT
GCATATATATACATAGATAGCAGGTCAACACAAACAATATAGCATATTA
CCATAATTCCATAAATTAATTGGACAATTTTCAATATCTCAATTCAATT
GAAAACCTCTTGCACAAGGTGAACAGATACCATTGCGAAGCACATGTAA
TATAGTAAATGTCTAATGAACTAGAAGAATGCATTTATAGTTAAGTGTA
GACATTAGACAGCATTAACAAATATATTGTCAGGGAGCAGAAGATCATT
ACCTTTCTGATTTCATCCAAGAGGTTATAAAACTGAGCATTGTGGGGAC
CATGTTGTTCATTATGGCAAAGGTCGTTCAGCATTTATCAAAAATCTGC
TCATAAGGGAAGAAATCTCACTCGCGCGCGGTTAGGGCTCCATAGTCTT
ATCTCTATTTCTGCACCTGGTCCAATATTTGGCTTTTTCACCAAAAACA
AGTTAAAAACGAAAACTAATTATTATAATATGTTGATCAAGAAAACATT
TATTAAAATAAATTATTTTTGTTACTGTAGAAATATAAGAGGCACCTGG
TCTAATATTCATTTTGTGTTGGCTTGATGGATTATATTAAAATGATATA
TCCTGTTACAAAAATCATCTATTTTAATAAATATTTGTTTTTCCAACTC
ATTATACTTTTTTCGTTTTTTAATGATGAAAAGGCTATCACTATTTATC
CCTAAAAGTGATGGATTCGCAGGCCAGGACTGCACTTTAAAGTTCTAGA
GAACTAATACTAGTACAGGTGCATAACATTAAAACATAAAGTGTTCATC
ATGTTAGAGAATACCACCCACTAGGGAGTCCTAAGGCCTTAGAAGGACT
CAAGAGAAGGTGTGGTAAAGAGTGTATCTTTTTATAATTAATTAAAAAT
AGTTATTAATTTTTTATTATATATTATGTACTTACGTATTTTTATTAAA
AATTTAATATTTTTATAATTTATTATATATACTTACGTATTTTTGGTAT
AATTCTATTTATTAATTTTTAATTAACAATTTTTTTACTCTTTATCTAT
TAAAGTAATAAAGAATATAGAACATATGTGTGATAATCAAAACGTAGTA
ATTTTAATTTTTATTTATAATTTTTAATTGAGAATTATAACTTTATTTA
ACAATTATAACAAATTATTAAAAGAAGTTTTAAAGTATTATAATATTTG
TTTATGTTGGAAAATGAATAAAATAAAATAAAAAAAAGGATGTGATAAA
GGAAGATATATAATATTTAAAATTAACAAGCTTATCTCCATTACACATT
TAAAATAATATATTTGTAAAACAAGAGAAAGCACACTAAACCAGGGGCG
AATAAATTTCTCTCCCTTTGTTCCTGCTCCTGGTTGGCTTTACATTAGC
ATTTATAGCCAAGCCAAGCTAGATCAAAGACAAAGTGTGTTGCTTAACG
TTAACATGTTCCACTAAAACAGAAAAATTAAGAGAGAAAGCTGAAAATT
AATTTGATGCCTCCAGAAAAGAAAGAAATTTTCAAGTCCTTGGAGGGAT
GGGCCTCGGAGTGGGTCCTACCGCTGCTGAAGCCCGTGGAGCAATGCTG
GCAGCCACAAAACTTCCTCCCTGACCCCTCCCTTCCGCATGAAGAGTTC
AGCCATCAGGTGAAGGAGCTTCGCGAACGCACTAAAGAGTTACCTGATG
AGTACTTTGTGGTGCTGGTGGGTGATATGGTCACCGAGGACGCGCTTCC
CACTTACCAGACCATGATCAACAACCTTGATGGAGTGAAAGATGACAGC
GGCACGAGCCCGAGCCCGTGGGCCGTGTGGACCCGGGCCTGGACCGCCG
AGGAAAACAGACACGGGGATCTGCTCAGAACTTATTTGTATCTCTCTGG
GAGGGTTGACATGGCTAAGGTCGAAAAGACCGTACATTACCTCATTTCA
GCTGGCATGGTAAGTGTTTCACTGTTTTTTATTTTTTATTTATAATTTG
AATTCGGTTGACTAGATTCTATTGATTGGGTGAGTATGCATTTCTTTTT
TACTTGACATATGGCAGGAGTAATTTCTTTTGTATATACGTGTTTAAAA
TTGGATATTGTTCGTCTAGCGATTTTTGCTTCTCTTCATTTTAATTTCT
GAAGGTTTCGAGATGTACCATTCCACATGAAGCAAAAATTAATTGAAAT
TTATAATTTAAAAGAGGTGATATTCTATGTACTATTAAATTGAACAAAA
TTGTTATTCATAACAATATAT
80) Expected edited RHA sequence - Nodule
promoter/UTR insert - RHA
(SEQ ID NO: 80)
CATTAGACATTTAAAATAATATATTTGTAAAACAAGAGAAAGCACACTA
AACCAGGGGCGAATAAATTTCTCTCCCTTTGTTCCTGCTCCTGGTTGGC
TTTACATTAGCATTTATAGCCAAGCCAAGCTAGATCAAAGACAAAGTGT
GTTGCTTAACGTTAACATGTTCCACTAAAACAGAAAAATTAAGAGAGAA
AGCTGAAAATTAATTTGATGCCTCCAGAAAAGAAAGAAATTTTCAAGTC
CTTGGAGGGATGGGCCTCGGAGTGGGTCCTACCGCTGCTGAAGCCCGTG
GAGCAATGCTGGCAGCCACAAAACTTCCTCCCTGACCCCTCCCTTCCGC
ATGAAGAGTTCAGCCATCAGGTGAAGGAGCTTCGCGAACGCACTAAAGA
GTTACCTGATGAGTACTTTGTGGTGCTGGTGGGTGATATGGTCACCGAG
GACGCGCTTCCCACTTACCAGACCATGATCAACAACCTTGATGGAGTGA
AAGATGACAGCGGCACGAGCCCGAGCCCGTGGGCCGTGTGGACCCGGGC
CTGGACCGCCGAGGAAAACAGACACGGGGATCTGCTCAGAACTTATTTG
TATCTCTCTGGGAGGGTTGACATGGCTAAGGTCGAAAAGACCGTACATT
ACCTCATTTCAGCTGGCATGGTAAGTGTTTCACTGTTTTTTATTTTTTA
TTTATAATTTGAATTCGGTTGACTAGATTCTATTGATTGGGTGAGTATG
CATTTCTTTTTTACTTGACATATGGCAGGAGTAATTTCTTTTGTATATA
CGTGTTTAAAATTGGATATTGTTCGTCTAGCGATTTTTGCTTCTCTTCA
TTTTAATTTCTGAAGGTTTCGAGATGTACCATTCCACATGAAGCAAAAA
TTAATTGAAATTTATAATTTAAAAGAGGTGATATTCTATGTACTATTAA
ATTGAACAAAATTGTTATTCATAACAATATATAAAAGAAAATATTTCCT
TTTTTGATCTCTACATTTGTCGATTATTTTGTCTTTTAATTGTTTTTAA
AACTACTCACTACTATATTCTCTTCCATTTTTTATTTAACATCTACTCT
CTATTCTTCTTTCATTTTTTTTTGTTTTATAAAAAGTACAGAGACTCTT
CAATTTTCTATTTAACGTTTACTCTTTATTTCTCTTTCTTTTTATATTT
TAACAAGAAAAATAGAGTTCTGTATCTCTCTTTAATTTATATATAAGCT
CTGTAGCATGCCGGTCCACATCACATATAATAGTTTAAAATTAAAATTA
ATTAATGTACTTAACAGCTGCATAGCTCATATTGGTATATTAAATTGTA
CAAGAAAAACTGAAAAGCCAAACAAACATTTTTAGTATCAATAAATTCT
TGGTTTCACGTGACATGGCAATCGGAGCTTTCTCATAGCAAGTTGGGCA
GACTTAAATTGAACAGTTAAGTAGATTT
81) Artificial sequence for seed-specific
upregulation of FATB-1A (targeted promoter
replacement donor template sequence LHA
(FATB-1A) - FAD2A Pro & 5′ UTR (Glyma10g42470) -
RHA (FATB-1A))
CTGTTGTTACTTTTCATACTATATTTATATCAACTATTTGCTTAACAAC
AGGAAACTGCACTTAATCATGTTAAAAGTGCTGGGCTTCTTGGTGATGG
CTTTGGTTCCACGCCAGAAATGTGCAAAAAGAACTTGATATCTATGAAA
ATATTCGAAATTCATTCATGTGAAATTTTTAGTATATTTTTTATTTACA
TAAAATTAAAATTTATTATTTTTTACCTAGATATAATCTTGAGTAAATT
TAAACTTATGGTGATGATTTTATATCTATGAAAAATGTGATTTTTTTTA
TATTGGTGGTTACAACCAATATAAAAAATAATACAAATAAGTTCAAATA
TTTAAATCTTACTGGGGTTAAAAATTGACACGTTTCAATTTTATAAACA
TGAAAAATATTCTTGAAAATTTTAAAAAACAAATTTTGAATATTTTTAT
ATTTATGATAATTAAAAATATATTTTAATCTAATAAAAATGCATTAAAA
AAGAATAGTAGCTAATATATAAAATTAAAATTCTAAAGTAGAAAAAAAA
GATTAAACCTTATTTTTATCGAGTAAACTTGATAGAGATAAATAATGAT
ACGTGGTGGTGGGGTGATTACAAAATGTCTTAATCTTTTATTGTGAAAG
AAATATTCTATTGTGAATAAAAAAAAAAACCCAATGTCTTTATCTTTTA
CTTGGAAAATGAAAAAAAAAAAAAAAACTCTGTAAAATCCCGTGGGTAC
TGGCATTACTAGGAGAGTATGGCCGAAATAAGGGGGGCAATACGGTAAA
AGAGGGAAGAGACTAGCTGGGATCTTTGAAAGGGCGGCGGGAGGGCCAG
CTGGACAGTATAAAAAGAAGTGGCTGGAATGCTAATGCCTTATCCCTAA
CTCATAATGCGTCAACATTTATATAATATATAGAAAAAAATTTGAAATT
AATCACAAAAACTAAAATTAAGAATTTGTCTAAAATAAGAATAAAGTAT
CTCAATTAAAAAATAAAAACTAAAATCACAAATTTTAAAAAAGTGAAGG
ATAAAATGTATCATTTAAAAAATGGGAAAACGAAAATCACATATTTAAA
AAAATAAGAGATAGAAATTGCATTTTAATATTTTTTTTTATTTCTCTTC
CTTTTTTAATTATACTTTTAATCACATTAATGATTTTATTTTCTATTTC
TCTTCTTTCCACCTACATACATCCCAAAGATGGAGGGTGCAATTGTAAG
TTTATTAGCACTCTTGTTTTTACCTGCATTTGTGTGTGCTAACCAAATT
GCATTCTTCTCTTTACATAATGTATTTGATTTGAATTTTCATACCACAT
GCAAGCATGATTACGTACGTGTCCATGATCAAATACAAATGCTGTCTGG
TACTGGCAATTTGGTAAACAGCCATCCATTTTTTTTTGTCTCTAATTAT
TCTCTAGAATATCTGAAGATTCCTCTGTCATCGAATTCCTTGCTTGGTA
ACAACGTCGTCAAGTTATTATTTTGTTCTTTTTTTTTTTATCATATTTC
TTATTTTGTTCCAAGTATGTCATATTTTGATCCATCTTGACAAGTAGAT
TGTCATGTAGGAATAGGAATATCACTTTAAATTTTAAAGCATTGATTAG
TCTGTAGGCAATATTGTCTTCTTCTTCCTCCTTATTAATATTTTTTATT
CTGCCTTCAATCACCAGTTATGGGAGATGGATGTAATACTAAATACCAT
AGTTGTTCTGCTTGAAGTTTAGTTGTATAGTTGTTCTGCTTGAAGTTTA
GTTGTGTGTAATGTTTCAGCGTTGGCTTCCCCTGTAACTGCTACAATGG
TACTGAATATATATTTTTTGCATTGTTCATTTTTTTCTTTTACTTAATC
TTCATTGCTTTGAAATTAATAAAACAAAAAGAAGGACCGAATAGTTTGA
AGTTTGAACTATTGCCTATTCATGTAACTTATTCACCCAATCTTATATA
GTTTTTCTGGTAGAGATCATTTTAAATTGAAGGATATAAATTAAGAGGA
AATACTTGTATGTGATGTGTGGCAATTTGGAAGATCATGCGTAGAGAGT
TTAATGGCAGGTTTTGCAAATTGACCTGTAGTCATAATTACACTGGGCC
CTCTCGGAGTTTTGTGCCTTTTTGTTGTCGCTGTGTTTGGTTCTGCATG
TTAGCCTCACACAGATATTTAGTAGTTGTTGTTCTGCATATAAGCCTCA
CACGTATACTAAACGAGTGAACCTCAAAATCATGGCCTTACACCTATTG
AGTGAAATTAATGAACAGTGCATGTGAGTATGTGACTGTGACACAACCC
CCGGTTTTCATATTGCAATGTGCTACTGTGGTGATTAACCTTGCTACAC
TGTCGTCCTTGTTTGTTTCCTTATGTATATTGATACCATAAATTATTAC
TAGTATATCATTTTATATTGTCCATACCATTACGTGTTTATAGTCTCTT
TATGACATGTAATTGAATTTTTTAATTATAAAAAATAATAAAACTTAAT
TACGTACTATAAAGAGATGCTCTTGACTAGAATTGTGATCTCCTAGTTT
CCTAACCATATACTAATATTTGCTTGTATTGATAGCCCCTCCGTTCCCA
AGAGTATAAAACTGCATCGAATAATACAAGCCACTAGGCATGGTAAATT
AAATTGTGCCTGCACCTCGGGATATTTCATGTGGGGTTCATCATATTTG
TTGAGGAAAAGAAACTCCCGAAATTGAATTATGCATTTATATATCCTTT
TTCATTTCTAGATTTCCTGAAGGCTTAGGTGTAGGCACCTAGCTAGTAG
CTACAATATCAGCACTTCTCTCTATTGATAAACAATTGGCTGTAATGCC
GCAGTAGAGGACGATCACAACATTTCGTGCTGGTTACTTTTTGTTTTAT
GGTGGCAACAGCTGCTACTTCATCATTTTTCCCTGTTACTTCACCCTCG
CCGGACTCTGGTGGAGCAGGCAGCAAACTTGGTGGTGGGCCTGCAAACC
TTGGAGGACTAAAATCCAAATCTGCGTCTTCTGGTGGCTTGAAGGCAAA
GGCGCAAGCCCCTTCGAAAATTAATGGAACCACAGTTGTTACATCTAAA
GAAAGCTTCAAGCATGATGATGATCTACCTTCGCCTCCCCCCAGAACTT
TTATCAACCAGTTGCCTGATTGGAGCATGCTTCTTGCTGCTATCACAAC
AATTTTCTTGGCCGCTGAAAAGCAGTGGATGATGCTTGATTGGAAGCCA
CGGCGACCTGACATGCTTATTGACCCCTTTGGGATAGGAAAAATTGTTC
AGGATGGTCTTGTGTTCCGTGAAAACTTTTCTATTAGATCATATGAGAT
TGGTGCTGATCGTACCGCATCTATAGAAACAGTAATGAACCATTTGCAA
GTAAGTCCGTCCTCATACAAGTGAATCTTTATGATCTTCAGAGATGAGT
ATGCTTTGACTAAGATAGGGCTGTTTATTTAGTCACTGTAATTCAATTT
CATATATAGATAATATCATTG
82) Artificial sequence for tissue-specific
expression of SACPD-C and FATB-1A genes
(SEQ ID NO: 82)
AGAAAAAATGAATTTATTTATTTTTTGAAAGGTATACAGCTCTAAGACA
AGTAGTGTCCAAACCTGCATGACAACAAAGAATAAAAACCCTTCCGAGA
CACAACCCCTTTACAATTACATGCAACACCCCTAAACAATTATTATTTG
AATCTCCTCTCGTAAGAAAAATGTAAATAGCAAGTTTACCTCATTAGCA
AGTCTACTTTCACACGTTTGGGATTTCAAACATACTTTTTAATATATTA
TTTAATGGTTTTTTCCCTTAATTTTATAGTTATTAATAAATTTTAACTA
ATAATAAAATGTGTTAGAAATAATATATTAAAAAGTGTATTATTAGCCT
TTTTTTTTGTTTTTCAGGAAAATTGATTTTACAACTTTAACTAAAAAGA
TCTTGGAGAATATATAGATAATGGAAAAGATTGGAATGAATAAATAAGA
TATTAAGGTTAGCCAACAAATTAGTAGTTCTGGATCTGAGAACAATCAA
CTCTGTCACAAGGTGACCCGTATTTAACCTCCTAATAGATCCAATTCGT
AAGTGACGACATTACCAATTTACCAACAACAGTTACAAGCGAAAGTTAG
TGACATTACCAACGATCAATATACATTTTTTTTTACAAAACTATTTATG
CATACATGATATAATGCTTTATTGGGTAAACATCGATCCTAATAGTAGA
TCCGCAAATCTGATACATTATACAAAGCTCTCTAGTGAGAAAACATCCT
AATCGTAATTATAAAAGAAAAATACAAGAATTTATTGTATAAAAAGAAA
TTGGCATTGTCGATTTGTCGTATGCACCAACTCAAACTCAAATATATAG
AATTATATTGTATTCCTAAAACAATAAGCAGGGAAGTAACTGTGCGAGT
GCAACGAGAATAATAACAAAAGACAAGAATTTATTAAGCAAATAGAAAC
CTTGAGCTAGCCGTTTATTTATTTAATTTGAGTCGTACGTACGGCATAT
AATGATGGTTGAAAGGCTAGTCACAAAAGCAATTCTTTATTGAAAATCC
AACTGAACTTAACGCCATGATGCTTCTTCATCTTACGCGCTCGCTCATC
AGCGCGCTCTTGCAACCTCCTAATCCTCGGCGCCAACCCACACACGAAA
TCCTGCGCCCGCTTCCCCTCAGCCATCAATCCTTCAAGCTTCTCCAATC
TCCACCGTTCAACGAGAAACTCCAAGATGTCTGCGTAGTCGTTGGCGGT
GTACACGCCTATGCGCTGCGCCACAGCGGAGTAGTGCTCGAATAGCCTG
GGGTCATCCCCATCGTACATAAGGTGCGCCGGCATCGTGATCTTCTTCT
CCATCATGTTCCCTATGGCCACCATTGCCCCGGTGGGGTCCACTTCCAG
AAGCTTCTCCACGATTCTTGAGTACGCGTTCTCGTGCCGCTTCTCGTCC
GCTGCGATGGTCCCGCATAGGCGCGCCAGCACTGGATCCCCGCCCTCCT
TCGCGAGCCGAGCCGTGTTCCCGTGCGCCACAAATGTTGCTCGCTCTTG
GAATGACGTGTACACAAACCCCAAATATGGGTTGTTGTCTGTCCCAGGG
TCCATGCCAGCTGAAATGAGGTAATGTACGGTCTTTTCGACCTTAGCCA
TGTCAACCCTCCCAGAGAGATACAAATAAGTTCTGAGCAGATCCCCGTG
TCTGTTTTCCTCGGCGGTCCAGGCCCGGGTCCACACGGCCCACGGGCTC
GGGCTCGTGCCGCTGTCATCTTTCACTCCATCAAGGTTGTTGATCATGG
TCTGGTAAGTGGGAAGCGCGTCCTCGGTGACCATATCACCCACCAGCAC
CACAAAGTACTCATCAGGTAACTCTTTAGTGCGTTCGCGAAGCTCCTTC
ACCTGATGGCTGAACTCTTCATGCGGAAGGGAGGGGTCAGGGAGGAAGT
TTTGTGGCTGCCAGCATTGCTCCACGGGCTTCAGCAGCGGTAGGACCCA
CTCCGAGGCCCATCCCTCCAAGGACTTGAAAATTTCTTTCTTTTCTGGA
GGCATCAAATTAATTTTCAGCTTTCTCTCTTAATTTTTCTGTTTTAGTG
GAACATGTTAACGTTAAGCAACACACTTTGTCTTTGATCTAGCTTGGCT
TGGCTATAAATGCTAATGTAAAGCCAACCAGGAGCAGGAACAAAGGGAG
AGAAATTTATTCGCCCCTGGTTTAGTGTGCTTTCTCTTGTTTTACAAAT
ATATTATTTTAAATGTGTAATGGAGATAAGCTTGTTAATTTTAAATATT
ATATATCTTCCTTTATCACATCCTTTTTTTTATTTTATTTTATTCATTT
TCCAACATAAACAAATATTATAATACTTTAAAACTTCTTTTAATAATTT
GTTATAATTGTTAAATAAAGTTATAATTGTCAATTAAAAATTATAAATA
AAAATTAAAATTACTACGTTTTGATTATCACACATATGTTCTATATTCT
TTATTACTTTAATAGATAAAGAGTAAAAAAATTGTTAATTAAAAATTAA
TAAATAGAATTATACCAAAAATACGTAAGTATATATAATAAATTATAAA
AATATTAAATTTTTAATAAAAATACGTAAGTACATAATATATAATAAAA
AATTAATAACTATTTTTAATTAATTATAAAAAGATACACTCTTTACCAC
ACCTTCTCTTGAGTCCTTCTAAGGCCTTAGGACTCCCTAGTGGGTGGTA
TTCTCTAACATGATGAACACTTTATGTTTTAATGTTATGCACCTGTACT
AGTATTAGTTCTCTAGAACTTTAAAGTGCAGTCCTGGCCTGCGAATCCA
TCACTTTTAGGGATAAATAGTGATAGCCTTTTCATCATTAAAAAACGAA
AAAAGTATAATGAGTTGGAAAAACAAATATTTATTAAAATAGATGATTT
TTGTAACAGGATATATCATTTTAATATAATCCATCAAGCCAACACAAAA
TGAATATTAGACCAGGTGCCTCTTATATTTCTACAGTAACAAAAATAAT
TTATTTTAATAAATGTTTTCTTGATCAACATATTATAATAATTAGTTTT
CGTTTTTAACTTGTTTTTGGTGAAAAAGCCAAATATTGGACCAGGTGCA
GAAATAGAGATAAGACTATGGAGCCCTAACCGCGCGCGAGTGAGATTTC
TTCCCTTATGAGCAGATTTTTGATAAATGCTGAACGACCTTTGCCATAA
TGAACAACATGGTCCCCACAATGCTCAGTTTTATAACCTCTTGGATGAA
ATCAGAAAGGTAATGATCTTCTGCTCCCTGACAATATATTTGTTAATGC
TGTCTAATGTCTACACTTAACTATAAATGCATTCTTCTAGTTCATTAGA
CATTTACTATATTACATGTGCTTCGCAATGGTATCTGTTCACCTTGTGC
AAGAGGTTTTCAATTGAATTGAGATATTGAAAATTGTCCAATTAATTTA
TGGAATTATGGTAATATGCTATATTGTTTGTGTTGACCTGCTATCTATG
TATATATATGCACATTCCATCCTTATTGGTATTAGTTCAGAGGACAAAG
AAATTAGTAGCACTGGGAAAGGATTTGAGCTTAGTCATCATACTTGGCC
AGACTGCATTGGCTGCTGCTGAAAGCAGAGCACATCATAGAGTTAATTA
GAGCACACCAGTATTTAGCTCAGGTGGCCACAATAATATTAAGTCTGCT
CTGAGTCCAATACTTCTGAAAGGAGATGACATGTGGTGTGGATCAAACG
TCAAAGTCTCTAGGGCCGGGGAATCTGCAACAGCCAAGGAAACATTTGA
GTTCCGTGGGCCTTACATCATTTTAGCACAATACTTGCATCTATAGTAT
GAAATTTTTGTGACAAATTAGAAATGTAATTTAGGAAATGTTCTTACCA
CTGAAGTGCTCTTCAAATCGATCTCCACCAATAATCTAATCTTCTACTT
GGTATTCTAAGCTTTTTCGGGTGGTGGGAGTGGTACATGTAAAGGTACT
CTGACGCTTAAGTCAGTCAATAATAGAATTATAATAAAGTTAGAATGCG
TCAACATTTATATAATATATAGAAAAAAATTTGAAATTAATCACAAAAA
CTAAAATTAAGAATTTGTCTAAAATAAGAATAAAGTATCTCAATTAAAA
AATAAAAACTAAAATCACAAATTTTAAAAAAGTGAAGGATAAAATGTAT
CATTTAAAAAATGGGAAAACGAAAATCACATATTTAAAAAAATAAGAGA
TAGAAATTGCATTTTAATATTTTTTTTTATTTCTCTTCCTTTTTTAATT
ATACTTTTAATCACATTAATGATTTTATTTTCTATTTCTCTTCTTTCCA
CCTACATACATCCCAAAGATGGAGGGTGCAATTGTAAGTTTATTAGCAC
TCTTGTTTTTACCTGCATTTGTGTGTGCTAACCAAATTGCATTCTTCTC
TTTACATAATGTATTTGATTTGAATTTTCATACCACATGCAAGCATGAT
TACGTACGTGTCCATGATCAAATACAAATGCTGTCTGGTACTGGCAATT
TGGTAAACAGCCATCCATTTTTTTTTGTCTCTAATTATTCTCTAGAATA
TCTGAAGATTCCTCTGTCATCGAATTCCTTGCTTGGTAACAACGTCGTC
AAGTTATTATTTTGTTCTTTTTTTTTTTATCATATTTCTTATTTTGTTC
CAAGTATGTCATATTTTGATCCATCTTGACAAGTAGATTGTCATGTAGG
AATAGGAATATCACTTTAAATTTTAAAGCATTGATTAGTCTGTAGGCAA
TATTGTCTTCTTCTTCCTCCTTATTAATATTTTTTATTCTGCCTTCAAT
CACCAGTTATGGGAGATGGATGTAATACTAAATACCATAGTTGTTCTGC
TTGAAGTTTAGTTGTATAGTTGTTCTGCTTGAAGTTTAGTTGTGTGTAA
TGTTTCAGCGTTGGCTTCCCCTGTAACTGCTACAATGGTACTGAATATA
TATTTTTTGCATTGTTCATTTTTTTCTTTTACTTAATCTTCATTGCTTT
GAAATTAATAAAACAAAAAGAAGGACCGAATAGTTTGAAGTTTGAACTA
TTGCCTATTCATGTAACTTATTCACCCAATCTTATATAGTTTTTCTGGT
AGAGATCATTTTAAATTGAAGGATATAAATTAAGAGGAAATACTTGTAT
GTGATGTGTGGCAATTTGGAAGATCATGCGTAGAGAGTTTAATGGCAGG
TTTTGCAAATTGACCTGTAGTCATAATTACACTGGGCCCTCTCGGAGTT
TTGTGCCTTTTTGTTGTCGCTGTGTTTGGTTCTGCATGTTAGCCTCACA
CAGATATTTAGTAGTTGTTGTTCTGCATATAAGCCTCACACGTATACTA
AACGAGTGAACCTCAAAATCATGGCCTTACACCTATTGAGTGAAATTAA
TGAACAGTGCATGTGAGTATGTGACTGTGACACAACCCCCGGTTTTCAT
ATTGCAATGTGCTACTGTGGTGATTAACCTTGCTACACTGTCGTCCTTG
TTTGTTTCCTTATGTATATTGATACCATAAATTATTACTAGTATATCAT
TTTATATTGTCCATACCATTACGTGTTTATAGTCTCTTTATGACATGTA
ATTGAATTTTTTAATTATAAAAAATAATAAAACTTAATTACGTACTATA
AAGAGATGCTCTTGACTAGAATTGTGATCTCCTAGTTTCCTAACCATAT
ACTAATATTTGCTTGTATTGATAGCCCCTCCGTTCCCAAGAGTATAAAA
CTGCATCGAATAATACAAGCCACTAGGCATGGTAAATTAAATTGTGCCT
GCACCTCGGGATATTTCATGTGGGGTTCATCATATTTGTTGAGGAAAAG
AAACTCCCGAAATTGAATTATGCATTTATATATCCTTTTTCATTTCTAG
ATTTCCTGAAGGCTTAGGTGTAGGCACCTAGCTAGTAGCTACAATATCA
GCACTTCTCTCTATTGATAAACAATTGGCTGTAATGCCGCAGTAGAGGA
CGATCACAACATTTCGTGCTGGTTACTTTTTGTTTTATGGTGGCAACAG
CTGCTACTTCATCATTTTTCCCTGTTACTTCACCCTCGCCGGACTCTGG
TGGAGCAGGCAGCAAACTTGGTGGTGGGCCTGCAAACCTTGGAGGACTA
AAATCCAAATCTGCGTCTTCTGGTGGCTTGAAGGCAAAGGCGCAAGCCC
CTTCGAAAATTAATGGAACCACAGTTGTTACATCTAAAGAAAGCTTCAA
GCATGATGATGATCTACCTTCGCCTCCCCCCAGAACTTTTATCAACCAG
TTGCCTGATTGGAGCATGCTTCTTGCTGCTATCACAACAATTTTCTTGG
CCGCTGAAAAGCAGTGGATGATGCTTGATTGGAAGCCACGGCGACCTGA
CATGCTTATTGACCCCTTTGGGATAGGAAAAATTGTTCAGGATGGTCTT
GTGTTCCGTGAAAACTTTTCTATTAGATCATATGAGATTGGTGCTGATC
GTACCGCATCTATAGAAACAGTAATGAACCATTTGCAAGAAACTGCACT
TAATCATGTTAAAAGTGCTGGGCTTCTTGGTGATGGCTTTGGTTCCACG
CCAGAAATGTGCAAAAAGAACTTGATATGGGTGGTTACTCGGATGCAGG
TTGTGGTGGAACGCTATCCTACATGGGGTGACATAGTTCAAGTGGACAC
TTGGGTTTCTGGATCAGGGAAGAATGGTATGCGCCGTGATTGGCTTTTA
CGTGACTGCAAAACTGGTGAAATCTTGACAAGAGCTTCCAGTGTTTGGG
TCATGATGAATAAGCTAACACGGAGGCTGTCTAAAATTCCAGAAGAAGT
CAGACAGGAGATAGGATCTTATTTTGTGGATTCTGATCCAATTCTGGAA
GAGGATAACAGAAAACTGACTAAACTTGACGACAACACAGCGGATTATA
TTCGTACCGGTTTAAGTCCTAGGTGGAGTGATCTAGATATCAATCAGCA
TGTCAACAATGTGAAGTACATTGGCTGGATTCTGGAGAGTGCTCCACAG
CCAATCTTGGAGAGTCATGAGCTTTCTTCCATGACTTTAGAGTATAGGA
GAGAGTGTGGTAGGGACAGTGTGCTGGATTCCCTGACTGCTGTATCTGG
GGCCGACATGGGCAATCTAGCTCACAGCGGGCATGTTGAGTGCAAGCAT
TTGCTTCGACTGGAAAATGGTGCTGAGATTGTGAGGGGCAGGACTGAGT
GGAGGCCCAAACCTGTGAACAACTTTGGTGTTGTGAACCAGGTTCCAGC
AGAAAGCACCTAATGGAGCAACCAATGGGCCATAGTGGGAGTTATGGAA
GTTTTGTCATGTATTAGTACATAATTAGTAGAATGTTATAAATAAGTGG
ATTTGCCGCGTAATGACTTTGTGTGTATTGTGAAACAGCTTGTTGCGAT
CATGGTTATAATGTAAAAATAATTCTGGTATTAATTACATGTGGAAAGT
GTTCTGCTTATAGCTTTCTGCCTAAAATGCACGCTGCACGGGACAATAT
CATTGGTAATTTTTTTAAAATCTGAATTGAGGCTACTCATAATACTATC
CATAGGACATCAAAGACATGTTGCATTGACTTTAAGCAGAGGTTCATCT
AGAGGATTACTGCATAGGCTTGAACTACAAGTAATTTAAGGGACGAGAG
CAACTTTAGCTCTACCACGTCGTTTTACAAGGTTATTAAAATCAAATTG
ATCTTATTAAAACTGAAAATTTGTAATAAAATGCTATTGAAAAATTAAA
ATATAGCAAACACCTAAATTGGACTGATTTTTAGATTCAAATTTAATAA
TTAATCTAAATTAAACTTAAATTTTATAATATATGTCTTGTAATATATC
AAGTTTTTTTTTTTATTATTGAGTTTGGAAACATATAATAAGGAACATT
AGTTAATATTGATAATCCACTAAGATCGACTTAGTATTACAGTATTTGG
ATGATTTGTATGAGATATTCAAACTTCACTCTTATCATAATAGAGACAA
AAGTTAATACTGATGGTGGAGAAAAAAAAATGTTATTGGGAGCATATGG
TAAGATAAGACGGATAAAAATATGCTGCAGCCTGGAGAGCTAATGTATT
TTTTGGTGAAGTTTTCAAGTGACAACTATTCATGATGAGAACACAATAA
TATTTTCTACTTACCTATCCCACATAAAATACTGATTTTAATAATGATG
ATAAATAATGATTAAAA (SEQ ID NO: 82)

1. A soybean plant, plant part, or plant cell comprising one or more mutations modulating the expression of a SACPD-C gene, a FATB-1A gene, or both the SACPD-C and FATB-1 A genes, wherein said plant, plant part, or plant cell produces oil that has increased saturated fatty acid content as compared to oil produced from a corresponding soybean plant, plant part, or plant cell lacking the one or more mutations, and wherein the one or more mutations modulating the expression of the SACPD-C gene comprise a targeted mutation induced by a rare-cutting endonuclease.

2. The soybean plant, plant part, or plant cell of claim 1, wherein the soybean plant, plant part, or plant cell comprises a mutation resulting in reduced expression of the SACPD-C gene.

3. The soybean plant, plant part, or plant cell of claim 2, wherein the mutation resulting in reduced expression of the SACPD-C gene is a mutation in one or more alleles of the SACPD-C gene or an operatively linked promoter thereof.

4. The soybean plant, plant part, or plant cell of claim 2, wherein the mutation resulting in reduced expression of the SACPD-C gene is a knock-out mutation.

5. The soybean plant, plant part, or plant cell of claim 4, wherein the knock-out mutation is a seed-specific knock-out mutation.

6. The soybean plant, plant part, or plant cell of claim 5, wherein the seed-specific knock-out mutation comprises replacement of a seed-specific promoter at a native genomic locus of the SACPD-C gene with a promotor with low activity or no detectable activity in developing soybean seed or a knock-in mutation of a functional SACPD-C gene operably-linked to a promotor with low activity or no detectable activity in developing soybean seed.

7. The soybean plant, plant part, or plant cell of claim 2, wherein the mutation is in a sequence set forth in SEQ ID NO: 15, 18, 21, 24, 27, 30, 33, 36, 39, 42, or 45, or a sequence having at least 95% identity to a sequence set forth in SEQ ID NO: 15, 18, 21, 24, 27, 30, 33, 36, 39, 42, or 45.

8. The soybean plant, plant part, or plant cell of claim 6, comprising a knock-in mutation of a functional SACPD-C gene operably-linked to a promotor with low activity or no detectable activity in developing soybean seed.

9. The soybean plant, plant part, or plant cell of claim 6, wherein the promoter with low activity or no detectable activity in developing soybean seed is a nodule specific gene promoter.

10. The soybean plant, plant part, or plant cell of claim 1, wherein the soybean plant, plant part, or plant cell comprises a mutation resulting in increased expression of the FATB-1A gene.

11. The soybean plant, plant part, or plant cell of claim 10, wherein the mutation increasing expression of the FATB-1A gene is a targeted replacement of the endogenous promoter of the FATB-1A gene with an overexpression promoter.

12. The soybean plant, plant part, or plant cell of claim 11, wherein the promoter or overexpression promoter is a strong seed-specific promoter, optionally a FAD2A promoter or a FAD2B promoter.

13. A method for generating a soybean plant comprising a mutation modulating the expression of a SACPD-C gene, a FATB-1A gene, or both the SACPD-C and FATB-1A genes, comprising:
(a) contacting a population of soybean plant cells from a soybean plant producing an oil with a saturated fatty acid content of about 15% of total fatty acid composition with one or more nucleic acid sequences inducing:
(i) a mutation resulting in reduced expression of the SACPD-C gene, wherein the mutation is a targeted mutation induced by a rare cutting endonuclease;
(ii) a mutation resulting in increased expression of the FATB-1A gene; or
(iii) a combination thereof;
(b) selecting, from the population, a cell in which expression of the SACPD-C gene has been reduced, expression of the FATB-1A gene has been increased, or expression of the SACPD-C gene has been reduced and expression of the FATB-1A gene has been increased, and
(c) regenerating the selected plant cell into a soybean plant.

14. The method of claim 13, wherein reducing expression of the SACPD-C gene comprises inducing a mutation in one or more alleles of the SACPD-C gene or an operatively linked promoter thereof, optionally wherein the induced mutation is a knock out mutation or a seed-specific knock-out mutation.

15. The method of claim 14, wherein reducing expression of the SACPD-C gene comprises replacing a seed-specific promoter at a native genomic locus of the SACPD-C gene with a promotor with low activity or no detectable activity in developing soybean seed.

16. The method of claim 14, further comprising delivering to the population of soybean plant cells an expression cassette comprising a functional SACPD-C gene operably-linked to a promotor with low activity or no detectable activity in developing soybean seed, and optionally operably linked to a nodule specific promoter.

17. The method of claim 13, wherein increasing expression of the FATB-1A gene comprises replacing an endogenous promoter of the FATB-1A gene with an overexpression promoter.

18. The method of claim 13, wherein increasing expression of the FATB-1A gene comprises delivering to the population of soybean plant cells an expression cassette comprising one or more copies of the FATB-1A gene, optionally operably linked to a strong seed-specific promoter.

19. A soybean oil composition, comprising a soybean oil produced by a soybean plant, plant part, or plant cell comprising one or more mutations modulating the expression of a SACPD-C gene, a FATB-1A gene, or both the SACPD-C and FATB-1A genes, wherein the soybean oil has increased saturated fatty acid content as compared to oil produced from a corresponding soybean plant, plant part, or plant cell lacking the one or more mutations and wherein the one or more mutations modulating the expression of the SACPD-C gene comprise a targeted mutation induced by a rare-cutting endonuclease.

20. The soybean oil composition of claim 19, wherein the soybean oil has a stearic acid content of greater than 10%, a palmitic acid content of greater than 10%, or a saturated fatty acid content of greater than 20%, wherein all percentages are based on the weight of the total fatty acids of the oil.


SEQUENCE LISTING (82 sequences)

SEQ ID NO: 1 - DNA, 1014 bp, Glycine max
atgcctccagaaaagaaagaaattttcaagtccttggagggatgggcctcggagtgggtcctaccgctgctgaagcccgtggagcaatgctggcagccacaaaacttcctccctgacccctcccttccgcatgaagagttcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgagcccgagcccgtgggccgtgtggacccgggcctggaccgccgaggaaaacagacacggggatctgctcagaacttatttgtatctctctgggagggttgacatggctaaggtcgaaaagaccgtacattacctcatttcagctggcatggaccctgggacagacaacaacccatatttggggtttgtgtacacgtcattccaagagcgagcaacatttgtggcgcacgggaacacggctcggctcgcgaaggagggcggggatccagtgctggcgcgcctatgcgggaccatcgcagcggacgagaagcggcacgagaacgcgtactcaagaatcgtggagaagcttctggaagtggaccccaccggggcaatggtggccatagggaacatgatggagaagaagatcacgatgccggcgcaccttatgtacgatggggatgaccccaggctattcgagcactactccgctgtggcgcagcgcataggcgtgtacaccgccaacgactacgcagacatcttggagtttctcgttgaacggtggagattggagaagcttgaaggattgatggctgaggggaagcgggcgcaggatttcgtgtgtgggttggcgccgaggattaggaggttgcaagagcgcgctgatgagcgagcgcgtaagatgaagaagcatcatggcgttaagttcagttggattttcaataaagaattgcttttgtga

SEQ ID NO: 2 - DNA, 2000 bp, Glycine max
ctaaaatgaaattaaaatataaaatatataaattaaaaatatcatttttaaattataaagaaaaaaaatcacttttaaaattataaagactaaaaaaaattaaaatataaagggaaaaaattcaattccaaacgataaaaaaacaaaaaaaaaatatcttcaaactatagaaataatttaagttcccgaatttgaatctagttaagttgaaataaaaaataaaaatttatatcaaatttgattcagctcccagaagaggcagtatataatttcatatacaccaagcgccaagacgcaagccgttagttacgaggtccttttccgtttgtcagtatcaaaccccttttttaatttcattttcttattttttggcataaaaaatttacagcagcgagagataaatcccatgccactctccagcgagagatatacttaatttgaacatttgtgtgtactttaaatgcaagaagtgataatgtcttgatcaaaaataaaaatgtaagaagtgataactccccagcccataaccagagtttgatttctaattataccaataaaacaatacttataggtaaaggaattgttgttaactaatactttactttttaaaattataaaaatatttctgaatttaatcttagatcttaaaaaatgaaaaaaaaactataaaataatgttttattgttagtattttaaaattataatattatattttataataataataataattagaaaaaaaccgctcttaagtcatttaacaataataataatgttttattattattattatttctgatttgactatggttgaataaattactatattgataaatctcttattgagaaaaaaaaaccattcttaagtcatttaacaataagaatatttcaaatattaataaaaaaaaaccaaacatcaattaagaaactgaatgtttatttttaataactaaaatttaaataataaatacttttgattatataagctatattataatctaaatttataatacaaataattttaaatatataatttatatttaatttatgtaatatatataaatatattctaaaatttataataaaaaagttataatataatataaaattattttaaattactaataacttcttttaaatatgttaaattttaatttaaaaatagagatgagaatgataaaatataagagatataaacagttaaaaaatttcttaaaattattctagtcgaatattattattctaatttatatttatagaaagattatcataccaaaaaaattgctcaaaaaaattattataccaaaaaaatatatatagaaaaattattatcacatccatgtcaagttcacggttttgtcggggatggtggattggtttagacttataaagtcgtcttttgctggccagcacttcgtaaatccacttatctgaaattgtgaacagtattgttcttgcgttgtttcggagggtcaattgtttggttgagcaaaaatctgaaattcaatttcaggtctttaacttttaactttcttggttgatatctggagggattgcttccaaaactctgcattgtctgtgtgtaatggttgttagagtttacccaattggctaattatttatatatttttatgtttggactttgtctttgtgttcccttgttccacacataaattaaacaaaggtactatatactacttttctaggttgtatctgcatgacaacaatgatatctccttgaagaggttgtgattggcttgtcatgccagttagcttgcaattctgggtttctgacagacagtatttaaattgtatttatgtatatgactatgactttggaatgtatacggctaaaacatatacgtcatgattaatggtggtgatgaatatatcaagaatagcaagtatttgtgtactctcagtattcgagagctacttgcttataatttatgcaagaactctcctaagtcagctttgctttcgaagcctttatctttgag

SEQ ID NO: 3 - DNA, 2000 bp, Glycine max
ttctaactttattataattctattattgactgacttaagcgtcagagtacctttacatgtaccactcccaccacccgaaaaagcttagaataccaagtagaagattagattattggtggagatcgatttgaagagcacttcagtggtaagaacatttcctaaattacatttctaatttgtcacaaaaatttcatactatagatgcaagtattgtgctaaaatgatgtaaggcccacggaactcaaatgtttccttggctgttgcagattccccggccctagagactttgacgtttgatccacaccacatgtcatctcctttcagaagtattggactcagagcagacttaatattattgtggccacctgagctaaatactggtgtgctctaattaactctatgatgtgctctgctttcagcagcagccaatgcagtctggccaagtatgatgactaagctcaaatcctttcccagtgctactaatttctttgtcctctgaactaataccaataaggatggaatgtgcatatatatacatagatagcaggtcaacacaaacaatatagcatattaccataattccataaattaattggacaattttcaatatctcaattcaattgaaaacctcttgcacaaggtgaacagataccattgcgaagcacatgtaatatagtaaatgtctaatgaactagaagaatgcatttatagttaagtgtagacattagacagcattaacaaatatattgtcagggagcagaagatcattacctttctgatttcatccaagaggttataaaactgagcattgtggggaccatgttgttcattatggcaaaggtcgttcagcatttatcaaaaatctgctcataagggaagaaatctcactcgcgcgcggttagggctccatagtcttatctctatttctgcacctggtccaatatttggctttttcaccaaaaacaagttaaaaacgaaaactaattattataatatgttgatcaagaaaacatttattaaaataaattatttttgttactgtagaaatataagaggcacctggtctaatattcattttgtgttggcttgatggattatattaaaatgatatatcctgttacaaaaatcatctattttaataaatatttgtttttccaactcattatacttttttcgttttttaatgatgaaaaggctatcactatttatccctaaaagtgatggattcgcaggccaggactgcactttaaagttctagagaactaatactagtacaggtgcataacattaaaacataaagtgttcatcatgttagagaataccacccactagggagtcctaaggccttagaaggactcaagagaaggtgtggtaaagagtgtatctttttataattaattaaaaatagttattaattttttattatatattatgtacttacgtatttttattaaaaatttaatatttttataatttattatatatacttacgtatttttggtataattctatttattaatttttaattaacaatttttttactctttatctattaaagtaataaagaatatagaacatatgtgtgataatcaaaacgtagtaattttaatttttatttataatttttaattgacaattataactttatttaacaattataacaaattattaaaagaagttttaaagtattataatatttgtttatgttggaaaatgaataaaataaaataaaaaaaaggatgtgataaaggaagatatataatatttaaaattaacaagcttatctccattacacatttaaaataatatatttgtaaaacaagagaaagcacactaaaccaggggcgaataaatttctctccctttgttcctgctcctggttggctttacattagcatttatagccaagccaagctagatcaaagacaaagtgtgttgcttaacgttaacatgttccactaaaacagaaaaattaagagagaaagctgaaaattaatttg

SEQ ID NO: 4 - DNA, 903 bp, Glycine max
cctctactccacctagatcttgtatttggtttgtatgggagtatgtttgaagctatagcgcctgtggttgtatacctgtatttctgtgcagtgtgttttgtgattttgttttagaataaactgcaaaattgatcttccaaagattatggcatcacctcattaatgtttttaagatttttgttatcaaattagtcccacaaatatctaaaatgttaccacatttgttcacatagaggactaaagaagtggtaacacaaaataatttagtatttgatttgtatctctgtgggattgatttgttgaccaaaatctttggaggaccagttcaaggacttactctatcgatttatactttcagtttcaggcagtcaagtaatactgtatattctgttgtctatattgtggatcatgcataactaaactatcaagtatccctgtatatacaagttgtctaaattgtgaaagttacatatacaagaaatttcctatataccgttagagagtagataaactatataccgttagagagttttcatgctaattgttaaattaaatactacgctttctaaccttccatcttttgaaccaataaaaaatgtttctactcatcgtattctccattaagttgaaatattaccaattaagttatgttacaaaattttccctcggaatctgaaggtttgcttgtatagcagtgctcatttttttctaaaagaaaatatacaattgcttcccctttacgaatagtaaagtcattcacaacgaagtaaagaacagaagtaacacagcagctaccatctcatccaagaaaaaaaaaaagtaattctggagctatttgtttcagatattcaacttctaaaagaatattaaacttaaaacaggtaaaaatgataaacaaatacc

SEQ ID NO: 5 - DNA, 1000 bp, Glycine max
ctagcctttcaaccatcattatatgccgtacgtacgactcaaattaaataaataaacggctagctcaaggtttctatttgcttaataaattcttgtcttttgttattattctcgttgcactcgcacagttacttccctgcttattgttttaggaatacaatataattctatatatttgagtttgagttggtgcatacgacaaatcgacaatgccaatttctttttatacaataaattcttgtatttttcttttataattacgattaggatgttttctcactagagagctttgtataatgtatcagatttgcggatctactattaggatcgatgtttacccaataaagcattatatcatgtatgcataaatagttttgtaaaaaaaaatgtatattgatcgttggtaatgtcactaactttcgcttgtaactgttgttggtaaattggtaatgtcgtcacttacgaattggatctattaggaggttaaatacgggtcaccttgtgacagagttgattgttctcagatccagaactactaatttgttggctaaccttaatatcttatttattcattccaatcttttccattatctatatattctccaagatctttttagttaaagttgtaaaatcaattttcctgaaaaacaaaaaaaaaggctaataatacactttttaatatattatttctaacacattttattattagttaaaatttattaataactataaaattaagggaaaaaaccattaaataatatattaaaaagtatgtttgaaatcccaaacgtgtgaaagtagacttgctaatgaggtaaacttgctatttacatttttcttacgagaggagattcaaataataattgtttaggggtgttgcatgtaattgtaaaggggttgtgtctcggaagggtttttattctttgttgtcatgcaggtttggacactacttgtcttagagctgtatacctttcaaaaaataaataaattcattttttct

SEQ ID NO: 6 - DNA, 1251 bp, Glycine max
atggtggcaacagctgctacttcatcatttttccctgttacttcaccctcgccggactctggtggagcaggcagcaaacttggtggtgggcctgcaaaccttggaggactaaaatccaaatctgcgtcttctggtggcttgaaggcaaaggcgcaagccccttcgaaaattaatggaaccacagttgttacatctaaagaaagcttcaagcatgatgatgatctaccttcgcctccccccagaacttttatcaaccagttgcctgattggagcatgcttcttgctgctatcacaacaattttcttggccgctgaaaagcagtggatgatgcttgattggaagccacggcgacctgacatgcttattgacccctttgggataggaaaaattgttcaggatggtcttgtgttccgtgaaaacttttctattagatcatatgagattggtgctgatcgtaccgcatctatagaaacagtaatgaaccatttgcaagaaactgcacttaatcatgttaaaagtgctgggcttcttggtgatggctttggttccacgccagaaatgtgcaaaaagaacttgatatgggtggttactcggatgcaggttgtggtggaacgctatcctacatggggtgacatagttcaagtggacacttgggtttctggatcagggaagaatggtatgcgccgtgattggcttttacgtgactgcaaaactggtgaaatcttgacaagagcttccagtgtttgggtcatgatgaataagctaacacggaggctgtctaaaattccagaagaagtcagacaggagataggatcttattttgtggattctgatccaattctggaagaggataacagaaaactgactaaacttgacgacaacacagcggattatattcgtaccggtttaagtcctaggtggagtgatctagatatcaatcagcatgtcaacaatgtgaagtacattggctggattctggagagtgctccacagccaatcttggagagtcatgagctttcttccatgactttagagtataggagagagtgtggtagggacagtgtgctggattccctgactgctgtatctggggccgacatgggcaatctagctcacagcgggcatgttgagtgcaagcatttgcttcgactggaaaatggtgctgagattgtgaggggcaggactgagtggaggcccaaacctgtgaacaactttggtgttgtgaaccaggttccagcagaaagcacctaa

SEQ ID NO: 7 - DNA, 2000 bp, Glycine max
tgcgtcaacatttatataatatatagaaaaaaatttgaaattaatcacaaaaactaaaattaagaatttgtctaaaataagaataaagtatctcaattaaaaaataaaaactaaaatcacaaattttaaaaaagtgaaggataaaatgtatcatttaaaaaatgggaaaacgaaaatcacatatttaaaaaaataagagatagaaattgcattttaatatttttttttatttctcttccttttttaattatacttttaatcacattaatgattttattttctatttctcttctttccacctacatacatcccaaagatggagggtgcaattgtaagtttattagcactcttgtttttacctgcatttgtgtgtgctaaccaaattgcattcttctctttacataatgtatttgatttgaattttcataccacatgcaagcatgattacgtacgtgtccatgatcaaatacaaatgctgtctggtactggcaatttggtaaacagccatccatttttttttgtctctaattattctctagaatatctgaagattcctctgtcatcgaattccttgcttggtaacaacgtcgtcaagttattattttgttctttttttttttatcatatttcttattttgttccaagtatgtcatattttgatccatcttgacaagtagattgtcatgtaggaataggaatatcactttaaattttaaagcattgattagtctgtaggcaatattgtcttcttcttcctccttattaatattttttattctgccttcaatcaccagttatgggagatggatgtaatactaaataccatagttgttctgcttgaagtttagttgtatagttgttctgcttgaagtttagttgtgtgtaatgtttcagcgttggcttcccctgtaactgctacaatggtactgaatatatattttttgcattgttcatttttttcttttacttaatcttcattgctttgaaattaataaaacaaaaagaaggaccgaatagtttgaagtttgaactattgcctattcatgtaacttattcacccaatcttatatagtttttctggtagagatcattttaaattgaaggatataaattaagaggaaatacttgtatgtgatgtgtggcaatttggaagatcatgcgtagagagtttaatggcaggttttgcaaattgacctgtagtcataattacactgggccctctcggagttttgtgcctttttgttgtcgctgtgtttggttctgcatgttagcctcacacagatatttagtagttgttgttctgcatataagcctcacacgtatactaaacgagtgaacctcaaaatcatggccttacacctattgagtgaaattaatgaacagtgcatgtgagtatgtgactgtgacacaacccccggttttcatattgcaatgtgctactgtggtgattaaccttgctacactgtcgtccttgtttgtttccttatgtatattgataccataaattattactagtatatcattttatattgtccataccattacgtgtttatagtctctttatgacatgtaattgaattttttaattataaaaaataataaaacttaattacgtactataaagagatgctcttgactagaattgtgatctcctagtttcctaaccatatactaatatttgcttgtattgatagcccctccgttcccaagagtataaaactgcatcgaataatacaagccactaggcatggtaaattaaattgtgcctgcacctcgggatatttcatgtggggttcatcatatttgttgaggaaaagaaactcccgaaattgaattatgcatttatatatcctttttcatttctagatttcctgaaggcttaggtgtaggcacctagctagtagctacaatatcagcacttctctctattgataaacaattggctgtaatgccgcagtagaggacgatcacaacatttcgtgctggttactttttgtttt

SEQ ID NO: 8 - DNA, 2000 bp, Glycine max
aacatattgggggtaccaaacaatttgcaccccataataaggaactgtggacaaaattgcatttgccacacctcccaatttatttagtagacgactcttccaagttccaagaagctaaccttgagtttatttgctcctcaatgtgattgaagtcagctctctgaattgcttcatgcacaatcttcaacttcgtgtttttttagttaccatatgaatctcccaatttatttagattgtacaataaattgagaaaactcaaataattaaatccaataattttttcttaaaaatcatttcaatctaattcactgtgaacacctttatctaaaattttacatgaaatttcaaatttaattcaattcttactaacataataatgctggatattttcttaattccaaataatactattaaatattagattcaatgtttataatatattccacaattgtattttttttatttgtaagaattaaaaataaatatcaaaaagtttacaacactcacatatcctaaccaatcaattgagttaaattccttatatagggattgtatgaattttcattttaatacatatatttaaaatcttattattgcaaaaaataataatctaatacttttttttaaaatacatgaatgtatgaccatgaaaaatggcaccatgttaaaaaaaactgttttaaatatgaatattttctctcataattaatattttttctatgcagtattgtttaaaaaaaaaaactgtttcttcaatttctgaaacaccgaaaaagagagaaagaaaaaattattgttttttaatttaatatggtgtgcttactcacaaagcagtcttacactaatctcgaaataaacttatcagatggtcgaaaatcctttggcacgttaaaacacgtcgtacaaaagatgcagctgatctgattctccacttgtctcaagcagacatcacactgatcacaggtggaaaccaaatttgcctaagttccaaggcctcggtgtgactcagccccaagtgacgccaaccaaacgcgtcctaactaaggtgtagaagaaacagatagtatataagtataccatataagaggagagtgagtggagaagcacttctccttttttttttctctgttgaaattgaaagtgttttccgggaaataaataaaataaattaaaatcttacacactctaggtaggtacttctaatttaatccacactttgactctatatatgttttaaaaataattataatgcgtacttactttctcattatactaaatttaacatcgatgattttattttctgtttctcttctttccacctacatacatcccaaaatttagggtgcaattttaagtttattaacacatgtttttagctgcatgctgcctttgtgtgtgctcaccaaattgcattcttctctttatatgttgtatttgaattttcacaccatatgtaaacaagattacgtacgtgtccatgatcaaatacaaatgctgtcttatactggcaatttgataaacagccgtccattttttctttttctctttaactatatatgctctagaatctctgaagattcctctgccatcgaatttctttcttggtaacaacgtcgtcgttatgttattattttattctatttttattttatcatatatatttcttattttgttcgaagtatgtcatattttgatcgtgacaattagattgtcatgtaggagtaggaatatcactttaaaacattgattagtctgtaggcaatattgtcttctttttcctcctttattaatatattttgtcgaagttttaccacaaggttgattcgctttttttgtccctttctcttgttctttttacctcaggtattttagtctttcatggattataagatcactgagaagtgtatgcatgtaatactaagcaccatagctgttctgcttgaatttatttgtgtgtaaattgtaatgtttcagcgttggctttccctgtagctgctaca

SEQ ID NO: 9 - DNA, 1000 bp, Glycine max
tggagcaaccaatgggccatagtgggagttatggaagttttgtcatgtattagtacataattagtagaatgttataaataagtggatttgccgcgtaatgactttgtgtgtattgtgaaacagcttgttgcgatcatggttataatgtaaaaataattctggtattaattacatgtggaaagtgttctgcttatagctttctgcctaaaatgcacgctgcacgggacaatatcattggtaatttttttaaaatctgaattgaggctactcataatactatccataggacatcaaagacatgttgcattgactttaagcagaggttcatctagaggattactgcataggcttgaactacaagtaatttaagggacgagagcaactttagctctaccacgtcgttttacaaggttattaaaatcaaattgatcttattaaaactgaaaatttgtaataaaatgctattgaaaaattaaaatatagcaaacacctaaattggactgatttttagattcaaatttaataattaatctaaattaaacttaaattttataatatatgtcttgtaatatatcaagtttttttttttattattgagtttggaaacatataataaggaacattagttaatattgataatccactaagatcgacttagtattacagtatttggatgatttgtatgagatattcaaacttcactcttatcataatagagacaaaagttaatactgatggtggagaaaaaaaaatgttattgggagcatatggtaagataagacggataaaaatatgctgcagcctggagagctaatgtattttttggtgaagttttcaagtgacaactattcatgatgagaacacaataatattttctacttacctatcccacataaaatactgattttaataatgatgataaataatgattaaaatatttgattctttgttaagagaaataaggaaaacataaatattctcatggaaaaatcagcttgta

SEQ ID NO: 10 - DNA, 1000 bp, Glycine max
tgaaccaagcaatgggccatagtgggagttatggaagttttgtcacttatcacttaattagtagaatgttataaataagtggatttgccgcgtaatgacttgtgtgcattgtgaaacagcttgtagcgatccatggctataatgtaaaaatatgtggaaagtgttctgcttataactttcttccttaaatgcacactccatagaacaatattattggtactaattttaatctgaattgaggatgttaactggtaggtgaggccaactttaaccctactactgtcgttttacaaggttattaaaatcgatttgatgttattaaatttgaaatagtcgccatttaaaaataaaaaaatatagaagacaattaaattggattggtttttagattcaaatttaaaaattaatctaaactaaactaaaattttataataacatgtcttataatgtattaagtttttattattgttattgagtttggaagcatataataaggaacataagttaatatttatgatccaataagatcgacttagtattagaatatttagatgatttgtatgagatattcaaacttcattcttatcatagagagacaaaagttaatattgattgtggagcaaaaaatgttattgggagcatatgcaaagataagatggataaaatatgctgcagcctgcagagcaagtgttttgtcaagtgacaactattcatgatgtgaacacaagaatcctttctatttatctatcccacataaaatacttattttaataatggtggtaaataatgattaaaatatttgattattggttaaaataaataaggaaaatataaacattctcatggaaagtttttgttaaagaatttgccaaatattgattaaaatatttgattcttggttaggaaaaatcagcaatgcctaagaatatttttttccaaaccttgtagagtaccaaacacaatctaagaatttttcaaatctatacgtgttcagaaactt

SEQ ID NO: 11 - DNA, 1164 bp, Glycine max
atgggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattga

SEQ ID NO: 12 - DNA, 1164 bp, Glycine max
atgggtctagcaaaggaaacaataatgggaggtggaggccgtgtggccaaagttgaaattcagcagaagaagcctctctcaagggttccaaacacaaagccaccattcactgttggccaactcaagaaagccattccaccgcactgctttcagcgttccctcctcacttcattgtcctatgttgtttatgacctttcattggctttcattttctacattgccaccacctacttccacctcctccctcaccccttttccctcattgcatggccaatctattgggttctccaaggttgcattcttactggcgtgtgggtgattgctcacgagtgtggtcaccatgccttcagcaagtacccatgggttgatgatgttatgggtttgaccgttcactcagcacttttagtcccttatttctcatggaaaataagccatcgccgccaccactccaacacgggttcccttgaccgtgatgaagtgtttgtcccaaaaccaaaatccaaagttgcatggtacaccaagtacctgaacaaccctctaggaagggctgcttctcttctcatcacactcacaatagggtggcctttgtatttagccttcaatgtctctggcagaccctatgatggttttgctagccactaccacccttatgctcccatatattcaaatcgtgagaggcttttgatctatgtctctgatgttgctttgttttctgtgacttacttgctctaccgtgttgcaactatgaaagggttggtttggctgctatgtgtttatggggtgccattgctcattgtgaacggttttcttgtgaccatcacatatctgcagcacacacactatgccttgcctcactatgattcatcagaatgggattggctgaggggtgctttggcaactatggacagagattatggaattctgaacaaggtgtttcaccacataactgatactcatgtggctcaccatcttttctctacaatgccacattaccatgcaacggaggcaaccaatgcaatgaagccaatattgggtgagtactaccgatttgatgacacaccattttacaaggcactgtggagagaagcaagagagtgcctctatgtggagccagatgaaggaacatccgagaagggcgtgtattggtacaggaacaagtattga

SEQ ID NO: 13 - DNA, 4014 bp, Artificial Sequence
  source: /note=“Description of Artificial Sequence Synthetic polynucleotide”
ttctaactttattataattctattattgactgacttaagcgtcagagtacctttacatgtaccactcccaccacccgaaaaagcttagaataccaagtagaagattagattattggtggagatcgatttgaagagcacttcagtggtaagaacatttcctaaattacatttctaatttgtcacaaaaatttcatactatagatgcaagtattgtgctaaaatgatgtaaggcccacggaactcaaatgtttccttggctgttgcagattccccggccctagagactttgacgtttgatccacaccacatgtcatctcctttcagaagtattggactcagagcagacttaatattattgtggccacctgagctaaatactggtgtgctctaattaactctatgatgtgctctgctttcagcagcagccaatgcagtctggccaagtatgatgactaagctcaaatcctttcccagtgctactaatttctttgtcctctgaactaataccaataaggatggaatgtgcatatatatacatagatagcaggtcaacacaaacaatatagcatattaccataattccataaattaattggacaattttcaatatctcaattcaattgaaaacctcttgcacaaggtgaacagataccattgcgaagcacatgtaatatagtaaatgtctaatgaactagaagaatgcatttatagttaagtgtagacattagacagcattaacaaatatattgtcagggagcagaagatcattacctttctgatttcatccaagaggttataaaactgagcattgtggggaccatgttgttcattatggcaaaggtcgttcagcatttatcaaaaatctgctcataagggaagaaatctcactcgcgcgcggttagggctccatagtcttatctctatttctgcacctggtccaatatttggctttttcaccaaaaacaagttaaaaacgaaaactaattattataatatgttgatcaagaaaacatttattaaaataaattatttttgttactgtagaaatataagaggcacctggtctaatattcattttgtgttggcttgatggattatattaaaatgatatatcctgttacaaaaatcatctattttaataaatatttgtttttccaactcattatacttttttcgttttttaatgatgaaaaggctatcactatttatccctaaaagtgatggattcgcaggccaggactgcactttaaagttctagagaactaatactagtacaggtgcataacattaaaacataaagtgttcatcatgttagagaataccacccactagggagtcctaaggccttagaaggactcaagagaaggtgtggtaaagagtgtatctttttataattaattaaaaatagttattaattttttattatatattatgtacttacgtatttttattaaaaatttaatatttttataatttattatatatacttacgtatttttggtataattctatttattaatttttaattaacaatttttttactctttatctattaaagtaataaagaatatagaacatatgtgtgataatcaaaacgtagtaattttaatttttatttataatttttaattgacaattataactttatttaacaattataacaaattattaaaagaagttttaaagtattataatatttgtttatgttggaaaatgaataaaataaaataaaaaaaaggatgtgataaaggaagatatataatatttaaaattaacaagcttatctccattacacatttaaaataatatatttgtaaaacaagagaaagcacactaaaccaggggcgaataaatttctctccctttgttcctgctcctggttggctttacattagcatttatagccaagccaagctagatcaaagacaaagtgtgttgcttaacgttaacatgttccactaaaacagaaaaattaagagagaaagctgaaaattaatttgatgcctccagaaaagaaagaaattttcaagtccttggagggatgggcctcggagtgggtcctaccgctgctgaagcccgtggagcaatgctggcagccacaaaacttcctccctgacccctcccttccgcatgaagagttcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgagcccgagcccgtgggccgtgtggacccgggcctggaccgccgaggaaaacagacacggggatctgctcagaacttatttgtatctctctgggagggttgacatggctaaggtcgaaaagaccgtacattacctcatttcagctggcatggaccctgggacagacaacaacccatatttggggtttgtgtacacgtcattccaagagcgagcaacatttgtggcgcacgggaacacggctcggctcgcgaaggagggcggggatccagtgctggcgcgcctatgcgggaccatcgcagcggacgagaagcggcacgagaacgcgtactcaagaatcgtggagaagcttctggaagtggaccccaccggggcaatggtggccatagggaacatgatggagaagaagatcacgatgccggcgcaccttatgtacgatggggatgaccccaggctattcgagcactactccgctgtggcgcagcgcataggcgtgtacaccgccaacgactacgcagacatcttggagtttctcgttgaacggtggagattggagaagcttgaaggattgatggctgaggggaagcgggcgcaggatttcgtgtgtgggttggcgccgaggattaggaggttgcaagagcgcgctgatgagcgagcgcgtaagatgaagaagcatcatggcgttaagttcagttggattttcaataaagaattgcttttgtgactagcctttcaaccatcattatatgccgtacgtacgactcaaattaaataaataaacggctagctcaaggtttctatttgcttaataaattcttgtcttttgttattattctcgttgcactcgcacagttacttccctgcttattgttttaggaatacaatataattctatatatttgagtttgagttggtgcatacgacaaatcgacaatgccaatttctttttatacaataaattcttgtatttttcttttataattacgattaggatgttttctcactagagagctttgtataatgtatcagatttgcggatctactattaggatcgatgtttacccaataaagcattatatcatgtatgcataaatagttttgtaaaaaaaaatgtatattgatcgttggtaatgtcactaactttcgcttgtaactgttgttggtaaattggtaatgtcgtcacttacgaattggatctattaggaggttaaatacgggtcaccttgtgacagagttgattgttctcagatccagaactactaatttgttggctaaccttaatatcttatttattcattccaatcttttccattatctatatattctccaagatctttttagttaaagttgtaaaatcaattttcctgaaaaacaaaaaaaaaggctaataatacactttttaatatattatttctaacacattttattattagttaaaatttattaataactataaaattaagggaaaaaaccattaaataatatattaaaaagtatgtttgaaatcccaaacgtgtgaaagtagacttgctaatgaggtaaacttgctatttacatttttcttacgagaggagattcaaataataattgtttaggggtgttgcatgtaattgtaaaggggttgtgtctcggaagggtttttattctttgttgtcatgcaggtttggacactacttgtcttagagctgtatacctttcaaaaaataaataaattcattttttct

SEQ ID NO: 14 - DNA, 4251 bp, Artificial Sequence
  source: /note=“Description of Artificial Sequence Synthetic polynucleotide”
tgcgtcaacatttatataatatatagaaaaaaatttgaaattaatcacaaaaactaaaattaagaatttgtctaaaataagaataaagtatctcaattaaaaaataaaaactaaaatcacaaattttaaaaaagtgaaggataaaatgtatcatttaaaaaatgggaaaacgaaaatcacatatttaaaaaaataagagatagaaattgcattttaatatttttttttatttctcttccttttttaattatacttttaatcacattaatgattttattttctatttctcttctttccacctacatacatcccaaagatggagggtgcaattgtaagtttattagcactcttgtttttacctgcatttgtgtgtgctaaccaaattgcattcttctctttacataatgtatttgatttgaattttcataccacatgcaagcatgattacgtacgtgtccatgatcaaatacaaatgctgtctggtactggcaatttggtaaacagccatccatttttttttgtctctaattattctctagaatatctgaagattcctctgtcatcgaattccttgcttggtaacaacgtcgtcaagttattattttgttctttttttttttatcatatttcttattttgttccaagtatgtcatattttgatccatcttgacaagtagattgtcatgtaggaataggaatatcactttaaattttaaagcattgattagtctgtaggcaatattgtcttcttcttcctccttattaatattttttattctgccttcaatcaccagttatgggagatggatgtaatactaaataccatagttgttctgcttgaagtttagttgtatagttgttctgcttgaagtttagttgtgtgtaatgtttcagcgttggcttcccctgtaactgctacaatggtactgaatatatattttttgcattgttcatttttttcttttacttaatcttcattgctttgaaattaataaaacaaaaagaaggaccgaatagtttgaagtttgaactattgcctattcatgtaacttattcacccaatcttatatagtttttctggtagagatcattttaaattgaaggatataaattaagaggaaatacttgtatgtgatgtgtggcaatttggaagatcatgcgtagagagtttaatggcaggttttgcaaattgacctgtagtcataattacactgggccctctcggagttttgtgcctttttgttgtcgctgtgtttggttctgcatgttagcctcacacagatatttagtagttgttgttctgcatataagcctcacacgtatactaaacgagtgaacctcaaaatcatggccttacacctattgagtgaaattaatgaacagtgcatgtgagtatgtgactgtgacacaacccccggttttcatattgcaatgtgctactgtggtgattaaccttgctacactgtcgtccttgtttgtttccttatgtatattgataccataaattattactagtatatcattttatattgtccataccattacgtgtttatagtctctttatgacatgtaattgaattttttaattataaaaaataataaaacttaattacgtactataaagagatgctcttgactagaattgtgatctcctagtttcctaaccatatactaatatttgcttgtattgatagcccctccgttcccaagagtataaaactgcatcgaataatacaagccactaggcatggtaaattaaattgtgcctgcacctcgggatatttcatgtggggttcatcatatttgttgaggaaaagaaactcccgaaattgaattatgcatttatatatcctttttcatttctagatttcctgaaggcttaggtgtaggcacctagctagtagctacaatatcagcacttctctctattgataaacaattggctgtaatgccgcagtagaggacgatcacaacatttcgtgctggttactttttgttttatggtggcaacagctgctacttcatcatttttccctgttacttcaccctcgccggactctggtggagcaggcagcaaacttggtggtgggcctgcaaaccttggaggactaaaatccaaatctgcgtcttctggtggcttgaaggcaaaggcgcaagccccttcgaaaattaatggaaccacagttgttacatctaaagaaagcttcaagcatgatgatgatctaccttcgcctccccccagaacttttatcaaccagttgcctgattggagcatgcttcttgctgctatcacaacaattttcttggccgctgaaaagcagtggatgatgcttgattggaagccacggcgacctgacatgcttattgacccctttgggataggaaaaattgttcaggatggtcttgtgttccgtgaaaacttttctattagatcatatgagattggtgctgatcgtaccgcatctatagaaacagtaatgaaccatttgcaagaaactgcacttaatcatgttaaaagtgctgggcttcttggtgatggctttggttccacgccagaaatgtgcaaaaagaacttgatatgggtggttactcggatgcaggttgtggtggaacgctatcctacatggggtgacatagttcaagtggacacttgggtttctggatcagggaagaatggtatgcgccgtgattggcttttacgtgactgcaaaactggtgaaatcttgacaagagcttccagtgtttgggtcatgatgaataagctaacacggaggctgtctaaaattccagaagaagtcagacaggagataggatcttattttgtggattctgatccaattctggaagaggataacagaaaactgactaaacttgacgacaacacagcggattatattcgtaccggtttaagtcctaggtggagtgatctagatatcaatcagcatgtcaacaatgtgaagtacattggctggattctggagagtgctccacagccaatcttggagagtcatgagctttcttccatgactttagagtataggagagagtgtggtagggacagtgtgctggattccctgactgctgtatctggggccgacatgggcaatctagctcacagcgggcatgttgagtgcaagcatttgcttcgactggaaaatggtgctgagattgtgaggggcaggactgagtggaggcccaaacctgtgaacaactttggtgttgtgaaccaggttccagcagaaagcacctaatggagcaaccaatgggccatagtgggagttatggaagttttgtcatgtattagtacataattagtagaatgttataaataagtggatttgccgcgtaatgactttgtgtgtattgtgaaacagcttgttgcgatcatggttataatgtaaaaataattctggtattaattacatgtggaaagtgttctgcttatagctttctgcctaaaatgcacgctgcacgggacaatatcattggtaatttttttaaaatctgaattgaggctactcataatactatccataggacatcaaagacatgttgcattgactttaagcagaggttcatctagaggattactgcataggcttgaactacaagtaatttaagggacgagagcaactttagctctaccacgtcgttttacaaggttattaaaatcaaattgatcttattaaaactgaaaatttgtaataaaatgctattgaaaaattaaaatatagcaaacacctaaattggactgatttttagattcaaatttaataattaatctaaattaaacttaaattttataatatatgtcttgtaatatatcaagtttttttttttattattgagtttggaaacatataataaggaacattagttaatattgataatccactaagatcgacttagtattacagtatttggatgatttgtatgagatattcaaacttcactcttatcataatagagacaaaagttaatactgatggtggagaaaaaaaaatgttattgggagcatatggtaagataagacggataaaaatatgctgcagcctggagagctaatgtattttttggtgaagttttcaagtgacaactattcatgatgagaacacaataatattttctacttacctatcccacataaaatactgattttaataatgatgataaataatgattaaaatatttgattctttgttaagagaaataaggaaaacataaatattctcatggaaaaatcagcttgta

SEQ ID NO: 15 - DNA, 49 bp, Glycine max
tggagggatgggcctcggagtgggtcctaccgctgctgaagcccgtgga

SEQ ID NO: 16 - DNA, 17 bp, Glycine max
tggagggatgggcctcg

SEQ ID NO: 17 - DNA, 17 bp, Glycine max
tccacgggcttcagcag

SEQ ID NO: 18 - DNA, 49 bp, Glycine max
tgggtcctaccgctgctgaagcccgtggagcaatgctggcagccacaaa

SEQ ID NO: 19 - DNA, 17 bp, Glycine max
tgggtcctaccgctgct

SEQ ID NO: 20 - DNA, 17 bp, Glycine max
tttgtggctgccagcat

SEQ ID NO: 21 - DNA, 49 bp, Glycine max
tcctccctgacccctcccttccgcatgaagagttcagccatcaggtgaa

SEQ ID NO: 22 - DNA, 17 bp, Glycine max
tcctccctgacccctcc

SEQ ID NO: 23 - DNA, 17 bp, Glycine max
ttcacctgatggctgaa

SEQ ID NO: 24 - DNA, 49 bp, Glycine max
tccctgacccctcccttccgcatgaagagttcagccatcaggtgaagga

SEQ ID NO: 25 - DNA, 17 bp, Glycine max
tccctgacccctccctt

SEQ ID NO: 26 - DNA, 17 bp, Glycine max
tccttcacctgatggct

SEQ ID NO: 27 - DNA, 49 bp, Glycine max
tacctgatgagtactttgtggtgctggtgggtgatatggtcaccgagga

SEQ ID NO: 28 - DNA, 17 bp, Glycine max
tacctgatgagtacttt

SEQ ID NO: 29 - DNA, 17 bp, Glycine max
tcctcggtgaccatatc

SEQ ID NO: 30 - DNA, 49 bp, Glycine max
tccgcggcgccgttcaaagcccggaaggcccactcaatgcctccagaaa

SEQ ID NO: 31 - DNA, 17 bp, Glycine max
tccgcggcgccgttcaa

SEQ ID NO: 32 - DNA, 17 bp, Glycine max
tttctggaggcattgag

SEQ ID NO: 33 - DNA, 49 bp, Glycine max
taaattatcaacaaaccaagggctaatcactagtcacaccctttacaaa

SEQ ID NO: 34 - DNA, 17 bp, Glycine max
taaattatcaacaaacc

SEQ ID NO: 35 - DNA, 17 bp, Glycine max
tttgtaaagggtgtgac

SEQ ID NO: 36 - DNA, 49 bp, Glycine max
tagtcacaccctttacaaatatctccaacctctccacagttccactcaa

SEQ ID NO: 37 - DNA, 17 bp, Glycine max
tagtcacaccctttaca

SEQ ID NO: 38 - DNA, 17 bp, Glycine max
ttgagtggaactgtgga

SEQ ID NO: 39 - DNA, 49 bp, Glycine max
tcaagtacaatagacacgtaatcaaaaccatgcagatacgaacctgcca

SEQ ID NO: 40 - DNA, 17 bp, Glycine max
tcaagtacaatagacac

SEQ ID NO: 41 - DNA, 17 bp, Glycine max
tggcaggttcgtatctg

SEQ ID NO: 42 - DNA, 49 bp, Glycine max
tcaccacccaaacccttccacaacttccgtgttcttctagaaaagccca

SEQ ID NO: 43 - DNA, 17 bp, Glycine max
tcaccacccaaaccctt

SEQ ID NO: 44 - DNA, 17 bp, Glycine max
tgggcttttctagaaga

SEQ ID NO: 45 - DNA, 49 bp, Glycine max
ttccgccgttaaacgctgcggtttccgcggcgccgttcaaagcccggaa

SEQ ID NO: 46 - DNA, 17 bp, Glycine max
ttccgccgttaaacgct

SEQ ID NO: 47 - DNA, 17 bp, Glycine max
ttccgggctttgaacgg

SEQ ID NO: 48 - DNA, 49 bp, Glycine max
ttagtccgattgatttctcgatatcatttaaggctaaggttgacctcta

SEQ ID NO: 49 - DNA, 17 bp, Glycine max
ttagtccgattgatttc

SEQ ID NO: 50 - DNA, 17 bp, Glycine max
tagaggtcaaccttagc

SEQ ID NO: 51 - DNA, 49 bp, Glycine max
tcttctaacttgcgtatattttgcatgcagcgaccttagaaattcatta

SEQ ID NO: 52 - DNA, 17 bp, Glycine max
tcttctaacttgcgtat

SEQ ID NO: 53 - DNA, 17 bp, Glycine max
taatgaatttctaaggt

SEQ ID NO: 54 - DNA, 49 bp, Glycine max
tttgcatttctcttctttatcccctttctgtggaaggtgggagggaaaa

SEQ ID NO: 55 - DNA, 17 bp, Glycine max
tttgcatttctcttctt

SEQ ID NO: 56 - DNA, 17 bp, Glycine max
ttttccctcccaccttc

SEQ ID NO: 57 - DNA, 49 bp, Glycine max
tgtgatataactgatgtgctgtgctgttattatttgttatttggggtga

SEQ ID NO: 58 - DNA, 17 bp, Glycine max
tgtgatataactgatgt

SEQ ID NO: 59 - DNA, 17 bp, Glycine max
tcaccccaaataacaaa

SEQ ID NO: 60 - DNA, 49 bp, Glycine max
ttattatttgttatttggggtgaagtataattttttgggtgaacttgga

SEQ ID NO: 61 - DNA, 17 bp, Glycine max
ttattatttgttatttg

SEQ ID NO: 62 - DNA, 17 bp, Glycine max
tccaagttcacccaaaa

SEQ ID NO: 63 - DNA, 297 bp, Glycine max
caatgcctccagaaaagaaagaaattttcaagtcctggagggatgggcctcggagtgggtcctaccgctgctgaagcccgtggagcaatgctggcagccacaaaacttcctccctgacccctcccttccgcatgaagagttcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgag

SEQ ID NO: 64 - DNA, 272 bp, Artificial Sequence
  source: /note=“Description of Artificial Sequence Synthetic polynucleotide”
caatgcctccagaaaagaaagaaattttcaagtccttggagggatgggcctcggagtgggtcctaccgctgctgaagcccgtggagcaatgctggcagccacaaaacttcctccctcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgag

SEQ ID NO: 65 - DNA, 284 bp, Artificial Sequence
  source: /note=“Description of Artificial Sequence Synthetic polynucleotide”
caatgcctccagaaaagaaagaaattttcaagtccttggagggatgggcctcggagtgggtcctaccgctgctgaagcccgtggagcaatgctggcagccacaaaacttcctccctgacccctcccttccgccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgag

SEQ ID NO: 66 - DNA, 246 bp, Artificial Sequence
  source: /note=“Description of Artificial Sequence Synthetic polynucleotide”
caatgcctccagaaaagaaagaaattttcaagtccttggagggatgggcctcggagtgggtcctaccgctgctgaagcccgtgaagagttcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgag

SEQ ID NO: 67 - DNA, 293 bp, Artificial Sequence
  source: /note=“Description of Artificial Sequence Synthetic polynucleotide”
caatgcctccagaaaagaaagaaattttcaagtccttggagggatgggcctcggagtgggtcctaccgctgctgaagcccgtggagcaatgctggcagccacaaaacttcctccctgacccctcccttcgaagagttcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgag

SEQ ID NO: 68 - DNA, 294 bp, Artificial Sequence
  source: /note=“Description of Artificial Sequence Synthetic polynucleotide”
caatgcctccagaaaagaaagaaattttcaagtccttggagggatgggcctcggagtgggtcctaccgctgctgaagcccgtggagcaatgctggcagccacaaaacttcctccctgacccctcccttcgtcagagttcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgag

SEQ ID NO: 69 - DNA, 290 bp, Artificial Sequence
  source: /note=“Description of Artificial Sequence Synthetic polynucleotide”
caatgcctccagaaaagaaagaaattttcaagtccttggagggatgggcctcggagtgggtcctaccgctgctgaagcccgtggagcaatgctggcagccacaaaacttcctccctgacccctccctaagagttcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgag

SEQ ID NO: 70 - DNA, 284 bp, Artificial Sequence
  source: /note=“Description of Artificial Sequence Synthetic polynucleotide”
caatgcctccagaaaagaaagaaattttcaagtccttggagggatgggcctcggagtgggtcctaccgctgctgaagcccgtggagcaatgctggcagccacaaaacttcctccctgacccctcccttcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgag

SEQ ID NO: 71 - DNA, 100 bp, Glycine max
accgctgctgaagcccgtggagcaatgctggcagccacaaaacttcctccctgacccctcccttccgcatgaagagttcagccatcaggtgaaggagctt

SEQ ID NO: 72 - DNA, 74 bp, Artificial Sequence
  source: /note=“Description of Artificial Sequence Synthetic oligonucleotide”
accgctgctgaagcccgtggagcaatgctggcagccacaaaacttcctccctcagccatcaggtgaaggagctt

SEQ ID NO: 73 - DNA, 86 bp, Artificial Sequence
  source: /note=“Description of Artificial Sequence Synthetic oligonucleotide”
accgctgctgaagcccgtggagcaatgctggcagccacaaaacttcctccctgacccctcccttccgccatcaggtgaaggagctt

SEQ ID NO: 74 - DNA, 48 bp, Artificial Sequence
  source: /note=“Description of Artificial Sequence Synthetic oligonucleotide”
accgctgctgaagcccgtgaagagttcagccatcaggtgaaggagctt

SEQ ID NO: 75 - DNA, 95 bp, Artificial Sequence
  source: /note=“Description of Artificial Sequence Synthetic oligonucleotide”
accgctgctgaagcccgtggagcaatgctggcagccacaaaacttcctccctgacccctcccttcgaagagttcagccatcaggtgaaggagctt

SEQ ID NO: 76 - DNA, 96 bp, Artificial Sequence
  source: /note=“Description of Artificial Sequence Synthetic oligonucleotide”
accgctgctgaagcccgtggagcaatgctggcagccacaaaacttcctccctgacccctcccttcgtcagagttcagccatcaggtgaaggagctt

SEQ ID NO: 77 - DNA, 92 bp, Artificial Sequence
  source: /note=“Description of Artificial Sequence Synthetic oligonucleotide”
accgctgctgaagcccgtggagcaatgctggcagccacaaaacttcctccctgacccctccctaagagttcagccatcaggtgaaggagctt

SEQ ID NO: 78 - DNA, 86 bp, Artificial Sequence
  source: /note=“Description of Artificial Sequence Synthetic oligonucleotide”
accgctgctgaagcccgtggagcaatgctggcagccacaaaacttcctccctgacccctcccttcagccatcaggtgaaggagctt

SEQ ID NO: 79 - DNA, 3500 bp, Artificial Sequence
  source: /note=“Description of Artificial Sequence Synthetic polynucleotide”
atacgtatataatatttaattatatatactttgatcaatatgtttagtgatatttaaaaataattctacaaataattaattagcaagtatttataaaatccctcaaagaatctatacccaaaaacagctagcaaaaataatcatgcatcgagttttaaaaacataataactctaataaataattaatcaaataaatttaatttcctagtcgagcttgtaatttgtatggttaaatcatattcaaatagcaatgattttatatattttcatattaaattttcatttttatgtgaagtgaaaatttaaatttaagatatatgtacgaataaagctaacaaataaaatattaaattatacaagttaatttcttttacagtaaattatattcaattcaatcatctttttcatcatatgattgaagatatatatttatcttaagtaaatactattttatttccatatcttttgtttcatactttaaaatagaagcttccttattgttattatttcttgtaagttattagacccaaaatctttcatatacacaaaattatctttaaattaatataaaaatattaataacatatatttcataaaatatcaaaatttatatccctgaaaaaaattgtagtgatgttttcttttagagaaaaaatgattatgaacactgcactatcatatcataatccactgttaacttttaaaattatcttaaaataattttgtttataaatgacaatataaaattatttctaactttattataattctattattgactgacttaagcgtcagagtacctttacatgtaccactcccaccacccgaaaaagcttagaataccaagtagaagattagattattggtggagatcgatttgaagagcacttcagtggtaagaacatttcctaaattacatttctaatttgtcacaaaaatttcatactatagatgcaagtattgtgctaaaatgatgtaaggcccacggaactcaaatgtttccttggctgttgcagattccccggccctagagactttgacgtttgatccacaccacatgtcatctcctttcagaagtattggactcagagcagacttaatattattgtggccacctgagctaaatactggtgtgctctaattaactctatgatgtgctctgctttcagcagcagccaatgcagtctggccaagtatgatgactaagctcaaatcctttcccagtgctactaatttctttgtcctctgaactaataccaataaggatggaatgtgcatatatatacatagatagcaggtcaacacaaacaatatagcatattaccataattccataaattaattggacaattttcaatatctcaattcaattgaaaacctcttgcacaaggtgaacagataccattgcgaagcacatgtaatatagtaaatgtctaatgaactagaagaatgcatttatagttaagtgtagacattagacagcattaacaaatatattgtcagggagcagaagatcattacctttctgatttcatccaagaggttataaaactgagcattgtggggaccatgttgttcattatggcaaaggtcgttcagcatttatcaaaaatctgctcataagggaagaaatctcactcgcgcgcggttagggctccatagtcttatctctatttctgcacctggtccaatatttggctttttcaccaaaaacaagttaaaaacgaaaactaattattataatatgttgatcaagaaaacatttattaaaataaattatttttgttactgtagaaatataagaggcacctggtctaatattcattttgtgttggcttgatggattatattaaaatgatatatcctgttacaaaaatcatctattttaataaatatttgtttttccaactcattatacttttttcgttttttaatgatgaaaaggctatcactatttatccctaaaagtgatggattcgcaggccaggactgcactttaaagttctagagaactaatactagtacaggtgcataacattaaaacataaagtgttcatcatgttagagaataccacccactagggagtcctaaggccttagaaggactcaagagaaggtgtggtaaagagtgtatctttttataattaattaaaaatagttattaattttttattatatattatgtacttacgtatttttattaaaaatttaatatttttataatttattatatatacttacgtatttttggtataattctatttattaatttttaattaacaatttttttactctttatctattaaagtaataaagaatatagaacatatgtgtgataatcaaaacgtagtaattttaatttttatttataatttttaattgacaattataactttatttaacaattataacaaattattaaaagaagttttaaagtattataatatttgtttatgttggaaaatgaataaaataaaataaaaaaaaggatgtgataaaggaagatatataatatttaaaattaacaagcttatctccattacacatttaaaataatatatttgtaaaacaagagaaagcacactaaaccaggggcgaataaatttctctccctttgttcctgctcctggttggctttacattagcatttatagccaagccaagctagatcaaagacaaagtgtgttgcttaacgttaacatgttccactaaaacagaaaaattaagagagaaagctgaaaattaatttgatgcctccagaaaagaaagaaattttcaagtccttggagggatgggcctcggagtgggtcctaccgctgctgaagcccgtggagcaatgctggcagccacaaaacttcctccctgacccctcccttccgcatgaagagttcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgagcccgagcccgtgggccgtgtggacccgggcctggaccgccgaggaaaacagacacggggatctgctcagaacttatttgtatctctctgggagggttgacatggctaaggtcgaaaagaccgtacattacctcatttcagctggcatggtaagtgtttcactgttttttattttttatttataatttgaattcggttgactagattctattgattgggtgagtatgcatttcttttttacttgacatatggcaggagtaatttcttttgtatatacgtgtttaaaattggatattgttcgtctagcgatttttgcttctcttcattttaatttctgaaggtttcgagatgtaccattccacatgaagcaaaaattaattgaaatttataatttaaaagaggtgatattctatgtactattaaattgaacaaaattgttattcataacaatatat

SEQ ID NO: 80 - DNA, 1449 bp, Artificial Sequence
  source: /note=“Description of Artificial Sequence Synthetic polynucleotide”
cattacacatttaaaataatatatttgtaaaacaagagaaagcacactaaaccaggggcgaataaatttctctccctttgttcctgctcctggttggctttacattagcatttatagccaagccaagctagatcaaagacaaagtgtgttgcttaacgttaacatgttccactaaaacagaaaaattaagagagaaagctgaaaattaatttgatgcctccagaaaagaaagaaattttcaagtccttggagggatgggcctcggagtgggtcctaccgctgctgaagcccgtggagcaatgctggcagccacaaaacttcctccctgacccctcccttccgcatgaagagttcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgagcccgagcccgtgggccgtgtggacccgggcctggaccgccgaggaaaacagacacggggatctgctcagaacttatttgtatctctctgggagggttgacatggctaaggtcgaaaagaccgtacattacctcatttcagctggcatggtaagtgtttcactgttttttattttttatttataatttgaattcggttgactagattctattgattgggtgagtatgcatttcttttttacttgacatatggcaggagtaatttcttttgtatatacgtgtttaaaattggatattgttcgtctagcgatttttgcttctcttcattttaatttctgaaggtttcgagatgtaccattccacatgaagcaaaaattaattgaaatttataatttaaaagaggtgatattctatgtactattaaattgaacaaaattgttattcataacaatatataaaagaaaatatttccttttttgatctctacatttgtcgattattttgtcttttaattgtttttaaaactactcactactatattctcttccattttttatttaacatctactctctattcttctttcatttttttttgttttataaaaagtacagagactcttcaattttctatttaacgtttactctttatttctctttctttttatattttaacaagaaaaatagagttctgtatctctctttaatttatatataagctctgtagcatgccggtccacatcacatataatagtttaaaattaaaattaattaatgtacttaacagctgcatagctcatattggtatattaaattgtacaagaaaaactgaaaagccaaacaaacatttttagtatcaataaattcttggtttcacgtgacatggcaatcggagctttctcatagcaagttgggcagacttaaattgaacagttaagtagattt

SEQ ID NO: 81 - DNA, 3500 bp, Artificial Sequence
  source: /note=“Description of Artificial Sequence Synthetic polynucleotide”
ctgttgttacttttcatactatatttatatcaactatttgcttaacaacaggaaactgcacttaatcatgttaaaagtgctgggcttcttggtgatggctttggttccacgccagaaatgtgcaaaaagaacttgatatctatgaaaatattcgaaattcattcatgtgaaatttttagtatattttttatttacataaaattaaaatttattattttttacctagatataatcttgagtaaatttaaacttatggtgatgattttatatctatgaaaaatgtgattttttttatattggtggttacaaccaatataaaaaataatacaaataagttcaaatatttaaatcttactggggttaaaaattgacacgtttcaattttataaacatgaaaaatattcttgaaaattttaaaaaacaaattttgaatatttttatatttatgataattaaaaatatattttaatctaataaaaatgcattaaaaaagaatagtagctaatatataaaattaaaattctaaagtagaaaaaaaagattaaaccttatttttatcgagtaaacttgatagagataaataatgatacgtggtggtggggtgattacaaaatgtcttaatcttttattgtgaaagaaatattctattgtgaataaaaaaaaaaacccaatgtctttatcttttacttggaaaatgaaaaaaaaaaaaaaaactctgtaaaatcccgtgggtactggcattactaggagagtatggccgaaataaggggggcaatacggtaaaagagggaagagactagctgggatctttgaaagggcggcgggagggccagctggacagtataaaaagaagtggctggaatgctaatgccttatccctaactcataatgcgtcaacatttatataatatatagaaaaaaatttgaaattaatcacaaaaactaaaattaagaatttgtctaaaataagaataaagtatctcaattaaaaaataaaaactaaaatcacaaattttaaaaaagtgaaggataaaatgtatcatttaaaaaatgggaaaacgaaaatcacatatttaaaaaaataagagatagaaattgcattttaatatttttttttatttctcttccttttttaattatacttttaatcacattaatgattttattttctatttctcttctttccacctacatacatcccaaagatggagggtgcaattgtaagtttattagcactcttgtttttacctgcatttgtgtgtgctaaccaaattgcattcttctctttacataatgtatttgatttgaattttcataccacatgcaagcatgattacgtacgtgtccatgatcaaatacaaatgctgtctggtactggcaatttggtaaacagccatccatttttttttgtctctaattattctctagaatatctgaagattcctctgtcatcgaattccttgcttggtaacaacgtcgtcaagttattattttgttctttttttttttatcatatttcttattttgttccaagtatgtcatattttgatccatcttgacaagtagattgtcatgtaggaataggaatatcactttaaattttaaagcattgattagtctgtaggcaatattgtcttcttcttcctccttattaatattttttattctgccttcaatcaccagttatgggagatggatgtaatactaaataccatagttgttctgcttgaagtttagttgtatagttgttctgcttgaagtttagttgtgtgtaatgtttcagcgttggcttcccctgtaactgctacaatggtactgaatatatattttttgcattgttcatttttttcttttacttaatcttcattgctttgaaattaataaaacaaaaagaaggaccgaatagtttgaagtttgaactattgcctattcatgtaacttattcacccaatcttatatagtttttctggtagagatcattttaaattgaaggatataaattaagaggaaatacttgtatgtgatgtgtggcaatttggaagatcatgcgtagagagtttaatggcaggttttgcaaattgacctgtagtcataattacactgggccctctcggagttttgtgcctttttgttgtcgctgtgtttggttctgcatgttagcctcacacagatatttagtagttgttgttctgcatataagcctcacacgtatactaaacgagtgaacctcaaaatcatggccttacacctattgagtgaaattaatgaacagtgcatgtgagtatgtgactgtgacacaacccccggttttcatattgcaatgtgctactgtggtgattaaccttgctacactgtcgtccttgtttgtttccttatgtatattgataccataaattattactagtatatcattttatattgtccataccattacgtgtttatagtctctttatgacatgtaattgaattttttaattataaaaaataataaaacttaattacgtactataaagagatgctcttgactagaattgtgatctcctagtttcctaaccatatactaatatttgcttgtattgatagcccctccgttcccaagagtataaaactgcatcgaataatacaagccactaggcatggtaaattaaattgtgcctgcacctcgggatatttcatgtggggttcatcatatttgttgaggaaaagaaactcccgaaattgaattatgcatttatatatcctttttcatttctagatttcctgaaggcttaggtgtaggcacctagctagtagctacaatatcagcacttctctctattgataaacaattggctgtaatgccgcagtagaggacgatcacaacatttcgtgctggttactttttgttttatggtggcaacagctgctacttcatcatttttccctgttacttcaccctcgccggactctggtggagcaggcagcaaacttggtggtgggcctgcaaaccttggaggactaaaatccaaatctgcgtcttctggtggcttgaaggcaaaggcgcaagccccttcgaaaattaatggaaccacagttgttacatctaaagaaagcttcaagcatgatgatgatctaccttcgcctccccccagaacttttatcaaccagttgcctgattggagcatgcttcttgctgctatcacaacaattttcttggccgctgaaaagcagtggatgatgcttgattggaagccacggcgacctgacatgcttattgacccctttgggataggaaaaattgttcaggatggtcttgtgttccgtgaaaacttttctattagatcatatgagattggtgctgatcgtaccgcatctatagaaacagtaatgaaccatttgcaagtaagtccgtcctcatacaagtgaatctttatgatcttcagagatgagtatgctttgactaagatagggctgtttatttagtcactgtaattcaatttcatatatagataatatcattg

SEQ ID NO: 82 - DNA, 8200 bp, Artificial Sequence
  source: /note=“Description of Artificial Sequence Synthetic polynucleotide”
agaaaaaatgaatttatttattttttgaaaggtatacagctctaagacaagtagtgtccaaacctgcatgacaacaaagaataaaaacccttccgagacacaacccctttacaattacatgcaacacccctaaacaattattatttgaatctcctctcgtaagaaaaatgtaaatagcaagtttacctcattagcaagtctactttcacacgtttgggatttcaaacatactttttaatatattatttaatggttttttcccttaattttatagttattaataaattttaactaataataaaatgtgttagaaataatatattaaaaagtgtattattagcctttttttttgtttttcaggaaaattgattttacaactttaactaaaaagatcttggagaatatatagataatggaaaagattggaatgaataaataagatattaaggttagccaacaaattagtagttctggatctgagaacaatcaactctgtcacaaggtgacccgtatttaacctcctaatagatccaattcgtaagtgacgacattaccaatttaccaacaacagttacaagcgaaagttagtgacattaccaacgatcaatatacatttttttttacaaaactatttatgcatacatgatataatgctttattgggtaaacatcgatcctaatagtagatccgcaaatctgatacattatacaaagctctctagtgagaaaacatcctaatcgtaattataaaagaaaaatacaagaatttattgtataaaaagaaattggcattgtcgatttgtcgtatgcaccaactcaaactcaaatatatagaattatattgtattcctaaaacaataagcagggaagtaactgtgcgagtgcaacgagaataataacaaaagacaagaatttattaagcaaatagaaaccttgagctagccgtttatttatttaatttgagtcgtacgtacggcatataatgatggttgaaaggctagtcacaaaagcaattctttattgaaaatccaactgaacttaacgccatgatgcttcttcatcttacgcgctcgctcatcagcgcgctcttgcaacctcctaatcctcggcgccaacccacacacgaaatcctgcgcccgcttcccctcagccatcaatccttcaagcttctccaatctccaccgttcaacgagaaactccaagatgtctgcgtagtcgttggcggtgtacacgcctatgcgctgcgccacagcggagtagtgctcgaatagcctggggtcatccccatcgtacataaggtgcgccggcatcgtgatcttcttctccatcatgttccctatggccaccattgccccggtggggtccacttccagaagcttctccacgattcttgagtacgcgttctcgtgccgcttctcgtccgctgcgatggtcccgcataggcgcgccagcactggatccccgccctccttcgcgagccgagccgtgttcccgtgcgccacaaatgttgctcgctcttggaatgacgtgtacacaaaccccaaatatgggttgttgtctgtcccagggtccatgccagctgaaatgaggtaatgtacggtcttttcgaccttagccatgtcaaccctcccagagagatacaaataagttctgagcagatccccgtgtctgttttcctcggcggtccaggcccgggtccacacggcccacgggctcgggctcgtgccgctgtcatctttcactccatcaaggttgttgatcatggtctggtaagtgggaagcgcgtcctcggtgaccatatcacccaccagcaccacaaagtactcatcaggtaactctttagtgcgttcgcgaagctccttcacctgatggctgaactcttcatgcggaagggaggggtcagggaggaagttttgtggctgccagcattgctccacgggcttcagcagcggtaggacccactccgaggcccatccctccaaggacttgaaaatttctttcttttctggaggcatcaaattaattttcagctttctctcttaatttttctgttttagtggaacatgttaacgttaagcaacacactttgtctttgatctagcttggcttggctataaatgctaatgtaaagccaaccaggagcaggaacaaagggagagaaatttattcgcccctggtttagtgtgctttctcttgttttacaaatatattattttaaatgtgtaatggagataagcttgttaattttaaatattatatatcttcctttatcacatccttttttttattttattttattcattttccaacataaacaaatattataatactttaaaacttcttttaataatttgttataattgttaaataaagttataattgtcaattaaaaattataaataaaaattaaaattactacgttttgattatcacacatatgttctatattctttattactttaatagataaagagtaaaaaaattgttaattaaaaattaataaatagaattataccaaaaatacgtaagtatatataataaattataaaaatattaaatttttaataaaaatacgtaagtacataatatataataaaaaattaataactatttttaattaattataaaaagatacactctttaccacaccttctcttgagtccttctaaggccttaggactccctagtgggtggtattctctaacatgatgaacactttatgttttaatgttatgcacctgtactagtattagttctctagaactttaaagtgcagtcctggcctgcgaatccatcacttttagggataaatagtgatagccttttcatcattaaaaaacgaaaaaagtataatgagttggaaaaacaaatatttattaaaatagatgatttttgtaacaggatatatcattttaatataatccatcaagccaacacaaaatgaatattagaccaggtgcctcttatatttctacagtaacaaaaataatttattttaataaatgttttcttgatcaacatattataataattagttttcgtttttaacttgtttttggtgaaaaagccaaatattggaccaggtgcagaaatagagataagactatggagccctaaccgcgcgcgagtgagatttcttcccttatgagcagatttttgataaatgctgaacgacctttgccataatgaacaacatggtccccacaatgctcagttttataacctcttggatgaaatcagaaaggtaatgatcttctgctccctgacaatatatttgttaatgctgtctaatgtctacacttaactataaatgcattcttctagttcattagacatttactatattacatgtgcttcgcaatggtatctgttcaccttgtgcaagaggttttcaattgaattgagatattgaaaattgtccaattaatttatggaattatggtaatatgctatattgtttgtgttgacctgctatctatgtatatatatgcacattccatccttattggtattagttcagaggacaaagaaattagtagcactgggaaaggatttgagcttagtcatcatacttggccagactgcattggctgctgctgaaagcagagcacatcatagagttaattagagcacaccagtatttagctcaggtggccacaataatattaagtctgctctgagtccaatacttctgaaaggagatgacatgtggtgtggatcaaacgtcaaagtctctagggccggggaatctgcaacagccaaggaaacatttgagttccgtgggccttacatcattttagcacaatacttgcatctatagtatgaaatttttgtgacaaattagaaatgtaatttaggaaatgttcttaccactgaagtgctcttcaaatcgatctccaccaataatctaatcttctacttggtattctaagctttttcgggtggtgggagtggtacatgtaaaggtactctgacgcttaagtcagtcaataatagaattataataaagttagaatgcgtcaacatttatataatatatagaaaaaaatttgaaattaatcacaaaaactaaaattaagaatttgtctaaaataagaataaagtatctcaattaaaaaataaaaactaaaatcacaaattttaaaaaagtgaaggataaaatgtatcatttaaaaaatgggaaaacgaaaatcacatatttaaaaaaataagagatagaaattgcattttaatatttttttttatttctcttccttttttaattatacttttaatcacattaatgattttattttctatttctcttctttccacctacatacatcccaaagatggagggtgcaattgtaagtttattagcactcttgtttttacctgcatttgtgtgtgctaaccaaattgcattcttctctttacataatgtatttgatttgaattttcataccacatgcaagcatgattacgtacgtgtccatgatcaaatacaaatgctgtctggtactggcaatttggtaaacagccatccatttttttttgtctctaattattctctagaatatctgaagattcctctgtcatcgaattccttgcttggtaacaacgtcgtcaagttattattttgttctttttttttttatcatatttcttattttgttccaagtatgtcatattttgatccatcttgacaagtagattgtcatgtaggaataggaatatcactttaaattttaaagcattgattagtctgtaggcaatattgtcttcttcttcctccttattaatattttttattctgccttcaatcaccagttatgggagatggatgtaatactaaataccatagttgttctgcttgaagtttagttgtatagttgttctgcttgaagtttagttgtgtgtaatgtttcagcgttggcttcccctgtaactgctacaatggtactgaatatatattttttgcattgttcatttttttcttttacttaatcttcattgctttgaaattaataaaacaaaaagaaggaccgaatagtttgaagtttgaactattgcctattcatgtaacttattcacccaatcttatatagtttttctggtagagatcattttaaattgaaggatataaattaagaggaaatacttgtatgtgatgtgtggcaatttggaagatcatgcgtagagagtttaatggcaggttttgcaaattgacctgtagtcataattacactgggccctctcggagttttgtgcctttttgttgtcgctgtgtttggttctgcatgttagcctcacacagatatttagtagttgttgttctgcatataagcctcacacgtatactaaacgagtgaacctcaaaatcatggccttacacctattgagtgaaattaatgaacagtgcatgtgagtatgtgactgtgacacaacccccggttttcatattgcaatgtgctactgtggtgattaaccttgctacactgtcgtccttgtttgtttccttatgtatattgataccataaattattactagtatatcattttatattgtccataccattacgtgtttatagtctctttatgacatgtaattgaattttttaattataaaaaataataaaacttaattacgtactataaagagatgctcttgactagaattgtgatctcctagtttcctaaccatatactaatatttgcttgtattgatagcccctccgttcccaagagtataaaactgcatcgaataatacaagccactaggcatggtaaattaaattgtgcctgcacctcgggatatttcatgtggggttcatcatatttgttgaggaaaagaaactcccgaaattgaattatgcatttatatatcctttttcatttctagatttcctgaaggcttaggtgtaggcacctagctagtagctacaatatcagcacttctctctattgataaacaattggctgtaatgccgcagtagaggacgatcacaacatttcgtgctggttactttttgttttatggtggcaacagctgctacttcatcatttttccctgttacttcaccctcgccggactctggtggagcaggcagcaaacttggtggtgggcctgcaaaccttggaggactaaaatccaaatctgcgtcttctggtggcttgaaggcaaaggcgcaagccccttcgaaaattaatggaaccacagttgttacatctaaagaaagcttcaagcatgatgatgatctaccttcgcctccccccagaacttttatcaaccagttgcctgattggagcatgcttcttgctgctatcacaacaattttcttggccgctgaaaagcagtggatgatgcttgattggaagccacggcgacctgacatgcttattgacccctttgggataggaaaaattgttcaggatggtcttgtgttccgtgaaaacttttctattagatcatatgagattggtgctgatcgtaccgcatctatagaaacagtaatgaaccatttgcaagaaactgcacttaatcatgttaaaagtgctgggcttcttggtgatggctttggttccacgccagaaatgtgcaaaaagaacttgatatgggtggttactcggatgcaggttgtggtggaacgctatcctacatggggtgacatagttcaagtggacacttgggtttctggatcagggaagaatggtatgcgccgtgattggcttttacgtgactgcaaaactggtgaaatcttgacaagagcttccagtgtttgggtcatgatgaataagctaacacggaggctgtctaaaattccagaagaagtcagacaggagataggatcttattttgtggattctgatccaattctggaagaggataacagaaaactgactaaacttgacgacaacacagcggattatattcgtaccggtttaagtcctaggtggagtgatctagatatcaatcagcatgtcaacaatgtgaagtacattggctggattctggagagtgctccacagccaatcttggagagtcatgagctttcttccatgactttagagtataggagagagtgtggtagggacagtgtgctggattccctgactgctgtatctggggccgacatgggcaatctagctcacagcgggcatgttgagtgcaagcatttgcttcgactggaaaatggtgctgagattgtgaggggcaggactgagtggaggcccaaacctgtgaacaactttggtgttgtgaaccaggttccagcagaaagcacctaatggagcaaccaatgggccatagtgggagttatggaagttttgtcatgtattagtacataattagtagaatgttataaataagtggatttgccgcgtaatgactttgtgtgtattgtgaaacagcttgttgcgatcatggttataatgtaaaaataattctggtattaattacatgtggaaagtgttctgcttatagctttctgcctaaaatgcacgctgcacgggacaatatcattggtaatttttttaaaatctgaattgaggctactcataatactatccataggacatcaaagacatgttgcattgactttaagcagaggttcatctagaggattactgcataggcttgaactacaagtaatttaagggacgagagcaactttagctctaccacgtcgttttacaaggttattaaaatcaaattgatcttattaaaactgaaaatttgtaataaaatgctattgaaaaattaaaatatagcaaacacctaaattggactgatttttagattcaaatttaataattaatctaaattaaacttaaattttataatatatgtcttgtaatatatcaagtttttttttttattattgagtttggaaacatataataaggaacattagttaatattgataatccactaagatcgacttagtattacagtatttggatgatttgtatgagatattcaaacttcactcttatcataatagagacaaaagttaatactgatggtggagaaaaaaaaatgttattgggagcatatggtaagataagacggataaaaatatgctgcagcctggagagctaatgtattttttggtgaagttttcaagtgacaactattcatgatgagaacacaataatattttctacttacctatcccacataaaatactgattttaataatgatgataaataatgattaaaa